(19) United States
(12) Patent Application Publication
(10) Pub. No.: US 2014/0196174 A1
(43) Pub. Date: Jul. 10, 2014

(54) Development of Phytophthora Resistant Potato with Increased Yield

(71) Applicants: Holger Schultheiss, Bohl-Iggelheim (DE); Timo Böhme, Ludwigshafen (DE)

(72) Inventors: Holger Schultheiss, Bohl-Iggelheim (DE); Timo Böhme, Ludwigshafen (DE)

(73) Assignee: BASF PLANT SCIENCE COMPANY GMBH, Ludwigshafen (DE)

(21) Appl. No.: 14/115,711

(22) PCT Filed: May 24, 2012
(86) PCT No.: PCT/IB12/52591

§ 371 Date: Mar. 17, 2014

Related U.S. Application Data
(60) Provisional application No. 61/489,271, filed on May 24, 2011.

(30) Foreign Application Priority Data
May 24, 2011 (EP) 11167251.5

(51) Int. Cl. C12N 15/82; C12Q 1/68

(52) U.S. Cl. CPC C12N 15/8279; C12Q 1/6895
USPC 800/279; 800/301; 435/417; 435/6.12; 536/23.6

(57) ABSTRACT

The present invention relates to transgenic potato plants having an increased resistance against Phytophthora infestans and a comparable yield of potato tubers compared with the wildtype potato plants, wherein the blb1-gen and blb2-gen are integrated within a specific genetic background into the potato plant.

The present invention relates to transgenic potato plants having an increased resistance against Phytophthora infestans and a comparable yield of potato tubers compared with the wildtype potato plants, wherein the blb1-gene and blb2-gene are integrated within a specific genetic background into the potato plant.

Late blight caused by the oomycete Phytophthora infestans is one of the most severe threats to potato production worldwide. Despite many years of resistance breeding, the only effective way to prevent crop failures or reduced yields is the application of fungicides that prevent or cure an infection by P. infestans. As the disease development of late blight is extremely fast, it is necessary to run a tight fungicide regime, which has to start before first symptoms occur. Furthermore, P. infestans seems to have a high potential to adapt to specific fungicides and to develop resistance, as already seen in the case of metalaxyl-fungicides (Gisi U, Cohen Y (1996) Resistance to phenylamide fungicides: A case study with Phytophthora infestans involving mating type and race structure Annual Rev Phytopathol. 34: 549-572).

In several Western European countries, legislation on the use of plant protection products is becoming more restrictive regarding the application of specific fungicides, making chemical control of the disease and the prevention of resistance development more difficult.

An alternative and/or complementary approach to the use of fungicides is the development of potato cultivars that harbour improved resistance to P. infestans.

In recent years, two potato varieties containing S. bulbocastanum derived resistance were developed via conventional breeding. Both varieties, Toluca and Bionica, contain a single S. bulbocastanum resistance gene that confers full resistance against P. infestans. But from an agronomical point of view the two potato varieties do not match modern potato varieties in terms of yield potential.

As the introgression of the S. bulbocastanum derived resistance into modern potato varieties turned out to be difficult and time consuming, a much more efficient approach is the isolation of the genes that code for Phytophthora resistance in S. bulbocastanum and their transfer into current potato cultivars by biotechnological methods.

To generate the durably resistant potato plants, the Rpi-blb1 and the Rpi-blb2 genes were combined under control of their native regulation elements. The resultant vector construct contained the genomic sequence of the Rpi-blb1 gene under control of the native Rpi-blb1 promoter and Rpi-blb1 terminator, all derived from S. bulbocastanum, in combination with the genomic sequence of the Rpi-blb2 gene under control of the native Rpi-blb2 promoter and Rpi-blb2 terminator all from S. bulbocastanum (WO 2008/034876). The obtained transgenic potatoes expressing blb1-protein and blb2-protein showed increased resistance to Phytophthora infestans. However, it was found that the yield of the developed potato plants was decreased.

It is an object of the present invention to provide potato plants having an improved resistance to Phytophthora infestans and a comparable yield with the wildtype potato.

The present invention may be understood more readily by reference to the following detailed description of the preferred embodiments of the invention and the examples included herein. Unless otherwise noted, the terms used herein are to be understood according to conventional usage by those of ordinary skill in the relevant art. In addition to the definitions of terms provided herein, definitions of common terms in molecular biology may also be found in Rieger et al., 1991 Glossary of genetics: classical and molecular, 5th Ed., Berlin: Springer-Verlag; and in Current Protocols in Molecular Biology, F. M. Ausubel et al., Eds., Current Protocols, a joint venture between Greene Publishing Associates, Inc. and John Wiley & Sons, Inc., (1998 Supplement). It is to be understood that as used in the specification and in the claims, “a” or “an” can mean one or more, depending upon the context in which it is used. Thus, for example, reference to “a cell” can mean that at least one cell can be utilized. It is to be understood that the terminology used herein is for the purpose of describing specific embodiments only and is not intended to be limiting.

Throughout this application, various publications are referenced. The disclosures of all of these publications and those references cited within those publications in their entireties are hereby incorporated by reference into this application in order to more fully describe the state of the art to which this invention pertains. Standard techniques for cloning, DNA isolation, amplification and purification, for enzymatic reactions involving DNA ligase, DNA polymerase, restriction endonucleases and the like, and various separation techniques are those known and commonly employed by those skilled in the art. A number of standard techniques are described in Sambrook et al., 1989 Molecular Cloning, Second Edition, Cold Spring Harbor Laboratory, Plainview, N.Y.; Maniatis et al., 1982 Molecular Cloning, Cold Spring Harbor Laboratory, Plainview, N.Y.; Wu (Ed.) 1993 Meth. Enzymol. 218, Part I; Wu (Ed.) 1979 Meth Enzymol. 68; Wu et al., (Eds.) 1983 Meth. Enzymol. 100 and 101; Grossman and Moldave (Eds.) 1980 Meth. Enzymol. 65; Miller (Ed.) 1972 Experiments in Molecular Genetics, Cold Spring Harbor Laboratory, Cold Spring Harbor, N.Y.; Old and Primrose, 1981 Principles of Gene Manipulation, University of California Press, Berkeley; Schleif and Wensink, 1982 Practical Methods in Molecular Biology; Glover (Ed.) 1985 DNA Cloning Vol. I and II, IRL Press, Oxford, UK; Hames and Higgins (Eds.) 1985 Nucleic Acid Hybridization, IRL Press, Oxford, UK; and Setlow and Hollaender 1979 Genetic Engineering: Principles and Methods, Vols. 1-4, Plenum Press, New York. Abbreviations and nomenclature, where employed, are deemed standard in the field and commonly used in professional journals such as those cited herein.

The object of the present invention is solved by the provision of Phytophthora-resistant-transgenic potato plant, seed, tuber, plant cell or tissue thereof having a specific integration site for the blb1-gene and blb2-gene.

One embodiment according to the present invention provides a Phytophthora-resistant transgenic potato plant, seed, tuber, plant cell or tissue, preferably comprising a nucleotide sequence having at least 80% identity with SEQ ID NO: 1. (cf. FIG. 2a).

One embodiment according to the present invention provides a Phytophthora-resistant transgenic potato plant, seed, tuber, plant cell or tissue comprising a nucleotide sequence having at least 80% identity with SEQ ID NO: 2 or SEQ ID NO: 3 flanked by flanking regions having at least 80% identity with SEQ ID NO: 20 and/or SEQ ID NO: 21 (cf. FIGS. 2b,c,e).

SEQ ID NO: 1 refers to the part of the recombinant construct inserted into the plant genome including the blb1-gene, blb2-gene and ahas-gene, wherein SEQ ID NO: 1 further includes the flanking genomic sequences of the plant (cf. FIG. 2a).

SEQ ID NO: 2 refers to the part of the recombinant construct inserted into the plant genome including the blb1-gene, blb2-gene and the ahas-gene. Preferably, the blb1-gene, blb2-gene and optionally the ahas-gene are expressed, if a sequence having at least 80% identity to SEQ ID NO: 2 is inserted into the plant genome. Preferably, the blb1-gene, blb2-gene and/or ahasgene are expressed, if a sequence having at least 80% identity to SEQ ID NO: 2 is inserted into the plant genome (cf. FIG. 2b).

SEQ ID NO: 3 refers to the part of the recombinant construct inserted into the plant genome including the blb1-gene, the blb2-gene but without the ahas-marker-gene. Preferably, the blb1-gene, and blb2-gene are expressed, if a sequence having at least 80% identity to SEQ ID NO: 3 is inserted into the plant genome (cf. FIG. 2c).

SEQ ID NOs: 2 and/or 3 may be referred to herein later as insert.

SEQ ID NO: 20 refers to the left flanking region of the insert having SEQ ID NOs: 2 or 3.

SEQ ID NO: 21 refers to the right flanking region of the insert having SEQ ID NOs: 2 or 3.

The term “flanking region” refers the region of the plant genome flanking either the right or left site of the insert which is integrated into the plant genome.

One embodiment according to the present invention provides a Phytophthora-resistant transgenic potato plant, seed, tuber, plant cell or tissue thereof comprising
a) a recombinant construct having at least 80% identity with SEQ ID NO: 2 or SEQ ID NO:3 and
   further comprising a junction sequence selected from the group consisting of
b)
   i) a nucleic acid sequence that can be used to amplify a nucleotide fragment of between 126 and 136 basepairs using a polymerase chain reaction with two primers having the nucleotide sequences of SEQ ID NO: 4 and SEQ ID NO: 5,
   ii) a nucleic acid sequence that can be used to amplify a nucleotide fragment of between 505 and 515 basepairs using a polymerase chain reaction with two primers having the nucleotide sequences of SEQ ID NO: 6 and SEQ ID NO: 7,
   iii) a nucleic acid sequence that can be used to amplify a nucleotide fragment of between 625 and 635 basepairs using a polymerase chain reaction with two primers having the nucleotide sequences of SEQ ID NO: 8 and SEQ ID NO: 9, and/or
   iv) a nucleic acid sequence that can be used to amplify a nucleotide fragment of between 4752 and 4762 basepairs using a polymerase chain reaction with two primers having the nucleotide sequences of SEQ ID NO: 10 and SEQ ID NO: 11,
and/or
   further comprising a junction sequence selected from the group consisting of
   v) a nucleic acid sequence that can be used to amplify a nucleotide fragment of between 282 and 292 basepairs using a polymerase chain reaction with two primers having the nucleotide sequences of SEQ ID NO: 12 and SEQ ID NO: 13,
   vi) a nucleic acid sequence that can be used to amplify a nucleotide fragment of between 877 and 887 basepairs using a polymerase chain reaction with two primers having the nucleotide sequences of SEQ ID NO: 14 and SEQ ID NO: 15,
   vii) a nucleic acid sequence that can be used to amplify a nucleotide fragment of between 827 and 837 basepairs using a polymerase chain reaction with two primers having the nucleotide sequences of SEQ ID NO: 16 and SEQ ID NO: 17, and/or
   viii) a nucleic acid sequence that can be used to amplify a nucleotide fragment of between 9905 and 9915 basepairs using a polymerase chain reaction with two primers having the nucleotide sequences of SEQ ID NO: 18 and SEQ ID NO: 19.

A preferred embodiment of the present invention provides a Phytophthora-resistant transgenic potato, seed, tuber, plant cell or tissue thereof
a) a recombinant construct having at least 80% identity with SEQ ID NO: 2 or SEQ ID NO: 3 and
   further comprising a junction sequence selected from the group consisting of
b)
   i) a nucleic acid sequence that can be used to amplify a nucleotide fragment of 131 basepairs using a polymerase chain reaction with two primers having the nucleotide sequences of SEQ ID NO: 4 and SEQ ID NO: 5,
   ii) a nucleic acid sequence that can be used to amplify a nucleotide fragment 510 basepairs using a polymerase chain reaction with two primers having the nucleotide sequences of SEQ ID NO: 6 and SEQ ID NO: 7,
   iii) a nucleic acid sequence that can be used to amplify a nucleotide fragment of 630 basepairs using a polymerase chain reaction with two primers having the nucleotide sequences of SEQ ID NO: 8 and SEQ ID NO: 9, and/or
   iv) a nucleic acid sequence that can be used to amplify a nucleotide fragment of 4757 basepairs using a polymerase chain reaction with two primers having the nucleotide sequences of SEQ ID NO: 10 and SEQ ID NO: 11
and/or
   further comprising a junction sequence selected from the group consisting of
   v) a nucleic acid sequence that can be used to amplify a nucleotide fragment of 287 basepairs using a polymerase chain reaction with two primers having the nucleotide sequences of SEQ ID NO: 12 and SEQ ID NO: 13,
   vi) a nucleic acid sequence that can be used to amplify a nucleotide fragment of 882 basepairs using a polymerase chain reaction with two primers having the nucleotide sequence of SEQ ID NO: 14 and SEQ ID NO: 15,
   vii) a nucleic acid sequence that can be used to amplify a nucleotide fragment of 832 basepairs using a polymerase chain reaction with two primers having the nucleotide sequences of SEQ ID NO: 16 and SEQ ID NO: 17, and/or
   viii) a nucleic acid sequence that can be used to amplify a nucleotide fragment of 9910 basepairs using a polymerase chain reaction with two primers having the nucleotide sequences of SEQ ID NO: 18 and SEQ ID NO: 19.

A nucleic acid sequence that can be used to amplify a nucleotide fragment of a certain length using a polymerase chain reaction with two primers means the product of said polymerase chain reaction with two primers. In particular, this means a nucleic acid sequence that is amplified to a nucleotide fragment of a certain length using a polymerase chain reaction with two primers.

A junction sequence includes either a right or left part of the recombinant construct inserted into the plant genome and partially includes plant genomic sequences of the flanking region of said right or left part of the recombinant construct. In particular, the junction sequence comprises either a left part of the recombinant construct having at least 80% identity with SEQ ID NO: 2 or 3 and a part of the flanking region having at least 80% identity to SEQ ID NO: 20 or a right part of the recombinant construct having at least 80% identity to SEQ ID NO: 2 or 3 and a part of the flanking region having at least 80% identity with SEQ ID NO: 21. Identity with respect to partial sequences of the recombinant construct means in this case identity over the entire length of said left or right part of the recombinant construct (FIG. 2e).

In particular, there is at least a partial overlap of either a part of the left part of the recombinant construct having at least 80% identity with SEQ ID NO: 2 or 3 and the junction sequence having at least 80% identity with SEQ ID NO: 22 or a part of the right part of the recombinant construct having at least 80% identity with SEQ ID NO: 2 or 3 and the junction sequence having at least 80% identity with SEQ ID NO: 23. Identity with respect to partial sequences of the recombinant construct means in this case identity over the entire length of said left or right part of the recombinant construct (FIG. 2e).

PCR means polymerase chain reaction, i.e. the selective enrichment of nucleic acids of defined length within a mixture of nucleic acids with primers specific for said nucleic acid by using Taq-polymerase or the like (U.S. Pat. No. 5,656,493; Sambrook et al. 1989, Molecular Cloning, Second Edition, Cold Spring Harbor Laboratory, Plainview, N.Y.).

An alternative embodiment provides a Phytophthora-resistant transgenic potato, seed, tuber, plant cell or tissue comprising
a) a recombinant construct having at least 80% identity with SEQ ID NO: 2 or SEQ ID NO: 3 and
   further comprising a junction sequence selected from the group consisting of
   a sequence having at least 80% identity to SEQ ID NO: 22 and/or SEQ ID NO: 23.

A transgenic potato plant, seed, tuber, plant cell or tissue according to the present invention comprising the recombinant construct which is amplifiable as defined above may be obtained by propagation or crossing a potato plant with a potato plant obtained from the seeds of the above described plants of elite-event D and subsequent selection of the plants carrying the recombinant construct by detection with PCR using the above defined primer pairs. The berries containing the may be hand-harvested, extracted and dried recently. The seeds may be stored at room temperature. Seed may be treated with 0.04% GA (giberellic acid) in order to break dormacy and enhance germination.

In one embodiment for crossing the pollen of the father plant is transferred from its stamen to the isolated carpel of the mother plant. The true seed bearing berries are harvested and the seeds are separated from the peel and flesh of the berries. The seeds are replanted, grown to plants and subsequently the plants carrying the recombinant construct are selected by detection with PCR using the above defined primer pairs.

The mother plant and/or the father plants may be the phythophthora-resistant transgenic potato plant according to the present invention. In one embodiment the mother plant is phythophthora-resistant transgenic potato plant according to the present invention and the father plant may be a non-transgenic plant, e.g. selected from the group consisting of Agria, Sarpo Mira, Cara, Valor, Innovator, Diamant and Bintje. In an alternative embodiment the father plant is the phythophthora-resistant transgenic potato plant according to the present invention and the mother plant may be a non-transgenic plant, e.g. selected from the group consisting of Agria, Sarpo Mira, Cara, Valor, Innovator, Diamant and Bintje.

One embodiment of the present invention provides a method for providing a Phytophthora-resistant transgenic potato plant or part thereof comprising the following steps:
a) introducing a recombinant nucleic acid having at least 80% identity with SEQ-ID- No. 2 or SEQ-ID-No. 3 into the genome of potato plant cells,
b) integrating said recombinant nucleic acid into the genome,
c) regenerating plant from said plant cells,
d) selecting plant comprising a nucleic acid having at least 80% identity with SEQ-ID-No. 2 or SEQ-ID-No. 3 and a junction sequence selected from the group consisting of a sequence having at least 80% identity to SEQ-ID-No. 22 and/or SEQ-ID-No. 23.

One embodiment of the present invention provides a method for providing a Phytophthora-resistant transgenic potato plant or part thereof comprising the following steps:
a) introducing a recombinant nucleic acid having at least 80% identity with SEQ ID NO: 2 or SEQ ID NO: 3 into the genome of potato plant cells,
b) integrating said recombinant nucleic acid into the genome,
c) regenerating plant from said plant cells,
d) selecting plant comprising a nucleic acid having at least 80% identity with SEQ ID NO: 2 or SEQ ID NO: 3 and a junction sequence selected from the group consisting of
   i) a nucleic acid sequence that can be used to amplify a nucleotide fragment of between 126 and 136 basepairs using a polymerase chain reaction with two primers having the nucleotide sequences of SEQ ID NO: 4 and SEQ ID NO: 5,
   ii) a nucleic acid sequence that can be used to amplify a nucleotide fragment of between 505 and 515 basepairs using a polymerase chain reaction with two primers having the nucleotide sequences of SEQ ID NO: 6 and SEQ ID NO: 7,
   iii) a nucleic acid sequence that can be used to amplify a nucleotide fragment of between 625 and 635 basepairs using a polymerase chain reaction with two primers having the nucleotide sequences of SEQ ID NO: 8 and SEQ ID NO: 9, and/or
   iv) a nucleic acid sequence that can be used to amplify a nucleotide fragment of between 4752 and 4762 basepairs using a polymerase chain reaction with two primers having the nucleotide sequences of SEQ ID NO: 10 and SEQ ID NO: 11,
   and/or
   further comprising a junction sequence selected from the group consisting of
   v) a nucleic acid sequence that can be used to amplify a nucleotide fragment of between 282 and 292 basepairs, using a polymerase chain reaction with two primers having the nucleotide sequences of SEQ ID NO: 12 and SEQ ID NO: 13,
   vi) a nucleic acid sequence that can be used to amplify a nucleotide fragment of between 877 and 887 basepairs, using a polymerase chain reaction with two primers having the nucleotide sequences of SEQ ID NO: 14 and SEQ ID NO: 15,
   vii) a nucleic acid sequence that can be used to amplify a nucleotide fragment of between 827 and 837 basepairs, using a polymerase chain reaction with two primers having the nucleotide sequences of SEQ ID NO: 16 and SEQ ID NO: 17 and/or
   viii) a nucleic acid sequence that can be used to amplify a nucleotide fragment of between 9905 and 9915 basepairs, using a polymerase chain reaction with two primers having the nucleotide sequence of SEQ ID NO: 18 and SEQ ID NO: 19.

One method of the present invention provides a method for providing a Phytophthora-resistant transgenic potato plant or part thereof,

wherein in step d) plants are selected comprising a nucleic acid having at least 80% identity with SEQ ID NO: 2 or SEQ ID NO: 3 and a junction sequence selected from the group consisting of
i) a nucleic acid sequence that can be used to amplify a nucleotide fragment of 131 basepairs using a polymerase chain reaction with two primers having the nucleotide sequences of SEQ ID NO: 4 and SEQ ID NO: 5,
ii) a nucleic acid sequence that can be used to amplify a nucleotide fragment 510 basepairs using a polymerase chain reaction with two primers having the nucleotide sequences of SEQ ID NO: 6 and SEQ ID NO: 7,
iii) a nucleic acid sequence that can be used to amplify a nucleotide fragment of 630 basepairs using a polymerase chain reaction with two primers having the nucleotide sequences of SEQ ID NO: 8 and SEQ ID NO: 9, and/or
iv) a nucleic acid sequence that can be used to amplify a nucleotide fragment of 4757 basepairs using a polymerase chain reaction with two primers having the nucleotide sequences of SEQ ID NO: 10 and SEQ ID NO: 11,
and/or
v) a nucleic acid sequence that can be used to amplify a nucleotide fragment of 287 basepairs using a polymerase chain reaction with two primers having the nucleotide sequences of SEQ ID NO: 12 and SEQ ID NO: 13,
vi) a nucleic acid sequence that can be used to amplify a nucleotide fragment of 882 basepairs using a polymerase chain reaction with two primers having the nucleotide sequences of SEQ ID NO: 14 and SEQ ID NO: 15,
vii) a nucleic acid sequence that can be used to amplify a nucleotide fragment of 832 basepairs using a polymerase chain reaction with two primers having the nucleotide sequences of SEQ ID NO: 16 and SEQ ID NO: 17, and/or
vii) a nucleic acid sequence that can be used to amplify a nucleotide fragment of 9910 basepairs using a polymerase chain reaction with two primers having the nucleotide sequences of SEQ ID NO: 18 and SEQ ID NO: 19.

One embodiment of the present invention provides a kit for the detection of the specific integration place, in particular for the detection of elite-event D comprising the primer pairs
SEQ ID NO: 4 and 5,
SEQ ID NO: 6 and 7,
SEQ ID NO: 8 and 9,
SEQ ID NO: 10 and 11,
SEQ ID NO: 12 and 13,
SEQ ID NO: 14 and 15,
SEQ ID NO: 16 and 17, and/or
SEQ ID NO: 18 and 19.

The kit disclosed can be used for purposes of quality control (e.g., purity of seed lots), detection of the specific integration place in plant material or material comprising or derived from plant material, such as french fries, potato meal, mash potatoes etc. but not limited to food or feed products.

Briefly, genomic DNA is amplified by PCR using a primer which specifically recognizes the 5′ or 3′ flanking sequence of the insertion site in the elite-event D, in particular the above mentioned primer pairs, e.g. primers SEQ ID NO: 4 and SEQ ID NO: 5, SEQ ID NO: 6 and SEQ ID NO: 7, SEQ ID NO: 8 and SEQ ID NO: 9, SEQ ID NO: 10 and SEQ ID NO: 11, SEQ ID NO: 12 and SEQ ID NO: 13, SEQ ID NO: 14 and SEQ ID NO: 15, SEQ ID NO: 16 and SEQ ID NO: 17, SEQ ID NO: 18 and SEQ ID NO: 19, respectively. If PCR using above mentioned primer combinations on the plant material yields a fragment of
126 to 136 by (131 bp),
505 to 515 by (510 bp),
625 to 536 by (630 bp),
282 to 292 by (287 bp),
877 to 887 by (882 bp),
827 to 837 by (832 bp),
4752 to 4762 by (4757 bp)
9905 to 9915 by (9910), respectively,
   the transgenic plant is determined to have the herein defined specific integration place, e.g. the selected elite-event D. The fragment length can be determined by gel electrophoresis using markers Sambrook et al., 1989 Molecular Cloning, Second Edition, Cold Spring Harbor Laboratory, plainview, N.Y.).

One embodiment of the present invention provides a detection method for a specific integration place, preferably for the identification of elite-event D, comprising the steps of
a) isolating DNA from a potato plant as a test sample,
b) exposing the test sample, a positive and a negative sample, a primer pair as defined above under PCR-conditions, and
c) evaluating the amplification of a DNA-fragment
   i) of between 126 and 136 basepairs when using a polymerase chain reaction with two primers having the nucleotide sequences of SEQ ID NO: 4 and SEQ ID NO: 5,
   ii) of between 505 and 515 basepairs when using a polymerase chain reaction with two primers having the nucleotide sequences of SEQ ID NO: 6 and SEQ ID NO: 7,
   iii) of 625 and 635 basepairs when using a polymerase chain reaction with two primers having the nucleotide sequences of SEQ ID NO: 8 and SEQ ID NO: 9, and/or
   iv) of 4752 and 4762 basepairs when using a polymerase chain reaction with two primers having the nucleotide sequences of SEQ ID NO: 10 and SEQ ID NO: 11,
and/or
   v) of between 282 and 292 basepairs when using a polymerase chain reaction with two primers having the nucleotide sequence of SEQ ID NO: 12 and SEQ ID NO: 13,
   vi) of 877 and 292 basepairs when using a polymerase chain reaction with two primers having the nucleotide sequences of SEQ ID NO: 14 and SEQ ID NO: 15,
   vii) of 827 and 837 basepairs when using a polymerase chain reaction with two primers having the nucleotide sequences of SEQ ID NO: 16 and SEQ ID NO: 17, and/or
   viii)a nucleic acid sequence that can be used to amplify a nucleotide fragment of 9905 and 9915 basepairs, using a polymerase chain reaction with two primers having the nucleotide sequences of SEQ ID NO: 18 and SEQ ID NO: 19
   compared with said positive and negative control.

In a preferred embodiment in step e)

evaluating means the amplification of a nucleotide fragment selected from the group consisting of a nucleotide fragment
i) of 131 basepairs when using a polymerase chain reaction with two primers having the nucleotide sequences of SEQ ID NO: 4 and SEQ ID NO: 5,
ii) of 510 basepairs when using a polymerase chain reaction with two primers having the nucleotide sequences of SEQ ID NO: 6 and SEQ ID NO: 7,
iii) of 630 basepairs when using a polymerase chain reaction with two primers having the nucleotide sequences of SEQ ID NO: 8 and SEQ ID NO: 9, and/or
iv) of 4757 basepairs when using a polymerase chain reaction with two primers having the nucleotide sequences of SEQ ID NO: 10 and SEQ ID NO: 11

and/or
of a nucleotide fragment selected from the group consisting of a nucleotide fragment
v) of 287 basepairs when using a polymerase chain reaction with two primers having the nucleotide sequences of SEQ ID NO: 12 and SEQ ID NO: 13,
vi) of 882 basepairs when using a polymerase chain reaction with two primers having the nucleotide sequences of SEQ ID NO: 14 and SEQ ID NO: 15,
vii) of 832 basepairs when using a polymerase chain reaction with two primers having the nucleotide sequences of SEQ ID NO: 16 and SEQ ID NO: 17, and/or
viii) a nucleic acid sequence that can be used to amplify a nucleotide fragment of 9910 basepairs, using a polymerase chain reaction with two primers having the nucleotide sequences of SEQ ID NO: 18 and SEQ ID NO: 19 compared with said positive and negative control.

The test sample comprises genomic DNA isolated from transformed plant material, e.g. from plants, seed, tuber, plant cells or an tissue thereof. The positive sample is a sample comprising genomic DNA isolated from plants including SEQ ID NO: 1. The negative sample is a sample comprising genomic DNA isolated from the non-transgenic original variety used for transformation, e.g. the Fontane variety.

The PCR reaction can be run with various DNA polymerases, such as the Pfu Ultra, Pfu Turbo or Herculase DNA Polymerase (Agilent Technologies, Santa Clara, Calif., US). The composition for the protocol of the Pfu Ultra, Pfu Turbo or Herculase DNA polymerase may be as follows: 1× PCR buffer, 0.2 mM of each dNTP, 1 μg genomic DNA of Sample, 50 pmol forward primer, 50 pmol reverse primer, 1 u Pfu Ultra, Pfu Turbo or Herculase DNA polymerase.

The amplification cycles may be as follows:

1 cycle of 60 seconds at 98° C., followed by 35 cycles of in each case 10 seconds at 98° C., 30 seconds at the annealing temperature given in the table below and 60 seconds per 1000 bp product length (see table below) at 72° C., followed by 1 cycle of 10 minutes at 72° C., then 4° C.

TABLE 1

| | | | | | | Annealig |
|---|---|---|---|---|---|---|
| | | | SEQ | | SEQ | temper- |
| | | Sense | ID | Antisense | ID | ature |
| Event D | Border | primer | NO: | primer | NO: | [° C.] |
| | LB | TCAAAC | 4 | CCAGTTCCC | 5 | 52 |
| | | GGATGT | | AATTGACTA | | |
| | | TAATTC | | CTAGAAA | | |
| | | AGTACA | | | | |
| | | TT | | | | |
| | LB | TCTGTT | 6 | CTCAGAAGA | 7 | 48 |
| | | GAATTA | | AAGAATTGT | | |
| | | CGTTAA | | TC | | |
| | | GC | | | | |
| | LB | GTTTCT | 8 | GCCCATTCT | 9 | 51 |
| | | TAAGAT | | CTATTTTAC | | |
| | | TGAATC | | TCACTAA | | |
| | | CTGTTGC | | | | |
| | RB | CCAAGA | 12 | AAATTCATG | 13 | 52 |
| | | TAGTGT | | GTAGGAACT | | |
| | | TTCAGG | | GGAGAG | | |
| | | AAAGTT | | | | |
| | | ATT | | | | |
| | RB | AACTGA | 14 | GAGTCAGTT | 15 | 49 |
| | | ATTTTG | | AAATTAACT | | |
| | | GGATTG | | GCTTCAG | | |
| | | AG | | | | |
| | RB | ACAAGA | 16 | GAAGTTCGA | 17 | 49 |
| | | ATAGCA | | ACAACATTC | | |
| | | AGGATT | | TT | | |
| | | ATCC | | | | |
| Left | LB | GTGAAC | 10 | CAACTAATA | 11 | 52 |
| flanking | | TAGGAA | | AAACCAAGG | | |
| region | | ACCTAA | | AC | | |
| to blb1 | | ATC | | | | |
| Right | RB | AACTGA | 18 | ATGTAGCAG | 19 | 50 |
| flanking | | ATTTTG | | CATTGAGTT | | |
| region | | GGATTG | | TT | | |
| to blb1 | | AG | | | | |

One embodiment according to the present invention provides a plant, tuber, seed, detectable by the above defined kit or by the above defined detection method.

One embodiment of the present invention provides a polynucleotide comprising
a) a recombinant nucleic acid having at least 80% identity with SEQ ID NO: 2 or SEQ ID NO: 3 and
b) further comprising a junction sequence selected from the group consisting of
   i) a nucleic acid sequence that can be used to amplify a nucleotide fragment of between 126 and 136 basepairs using a polymerase chain reaction with two primers having the nucleotide sequences of SEQ ID NO: 4 and SEQ ID NO: 5,
   ii) a nucleic acid sequence that can be used to amplify a nucleotide fragment of between 505 and 515 basepairs using a polymerase chain reaction with two primers having the nucleotide sequences of SEQ ID NO: 6 and SEQ ID NO: 7,
   iii) a nucleic acid sequence that can be used to amplify a nucleotide fragment of between 625 and 635 basepairs using a polymerase chain reaction with two primers having the nucleotide sequences of SEQ ID NO: 8 and SEQ ID NO: 9, and/or
   iv) a nucleic acid sequence that can be used to amplify a nucleotide fragment of between 4752 and 4762 basepairs using a polymerase chain reaction with two primers having the nucleotide sequences of SEQ ID NO: 10 and SEQ ID NO: 11,
and/or
   further comprising a junction sequence selected from the group consisting of
   v) a nucleic acid sequence that can be used to amplify a nucleotide fragment of between 282 and 292 basepairs, using a polymerase chain reaction with two primers having the nucleotide sequences of SEQ ID NO: 12 and SEQ ID NO: 13
   vi) a nucleic acid sequence that can be used to amplify a nucleotide fragment of between 877 and 887 basepairs, using a polymerase chain reaction with two primers having the nucleotide sequences of SEQ ID NO: 14 and SEQ ID NO: 15
   vii) a nucleic acid sequence that can be used to amplify a nucleotide fragment of between 827 and 837 basepairs, using a polymerase chain reaction with two primers having the nucleotide sequences of SEQ ID NO: 16 and SEQ ID NO: 17 and/or
   viii) a nucleic acid sequence that can be used to amplify a nucleotide fragment of between 9905 and 9915 basepairs, using a polymerase chain reaction with two primers having the nucleotide sequences of SEQ ID NO: 18 and SEQ ID NO: 19.

One preferred embodiment of the present invention provides a polynucleotide comprising
a) a recombinant nucleic acid having at least 80% identity with SEQ ID NO: 2 or SEQ ID NO: 3 and
b) further comprising a junction sequence selected from the group consisting of
   i) a nucleic acid sequence that can be used to amplify a nucleotide fragment of 131 basepairs, using a polymerase chain reaction with two primers having the nucleotide sequences of SEQ ID NO: 4 and SEQ ID NO: 5,
   ii) a nucleic acid sequence that can be used to amplify a nucleotide fragment 510 basepairs, using a polymerase chain reaction with two primers having the nucleotide sequences of SEQ ID NO: 6 and SEQ ID NO: 7,
   iii) a nucleic acid sequence that can be used to amplify a nucleotide fragment of 630 basepairs, using a polymerase chain reaction with two primers having the nucleotide sequences of SEQ ID NO: 8 and SEQ ID NO: 9, and/or
   iv) a nucleic acid sequence that can be used to amplify a nucleotide fragment of 4757 basepairs, using a polymerase chain reaction with two primers having the nucleotide sequences of SEQ ID NO: 10 and SEQ ID NO: 11
and/or
   v) a nucleic acid sequence that can be used to amplify a nucleotide fragment of 287 basepairs, using a polymerase chain reaction with two primers having the nucleotide sequences of SEQ ID NO: 12 and SEQ ID NO: 13,
   vi) nucleic acid sequence that can be used to amplify a nucleotide fragment of 882 basepairs, using a polymerase chain reaction with two primers having the nucleotide sequences of SEQ ID NO: 14 and SEQ ID NO: 15,
   vii) a nucleic acid sequence that can be used to amplify a nucleotide fragment of 832 basepairs, using a polymerase chain reaction with two primers having the nucleotide sequences of SEQ ID NO: 16 and SEQ ID NO: 17, and/or
   viii) a nucleic acid sequence that can be used to amplify a nucleotide fragment of 9910 basepairs, using a polymerase chain reaction with two primers having the nucleotide sequences of SEQ ID NO: 18 and SEQ ID NO: 19 stably integrated into a potato plant cell nucleus.

One embodiment of the present invention provides a polynucleotide having at least 80% identity with SEQ ID NOs: 22 or 23.

A preferred embodiment according to the present invention is a polynucleotide comprising a nucleotide sequence having at least 80% identity with SEQ ID NO: 1 preferably stably integrated into a potato plant cell nucleus.

In a further embodiment, the polynucleotide comprises a nucleotide sequence having at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO: 1, 2, 3, or 44, preferably stably integrated into the genome of a potato plant cell.

In yet another embodiment, the polynucleotide comprises a nucleotide sequence having at least 80%, at least 85%, at least 90%, at least 91%, at least 92%, at least 93%, at least 94%, at least 95%, at least 96%, at least 97%, at least 98%, at least 99%, or 100% sequence identity to SEQ

ID NO: 1, 2, 3, 44 and/or 45 and comprising the blb1 and the blb2 genes, stably integrated into the genome of a potato plant cell. The polynucleotide can also further comprise one or more of SEQ ID NO: 4, 5, 6, 7, 8, 9, 10, 11, 12, 13, 14, 15, 16, 17, 18, and/or 19 in the regions flanking the inserts.

In one embodiment, the polynucleotide comprises a nucleotide sequence having at least 80% identity to SEQ ID NO: 1, 44 and/or 45 and comprise the blb1 and the blb2 genes and one or more of SEQ ID NO: 4, 5, 6, 7, 8, 9, 10, 11, 12, 13, 14, 15, 16, 17, 18, and/or 19 in the regions flanking the inserts.

“Polynucleotides” according to the present invention may be isolated polynucleotides and/or recombinant polynucleotides. Recombinant polynucleotides or recombinant construct mean any polynucleotide produced by gene technology modification e.g. by man. The gene technology modification may be transforming a plant cell with a nucleotide sequence preferably using agrobacteria.

“Identity” between two nucleic acids and/or refers in each case over the entire length of the nucleic acids.

For example the identity may be calculated by means of the Vector NTI Suite 7.1 program of the company Informax (USA) employing the Clustal Method (Higgins D G, Sharp P M. Fast and sensitive multiple sequence alignments on a microcomputer. Comput Appl. Biosci. 1989 April; 5(2):151-1) with the following settings:

Multiple alignment parameter:

| | |
|---|---|
| Gap opening penalty | 10 |
| Gap extension penalty | 10 |
| Gap separation penalty range | 8 |
| Gap separation penalty | off |
| % identity for alignment delay | 40 |
| Residue specific gaps | off |
| Hydrophilic residue gap | off |
| Transition weighing | 0 |

Pairwise alignment parameter:

| FAST algorithm | on |
|---|---|
| K-tuple size | 1 |
| Gap penalty | 3 |
| Window size | 5 |
| Number of best diagonals | 5 |

Alternatively the identity may be determined according to Chenna, Ramu, Sugawara, Hideaki, Koike, Tadashi, Lopez, Rodrigo, Gibson, Toby J, Higgins, Desmond G, Thompson, Julie D. Multiple sequence alignment with the Clustal series of programs. (2003) Nucleic Acids Res 31 (13):3497-500, the web page: http://www.ebi.ac.uk/Tools/clustalw/index.html and the following settings

| | |
|---|---|
| DNA Gap Open Penalty | 15.0 |
| DNA Gap Extension Penalty | 6.66 |
| DNA Matrix | Identity |
| Protein Gap Open Penalty | 10.0 |
| Protein Gap Extension Penalty | 0.2 |
| Protein matrix | Gonnet |
| Protein/DNA ENDGAP | −1 |
| Protein/DNA GAPDIST | 4 |

All the nucleic acid sequences mentioned herein can be produced in a known way by chemical synthesis from the nucleotide building blocks, e.g. by fragment condensation of individual overlapping, complementary nucleic acid building blocks of the double helix. Chemical synthesis of oligonucleotides can, for example, be performed in a known way, by the phosphoamidite method (Voet, Voet, 2nd edition, Wiley Press, New York, pages 896-897). The accumulation of synthetic oligonucleotides and filling of gaps by means of the Klenow fragment of DNA polymerase and ligation reactions as well as general cloning techniques are described in Sambrook et al., 1989 Molecular Cloning, Second Edition, Cold Spring Harbor Laboratory.

Sequence identity may be optimized by sequence comparison and alignment algorithms known in the art (see Gribskov and Devereux, Sequence Analysis Primer, Stockton Press, 1991, and references cited therein) and calculating the percent difference between the nucleotide sequences by, for example, the Smith-Waterman algorithm as implemented in the BESTFIT software program using default parameters (e.g., University of Wisconsin Genetic Computing Group). At least 80% sequence identity, preferably at least 85% sequence identity, especially preferred at least 90%, at least 95%, at least 98%, at least 99% sequence identity, or even 100% sequence identity, with the nucleic acid having SEQ ID NOs: 1, 2, 3, 20, 21, 22, 23, 44 and/or 45 is preferred.

The recombinant construct may encompass nucleotides having nucleic acid substitutions, deletions and/or insertions relative to the unmodified nucleic acid in question, wherein the protein coded by such nucleic acids has similar or higher functional activity as the unmodified protein coded by the unmodified nucleic acid from which they are derived. In the substitutions may be based on the degenerative amino acid code.

A “deletion” refers to removal of one or more amino acids from a protein or to the removal of one or more nucleic acids from DNA.

An “insertion” refers to one or more nucleic acid residues being introduced into a predetermined site in the nucleic acid.

Methods for the manipulation of DNA sequences to produce substitution, insertion or deletion variants of a protein are well known in the art. For example, techniques for making substitution mutations at predetermined sites in DNA are well known to those skilled in the art and include M13 mutagenesis, T7-Gene in vitro mutagenesis (USB, Cleveland, Ohio), QuickChange Site Directed mutagenesis (Stratagene, San Diego, Calif.), PCR-mediated site-directed mutagenesis or other site-directed mutagenesis protocols.

As used herein, the term “recombinant construct” refers to an expression cassette having at least 80% identity with SEQ ID NO: 2 and/or 3. In one embodiment homologues of the expression cassette have at the DNA level at least 80%, preferably of at least 90%, especially preferably of at least 95%, quite especially preferably of at least 98%, at least 99%, or 100% identity over the entire DNA region of SEQ ID NO:. 2 and/or 3. Preferably, the recombinant construct comprises the blb1-gene including the blb1 promotor and the blbl-1-terminator as well as the blb2-gene including the blb2-promotor and the blbl-2-terminator as defined below as well as a mutated ahas-gene including the p-nos-promotor and the t-nos-promotor, which are preferably capable to express the blb1 and blb2 gene and optionally the ahas gene. Said recombinant construct may be introduced in a plant cell by gene technological methods e.g. agrobacteria transformation.

In one embodiment, the recombinant construct or expression cassette or transgenic plant comprises the nucleotide sequence of SEQ ID NO: 1, 2, 3, 44 and/or 45.

As used herein, the term “blb1-gene” refers to a gene having at least 80% identity with SEQ ID NO: 46. In one embodiment homologues of the blb1-gene have at the DNA level at least 90%, preferably of at least 95%, especially preferably of at least 98%, at least 99%, or 100% identity over the entire DNA region of SEQ ID NO: 46.

As used herein, the term “blb2-gene” refers to a gene having at least 80% identity with SEQ ID NO: 47. In one embodiment homologues of the blb2-gene have at the DNA level at least 90%, preferably of at least 95%, especially preferably of at least 98%, at least 99%, or 100% identity over the entire DNA region of SEQ ID NO: 47.

As used herein, the term “mutated ahas-gene” refers to a gene having at least 80% identity with SEQ ID NO: 48. In one embodiment homologues of the mutated ahas-gene have at the DNA level at least 90%, preferably of at least 95%, especially preferably of at least 98%, at least 99% or 100% identity over the entire DNA region of SEQ ID NO: 48.

Preferably, the Phythophthora-resistant transgenic potato plant, seed, tuber, plant cell or tissue thereof expresses a functional protein corresponding to the blb1- and blb2-genes and optionally the ahas gene. Preferably, the Phythophthora-resistant transgenic potato plant, seed, tuber, plant cell or tissue thereof expresses SEQ ID NOs: 46 and 47 and optionally SEQ ID NO: 48 and/or the corresponding protein (cf. FIG. 2h).

The transgenic plant, seed, tuber, plants cell or tissue according to the present invention have a Phytophthora-resistance compared to the wildtype plant.

The wild type plant is a plant of a similar, more preferably identical, genotype as the transgenic plant having increased resistance to the Phytophthora-resistance, but does not comprise a recombinant nucleic acid comprising the blb1-gene and blb2-gene preferably regulated by their respective natural promotors and terminators.

As used herein the term “Phytophthora-resistance” or “Phytophthora-resistant”, means reducing or preventing an infection with Phytophthora infestans. Phytophthora-resistance does not imply that the plant necessarily has 100% resistance to said infection. In preferred embodiments, the resistance to infection Phytophthora infestans in a resistant plant is greater than 10%, 15%, 20%, 25%, 30%, 35%, 40%, 45%, 50%, 55%, 60%, 65%, 70%, 75%, 80%, 85%, 90%, or 95% in comparison to a wild type plant that is not resistant to Phytophthora infestans.

The terms “Phytophthora-resistance” as used herein refers to the ability of a plant, as compared to a wild type plant, to avoid infection by Phytophthora infestans, to be killed by Phytophthora infestans, to hamper, to reduce, to delay, to stop the development, growth and/or multiplication of Phytophthora infestans. The level of Phytophthora infestans resistance of a plant can be determined in various ways, e.g. by scoring/measuring the infected leaf area in relation to the overall leaf area. Another possibility to determine the level of resistance is to count the number of Phytophthora infestans colonies on the plant or to measure the amount of spores produced by these colonies. Another way to resolve the degree of fungal infestation is to specifically measure the amount of Phytophthora infestans by quantitative (q) PCR. (e.g. Llorente et al (2010) A quantitative real-time PCR method for in planta monitoring of Phytophthora infestans growth. Lett Appl Microbiol. 51(6):603-10.)

Furthermore, the transgenic plant, seed, tuber, plants cell or tissue according to the present invention provides a “comparable yield” compared to the wildtype plant, seed, tuber, plants cell or tissue.

The term “comparable yield” as used herein refers to the ability of the transgenic potato plant compared to wildtype plant to provide a similar amount of tubers. A similar amount of tubers means that the relative yield of transgenic tubers based on the yield/ha of wildtype tubers (kg/ha) is at least 95% , preferably at least 96%, at least 97%, at least 98%, at least 99% of the yield/ha of the wildtype tubers or more preferably the same or more than the yield/ha of the wildtype tubers.

The %-relative yield is calculated as follows:

%=transgenic tubers (kg/ha)×100%/wildtype tubers (kg/ha).

The term “plant” is intended to encompass plants at any stage of maturity or development, as well as any tissues or organs (plant parts) taken or derived from any such plant unless otherwise clearly indicated by context. Plant parts include, but are not limited to, plant cells, stems, roots, flowers, ovules, stamens, seeds, leaves, embryos, meristematic regions, callus tissue, anther cultures, gametophytes, sporophytes, pollen, microspores, protoplasts, hairy root cultures, and/or the like. As used herein, a “plant cell” includes, but is not limited to, a protoplast, gamete producing cell, and a cell that regenerates into a whole plant. Tissue culture of various tissues of plants and regeneration of plants there from is well known in the art and is widely.

The present invention also includes seeds produced by the plants of the present invention. Preferably, the seeds comprise a nucleic acid sequence having at least 80% identity with SEQ ID NO:1. The generated transformed plants may be propagated by clonal propagation or classical breeding techniques.

For the purposes of the invention, “recombinant construct” or “recombinant nucleic acid” means an expression cassette or a vector construct comprising the blb1-gene and the blb2-gene in combination with their natural promoters and terminators. Said expression cassette comprising the blb1-gene, blb2-gene, blb1-promotor, blb2-promotor, blb1-terminator and blb2-terminator are defined above.

As used herein, the term “transgenic” preferably refers to any plant, plant cell, tuber, callus, plant tissue, or plant part that contains the recombinant construct or a part thereof which is preferably introduced by non-essentially biological processes, preferably agrobacteria transformation. The recombinant construct or a part thereof is stably integrated into a chromosome, so that it is passed on to successive generations by clonal propagation, vegetative propagation or sexual propagation. Said successive generations are also transgenic. Essentially biological processes may be crossing of plants and/or natural recombination.

A transgenic potato plant, seed tuber, plants cell or tissue for the purposes of the invention is thus understood as meaning that the recombinant cassette integrated into the genome. Preferably, the blb1-gene and/or the blb2-gene and/or the ahas-gene are not present in the genome of the original plant and preferably are present in the genome of the transgenic plant not at their natural locus of the genome of the original plant.

Natural locus means the location on a specific chromosome, preferably the location between certain genes, more preferably the same sequence background as in the original plant which is transformed.

Preferably, the transgenic potato plant, seed tuber, plants cell or tissue thereof expresses the blb1-gene and the blb2-gene. The term “expression” or “express” means the transcription of a specific gene or specific genes or specific genetic vector construct. The term “expression” or “express” in particular means the transcription of a gene or genes or genetic vector construct into structural RNA (rRNA, tRNA) or mRNA with preferably a subsequent translation of the latter into a protein.

The term “increased expression” or “overexpression” or “increase of content” as used herein means any form of expression that is additional to the original wild-type expression level. For the purposes of this invention, the original wild-type expression level might also be zero (absence of expression or absence of respective gene(s)).

The wildtype plant cells may be transformed with one of the above described recombinant construct. Suitable methods for transforming host cells including plant cells are well known in the art of plant biotechnology. Any method may be used to transform the recombinant expression vector into plant cells to yield the transgenic plants of the invention. The wildtype plants cells may be e.g. from Fontane, Agria, Bientje, Sarpo Mira, Cara, Valor, Innovator, Diamant.

Transformation can also be carried out by bacterial infection by means of Agrobacterium (for example EP 0 116 718), viral infection by means of viral vectors (EP 0 067 553; U.S. Pat. No. 4,407,956; WO 95/34668; WO 93/03161) or by means of pollen (EP 0 270 356; WO 85/01856; U.S. Pat. No. 4,684,611). Agrobacterium based transformation techniques are well known in the art. The Agrobacterium strain (e.g., Agrobacterium tumefaciens or Agrobacterium rhizogenes) comprises a plasmid (Ti or Ri plasmid) and a T-DNA element which is transferred to the plant following infection with Agrobacterium. The T-DNA (transferred DNA) is integrated into the genome of the plant cell. The T-DNA may be localized on the Ri- or Ti-plasmid or is separately comprised in a so-called binary vector. Methods for the Agrobacterium-mediated transformation are described, for example, in Horsch RB et al. (1985) Science 225:1229. The transformation of potatoe by Agrobacteria is described in, for example WO 2008/R34876). Transformation may result in transient or stable transformation and expression. Although a nucleotide sequence of the present invention can be inserted into any plant and plant cell falling within these broad classes, it is particularly useful in potato plant cells.

The genetically modified plant cells can be regenerated via all methods with which the skilled worker is familiar. Suitable methods can be found in the abovementioned publications by S. D. Kung and R. Wu (White F F, Vectors for Gene Transfer in Higher Plants, Transgenic Plants, Vol. 1, Engineering and Utilization, edited by S. D. Kung and R. Wu, Academic Press, 1993, pp. 15-38; Jenes B et al. Techniques for Gene Transfer, Transgenic Plants, Vol. 1, Engineering and Utilization, edited by S. D. Kung and R. Wu, Academic Press, 1993, pp. 128-143) Potrykus (Potrykus (1991) Annu Rev Plant Physiol Plant Molec Biol 42:205- 225.) or or Höfgen and Willmitzer (Höfgen R, Willmitzer L (1988) Storage of competent cells for Agrobacterium transformation. Nucleic Acids Res 16:9877).

The recombinant construct may comprise a mutated ahas-gene as a selection marker. Plants carrying the construct are resistant to immidazolines. For selection of transgenic potato plants chemical compounds inhibiting the AHAS enzyme can be used. Useful compounds are the imidazoline type herbicides. Especially useful compounds are selected from the group consisting of imazethapyr (Pursuit®), imazamox (Raptor®), imazamethabenz (Assert®), imazapyr (Arsenal®), imazapic (Cadre®) and imazaquinon (Scepter®). For selection of transgenic plants chemical compounds as described in the review article by Duggleby, R. G. and Pang, S. S. in Journal of Biochemistry and Molecular Biology 33(1), 1-36 (2000) can be used.

The transformed plant tissue may be exposed to 0.5 μM imazamox such selecting the plant material carrying the construct.

Following DNA transfer and regeneration, putatively transformed plants may also be evaluated, for instance using Southern analysis, for the presence of the whole recombinant construct, copy number and/or genomic organisation.

Gene targeting in plants is possible, but it is a quite rare event (Hanin & Paszkowski 2003 Current Opinion Plant Biol. 6(2):157-62). However, the person skilled in the art will know how to improve gene targeting frequency. For example, one could increase gene targeting frequency by expressing proteins, which facilitate the process of homologous recombination such as yeast RAD54 (Shaked et al. 2005 Proc Natl Acad Sci USA 102(34):12265-9). Another approach is to facilitate detection of gene targeting lines by a strong positive-negative selection system (lida & Terada 2005 Plant Mol. Biol 59: 205-219). In such approach a negative selectable marker is located outside of the homologous sequences on the transformation construct. In consequence, only those transgenic plants with random insertion of the transgenic sequences contain the negative selectable marker, while transgenic lines obtained through gene targeting do not comprise the negative selectable marker.

Furthermore, gene targeting frequency can be drastically increased by introducing a DNA double strand break at or near the desired insertion site. The person skilled in the art will know how to achieve this. For example, natural occurring homing endonucleases (also referred to as meganucleases, e.g. I-Crel) can be modified such that they recognize and cut a novel DNA sequence, i.e. the sequence at or near the desired insertion site in the genome (WO 07/047859, WO 07/049156). Alternatively, one could design so called zink finger nucleases, which are comprised of a unspecific nuclease domain (usually obtained from FokI nuclease) linked to a zink finger, which specifically recognizes the desired DNA sequence (compare for example Trends Biotechnol. 2005 23(12):567-9; Cell Mol Life Sci. 2007 64(22):2933-44; WO 08/021207). Another method is the usage of TAL (transcription activator like) effectors linked to a DNA specific nuclease (e.g. Fok1) as described in Mahfouz et al. 2011. De novo-engineered transcription activator-like effector (TALE) hybrid nuclease with novel DNA binding specificity creates double-strand breaks; PNAS 108(6)2623-2628. By using this method, a TAL effector variant binding to the desired target sequence can be easily generated by changing the well defined aminoacids binding to the DNA (the code can be found in WO/2010/079430).

Gene targeting may be used to obtain a line similar to the elite-event D by inserting a transgenic construct comprising the recombinant construct at essentially the same insertion site as found in elite-event D. The person skilled in the art will know that the insertion site may differ in a few base pairs or up to a few kilo base pairs, but still obtaining a similar line with similar beneficial characteristics. Gene targeting may in particular be used to establish a line similar to elite-event

D in a potato variety other than Fontane. It may be of interest to establish such a corresponding line based on other varieties more particularly suited for environmental conditions found in different potato growing regions.

### FIGURES

FIG. 1: Vector card VCPMA 16

FIG. 2: Sequences of the present application

FIG. 3: Overview of primers

FIG. 4: Chart comparing the relative yield of events A to D, Bintje (standard variety) with Fontane (mother variety of events A to D).The average yield/ha of Fontane, events A-D and Bintje was measured over 3 years at 15 locations in the field. The average yield/ha of the Fontane variety was set to 100% and relative yields of events A-D and Bintje were calculated accordingly.

FIG. 5: Chart shows the result of Phytophthora screening of Fontane compared with events A to D and Bientje. Diseased leaf area was scored in the field after natural infection. The mother variety Fontane was set to 100%. All events show full resistance against Phytophthora infestans.

### EXAMPLES

The following examples are not intended to limit the scope of the claims to the invention, but are rather intended to be exemplary of certain embodiments. Any variations in the exemplified methods that occur to the skilled artisan are intended to fall within the scope of the present invention.

### Example 1

### General Methods

The cloning steps carried out for the purposes of the present invention such as, for example, restriction cleavages, agarose gel electrophoresis, purification of DNA fragments, transfer of nucleic acids to nitrocellulose and nylon membranes, linking DNA fragments, transformation of E. coli cells, bacterial cultures, phage multiplication and sequence analysis of recombinant DNA, are carried out as described by Sambrook et al. Cold Spring Harbor Laboratory Press (1989), ISBN 0-87969-309-6.

The chemical synthesis of oligonucleotides can be affected, for example, in the known fashion using the phosphoamidite method (Voet, Voet, 2nd Edition, Wiley Press New York, pages 896-897). The sequencing of recombinant DNA molecules is carried out with an MWG-Licor laser fluorescence DNA sequencer following the manual of the manufacturer based on the method by Sanger (Sanger et al., Proc. Natl. Acad. Sci. USA 74, 5463 (1977).

### Example 2

### Cloning of Transformation Vector VC-PMA16

pSUNAHASmod was used as backbone for the construction of VCPMA16. pSUNAHASmod is based on the plasmid pSUN1 (WO 02/00900). The T-DNA of pSUNAHASmod contains a mutated AHAS (Acetohydroxyacid-Synthase)-gene (S653N), which enhances the resistance of the transformed plant against imidazolinone herbicides (e.g. Imazamox: (R/S)-2-(4-isopropyl-4-methyl-5-oxo-2-imidazolin-2-yl)-5-methoxymethylnicotinic acid). The use of the mutated AHAS gene as selection marker is described in Andersson et al. (2003) Plant Cell Rep 22:261-267 and WO 2004/005516. The AHAS selection cassette was constructed by fusing the nos promoter fragment from pGPTVKan (Becker et al., Molecular plant Biology 20, 1195-1197), the mutated AHAS gene from Arabidopsis and the nos-terminator from pGPTVKan (Becker et al., 1992 Molecular plant Biology 20, 1195-1197).

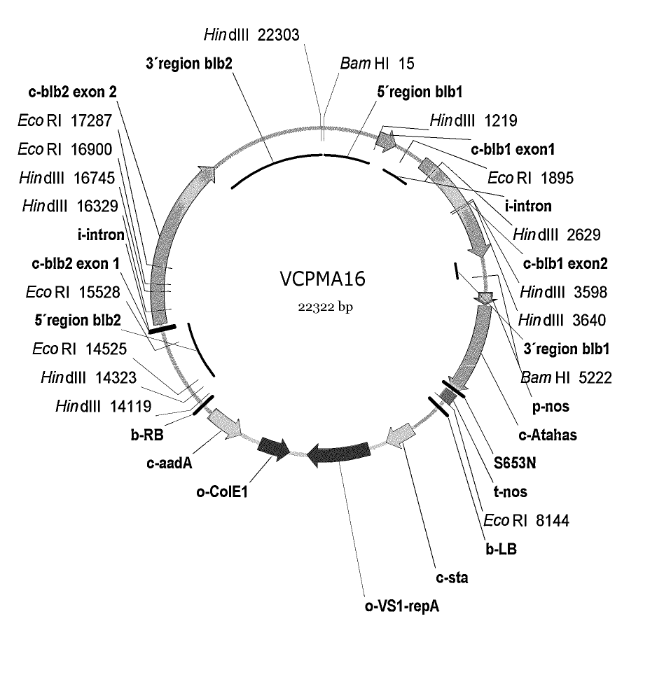

The blb1-gene fragment, including the 1173 by blb1-promotor-region and the 406 by blb1 terminator region was ligated into pSUNAHASmod by using the Xbal restriction site. The blb2-gene expression cassette comprises the Rpi-blb2 gene (3890 bp), the blb2-promoter sequence (1530 bp) and 2530 by blb2 termination sequence. To insert the blb2-expression cassette into the blb1 containing pSUNAHASmod, the vector was cut with Pstl. The resulting sticky restriction sites were blunted and the blb2 expression cassette was inserted in a blunt-blunt ligation. The resulting vector was named VCPMA16 (FIG. 1).

### Example 3

### Transformation of Agrobacterium Tumefaciens (A. Tumefaciens) with VCPMA16

The construct VCPMA16 was transformed into the A. tumefaciens strains LBA4404, AGL0 or AGL1 by using direct transformation as described by Walkerpeach & Velten (Agrobacterium-mediated gene transfer to plant cells: co-integrate and binary vector systems, Gelvin S B, Schilperoot R A (Hrsg.), Plant Molecular Biology Manual, 2nd edn, Kluwer Academic Publishers, Dordrecht, Netherlands, pp. B1/1-B1/19, 1994). Transformed bacteria were grown on YEB agar plates containing 1 μg/ml spectinomycin.

### Example 4

### Cultivation and Transformation of Potato Cultivar Fontane using A. Tumefaciens

The potato variety Fontane was transformed with VCPMA16 by using Agrobacterium mediated transformation as described by Visser (Visser RGF, 1991, “Regeneration and transformation of potato by Agrobacterium tumefaciens.” In Lindsey K (ed), “Plant Culture Manual”, Kluwer Academic Publishers, Dordrecht, Netherlands. Seiten B5/1-B5/9) but using Imazamox as selection marker (WO 2004/005516; Andersson et al., 2003, plant Cell Rep 22: 2261-267).

Potato leaf or shoot segments were incubated for 1-3 days on MC-plates (M300-Plates (4,4 g/l MS-Medium, 2 mg/l NAA, 1 mg/l BAP, 30 g/l Sucrose, pH 5.2) covered with 1.5-2 ml liquid M100-Medium (4.4g/l MS-Medium, 30 g/l Sucrose, 0,5 mg/ml Thiamin-Hydrochloride, 0.5 mg/ml Pyridoxin-Hydrochloride, 1 mg/l Nikotinic acid, 0.5 mg/l Kinetin, 29.8 mg/l FeSO₄*7H₂O, 1 mg/l 2,4-D, 2 g/l Casein-Hydrolysate, pH 6.5) and covered with a sterile filter paper.

After 1-3 days the tissue segments were incubated with A. tumefaciens (containing VCPMA16) in MS10-Medium (4.4 g/l MS-Medium, 10 g/l Sucrose, pH 5.8). After 8-10 min. the tissue segments were transferred to M300 plates (see above). After 1-3 days the tissue segments were transferred to MS400-plates (4.4 g/l MS-Medium, 2 mg/l zeatine, 0.01 mg/l NAA, 0.1 mg/l GA3, 10 g/l Sucrose, 400 mg/l Claforane or carbenicilline, pH 5.8) and incubated for another 3-5 days.

### Example 5

### Selection of the Transformed Potato Plantlets

After 3-5 days the tissue segments were transferred to MS400 plates (see above) containing 0.5 μmol Imazamox as selection agent. Every 2 weeks the tissue segments were conveyed to new MS400 plates containing 0.5 μmol Imazamox. Growing (regenerated) shoots were harvested and transferred to MS30 plates (4.4 g/l MS-Medium, 30 g/l Sucrose, 200 mg/l claforane, pH 5.8) for further cultivation.

### Example 6

### DNA Extraction from Transformed Potato Shoots

DNA was extracted from putative transgenic shoots by using the Wizard Magnetic 96 DNA Plant System (Promega, Mannheim) Kit according to the instructions of the manufacturer.

### Example 7

### Detection of Rpi-blb1 and Rpi-blb2 in Transformed Potato Plants Using Real-Time PCR

To detect the presence of blb1 and blb2 a real time PCR was performed using the DNA from putative transgenic potato shoots (see above) as a template. The following primers were used:

blb1:
(forward Primer; SEQ ID No. 49)
5′-TGT TGA ACA CTG TAA CAT GCT AAA ATG-3′
(backward Primer; SEQ ID No. 50)
5′-AGT TGT GGA CAT CCC CGA ATT-3′
(Probe; SEQ ID No. 51)
5′-AGA GGG ATT GCA GCA CCT AAC AAC CCT C-3′
blb2:
(forward Primer; SEQ ID No. 52)
5′-TTC AAA ACC CCA AAT AAG TTT CAA C-3′
(backward Primer; SEQ ID No. 53)
5′-CCA TGC TTG CTG TAC TTT GCA-3′
(Probe; SEQ ID No. 54)
5′-CGT TAC CCA GTC CTT CGG CG-3′

The samples were analyzed using a Roche Lightcycler480 by using 20-50 ng genomic DNA, 900 mM PCR primer (see above), 200 nM probe (see above) in 1× LightCycler 480 Probe Master (for detailed protocol see manual of the manufacturer).

The amplification cycles of the PCR were:

1 cycle of 15 min at 95° C. for denaturation, followed by 40 cycles of in each case 10 seconds at 95° C. (Ramp Rate 4.8° C./sec) and 30 sec at 60° C. (Ramp Rate 2,5° C. /sec).

If the PCR of both fragments resulted in a positive signal, the shoots were transferred into the greenhouse to conduct Phytophthora resistance tests.

### Example 8

### Determination of the Resistance Level of blb1 and blb2 Containing Transgenic Potato Shoots Against Phytophthora infestans

The blb1 and blb2 containing transgenic potato shoots (as determined above) were transferred into soil and adapted to soil for 2 days at 22° C. with 12 h day-length and 100% humidity in a growth cabinet (Binder KBW 400). Afterwards plants were grown in the greenhouse or phytochambers under similar conditions but 70% humidity.

After 4 weeks plants were inoculated with Phytophthora infestans spores. To prove the broad spectrum resistance mediated by blb1 and blb2, a multitude of different Phytophthora isolates, e.g. Blue13, Us-22 and many locally collected strains were tested collected from all over the world, either in mixtures or as single isolates.

All Phytophthora isolates were cultivated on pea-agar as described in Table 2.

TABLE 2
Pea Agar preparation.

| | |
|---|---|
| Pea-agar: | 150 g peas |
| | 1000 ml Millipore-ater |
| | cook for 75 min. in a steamer |
| | cool down for ~1 hour, incubate for 24 h at RT |
| | strain media and refill with 1 l Millipore water |
| | add 5 g Glucose and 20 g agar-agar |
| | adjust pH to 6.5 |
| | autoclave for 15 min |
| | pour plates under sterile conditions |

Plant were inoculated with a spore density of 2,5×E05 spores/ml. The spore density was evaluated by using a Thoma counting chamber. For inoculation the complete plant was sprayed with spore suspension and transferred into a dark mist chamber. After 12-18 hours the plants were moved to the greenhouse (21° C., 12 h light, >90% humidity) for one week. First disease symptoms occurred after approx. 1 week. The rating of disease symptoms was done by trained personal evaluating the diseased leaf area, necrotic lesions, chlorotic lesions and potential sporulation of P. infestans.

These values were integrated into a disease rating ranging from 0 to 100%. In the scoring system 0% disease means no macroscopically visible symptoms, whereas 100% means that all inoculated leaves are completely brownish and covered with mycelia, so the plant is essentially dead. Inoculation of the susceptible mother variety Fontane potato variety generally leads to a strong infection of all leaves. All leaves are heavily infected and green tissue is rare. In contrast the inoculation of the transformed Fontane Event A to D always leads completely healthy plants with a disease rating of 0% for all used Phytophthora isolates. As susceptible control the standard variety Bintje was used, which is known to be fully susceptible to Phytophthora infection.

### Example 9

### DNA Isolation and Quantitation Methods for FST Identification

Young leaf tissue of the fungal resistant potato events were collected for DNA isolation and characterization. Upon collection, the leaf tissues were frozen with liquid nitrogen and lyophilized.

DNA was isolated from potato leaf tissue using a modified cetyl trimethyl ammonium bromide (CTAB) method (Carlson et al., 1991). Dry leaf tissue was ground with a pestle and a mortar. The ground tissue was incubated with preheated extraction buffer consisting of 2% (w/v) CTAB, 100 mM Tris-HCl, 1.4 M NaCl, 1% (w/v) polyvinylpyrrolidone (PVP), 20 mM ethylenediamine tetraacetic acid (EDTA), pH 9.5 (5 ml/l g fresh leaf tissue) and beta-mercaptoethanol (2.5 μl/ml buffer) at 74° C. for 20 min. After centrifugation at 2440×g for 10 min, the supernatant was extracted twice with an equal volume of chloroform/isoamyl alcohol (24:1). DNA was precipitated with 0.7 volume of isopropanol and dissolved in TE buffer (10 mM Tris-HCl, 1 mM EDTA, pH 8.0) with 0.5 mg/ml RNase A (Invitrogen; Carlsbad, Calif. 92008 USA) added to a final concentration of about 500 ng/μl. The isolated DNA was quantified with Hoechst 33258 dye (Invitrogen) using calf thymus DNA (Invitrogen) as the DNA standard on an FLx800™ Microplate Reader (BioTek Instruments, Winooski, Vt. 05404, USA) according to the fluorometer user manual.

### Example 10

### Tail PCR Amplification of Flanking Sequences

Oligonucleotide primers. T-DNA specific primers which are complementary to the AHAS coding sequence and the Blb2 promoter region in VC-PMA16, respectively, were synthesized (Table 3).

TABLE 3
T-DNA specific primers for cloning of flanking sequences using tail PCR

| | | Position | |
|---|---|---|---|
| | | in VC- | |
| Name | Sequence | PMA16 | Comment |
| 07-038_P25 | AACGATGTCATAACGGAAGG | 16136 | Specific primer for tail PCR |
| | (SEQ ID NO: 55) | | (LB1) |
| 07-038_P26 | AGAGCATTTGAAGCAGATCTAGGGT | 464 | Specific primer for tail PCR |
| | (SEQ ID NO: 56) | | (RB1) |
| 07-038_P27 | CGGATTAAATACTGAGAGCTCGAAT | 16163 | Specific primer for tail PCR |
| | (SEQ ID NO: 57) | | (LB2) |
| 07-038_P28 | CAGATCTAGGGTTTTATCTCGG | 454 | Specific primer for tail PCR |
| | (SEQ ID NO: 58) | | (RB2) |
| 07-038_P29 | TGCCGGTCTTGCGATGATTA | 16241 | Specific primer for tail PCR |
| | (SEQ ID NO: 59) | | (LB3) |
| 07-038_P30 | AGATCTAGGGTTTTATCTCGGGATT | 450 | Specific primer for tail PCR |
| | (SEQ ID NO: 60) | | (RB3) |

LB = left flanking primer,
RB = right flanking primer

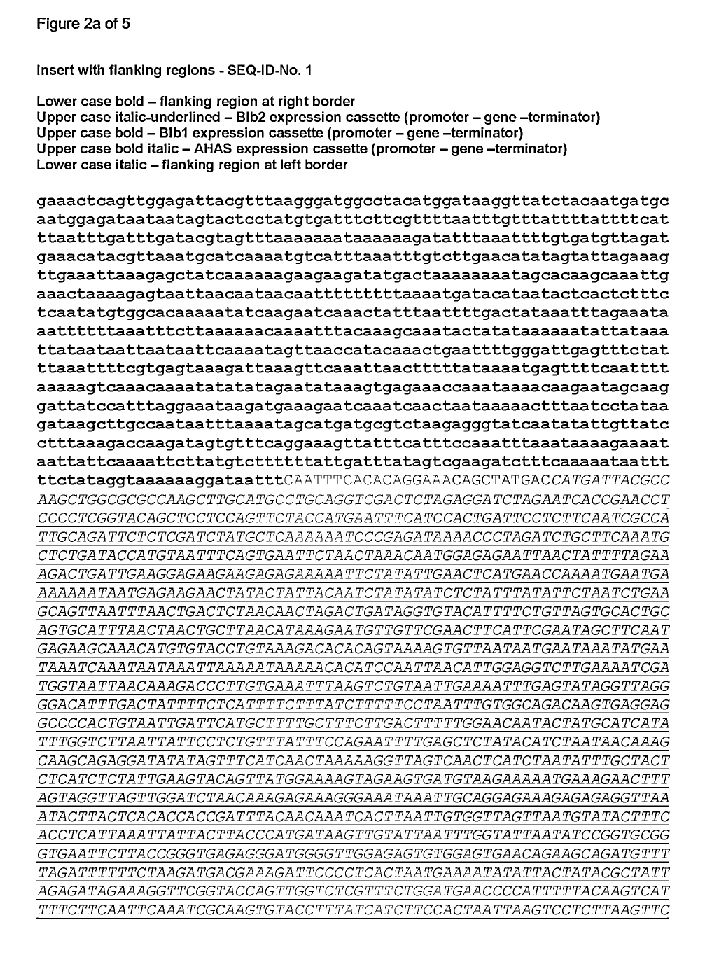

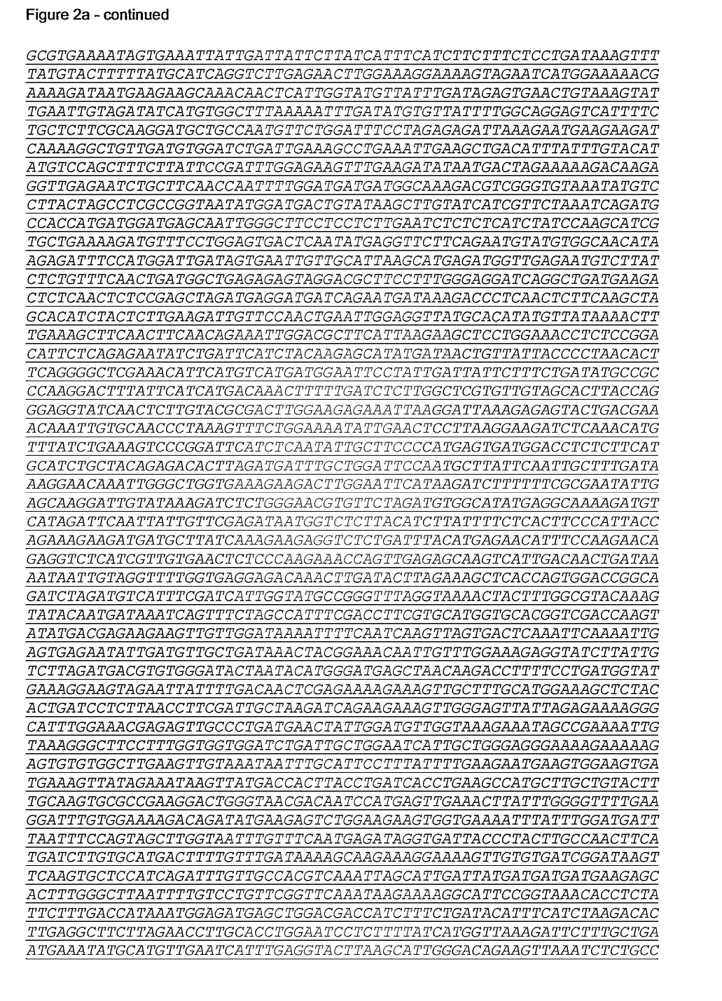

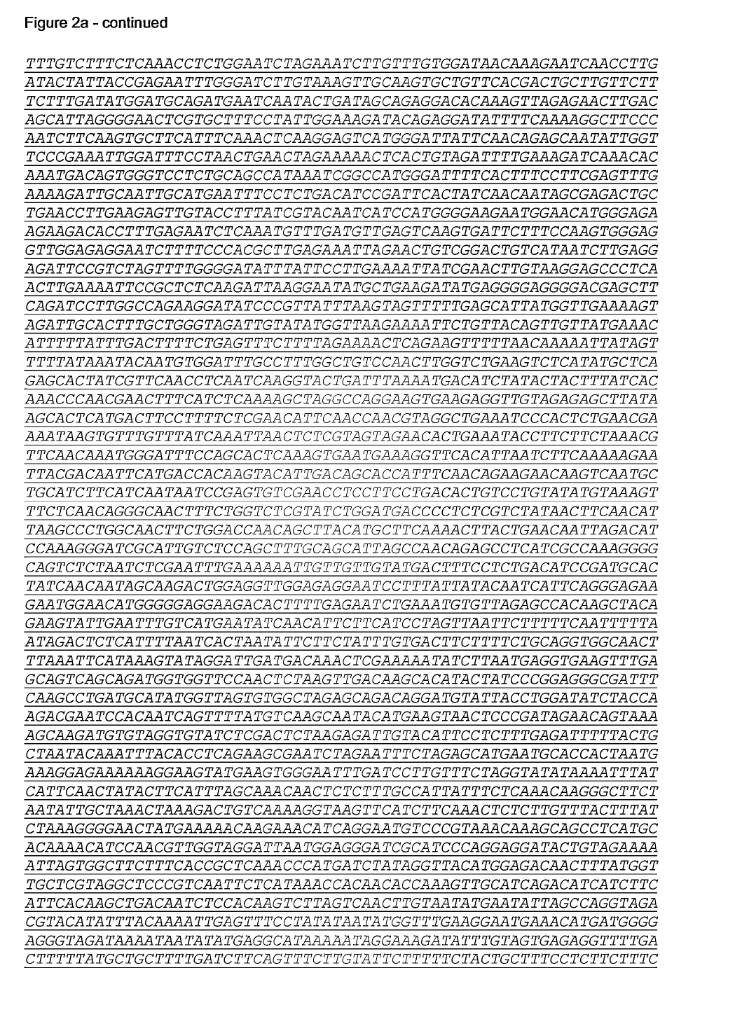

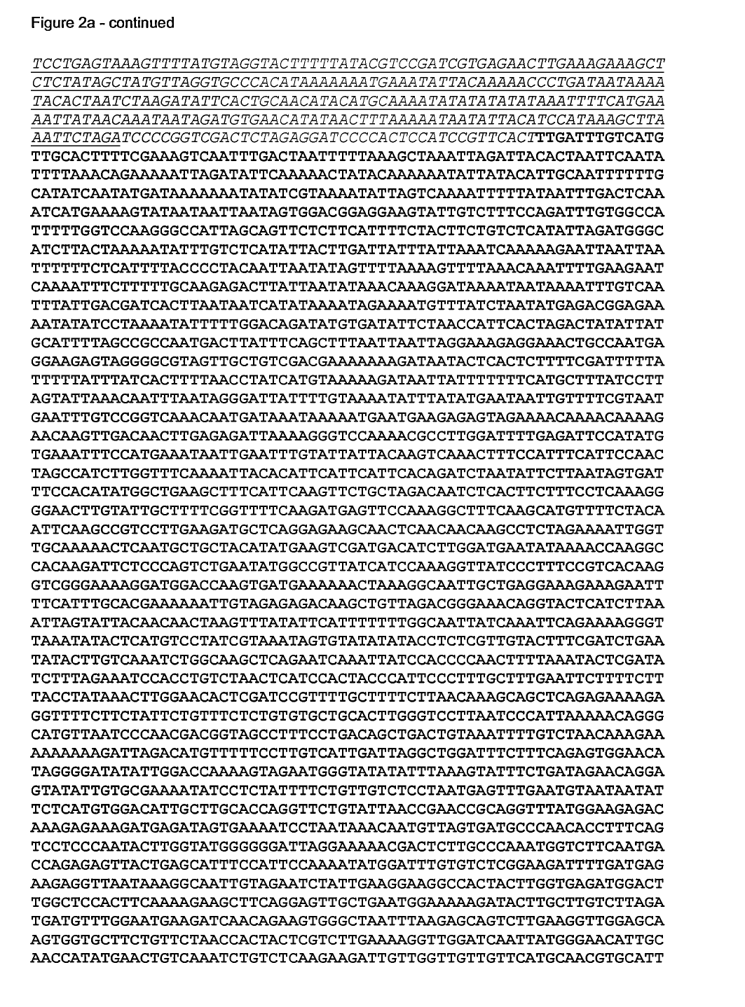

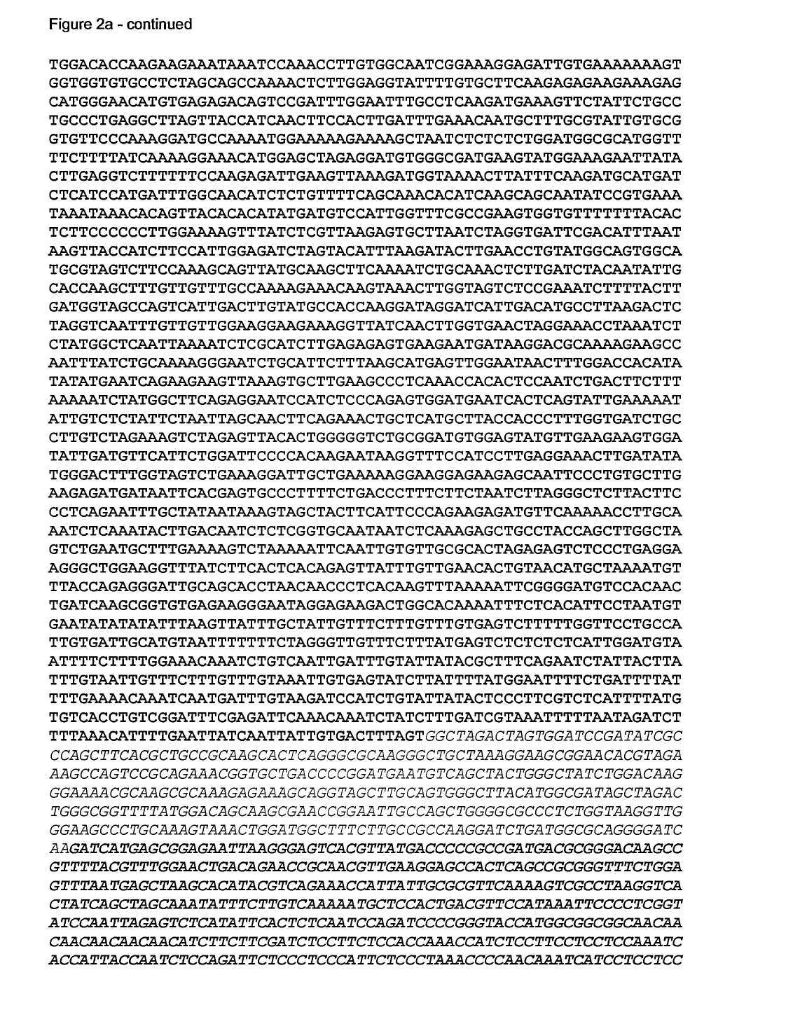

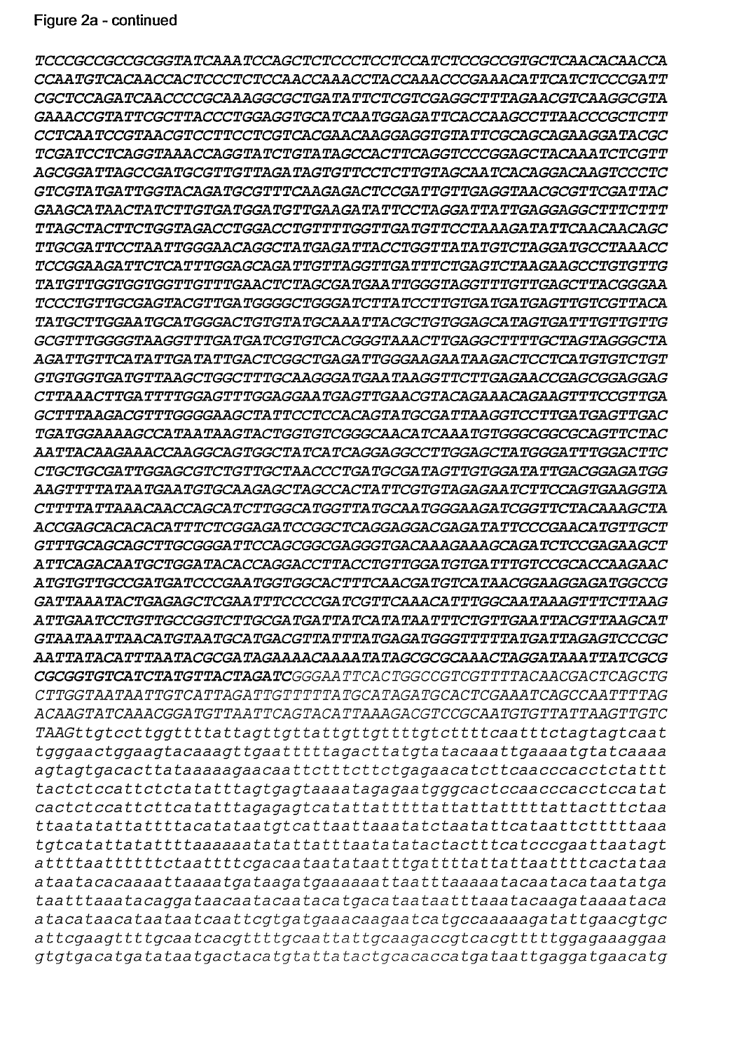

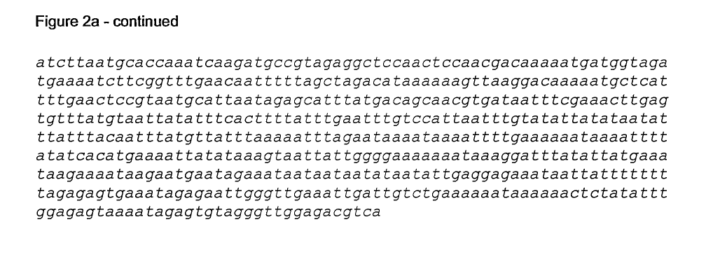

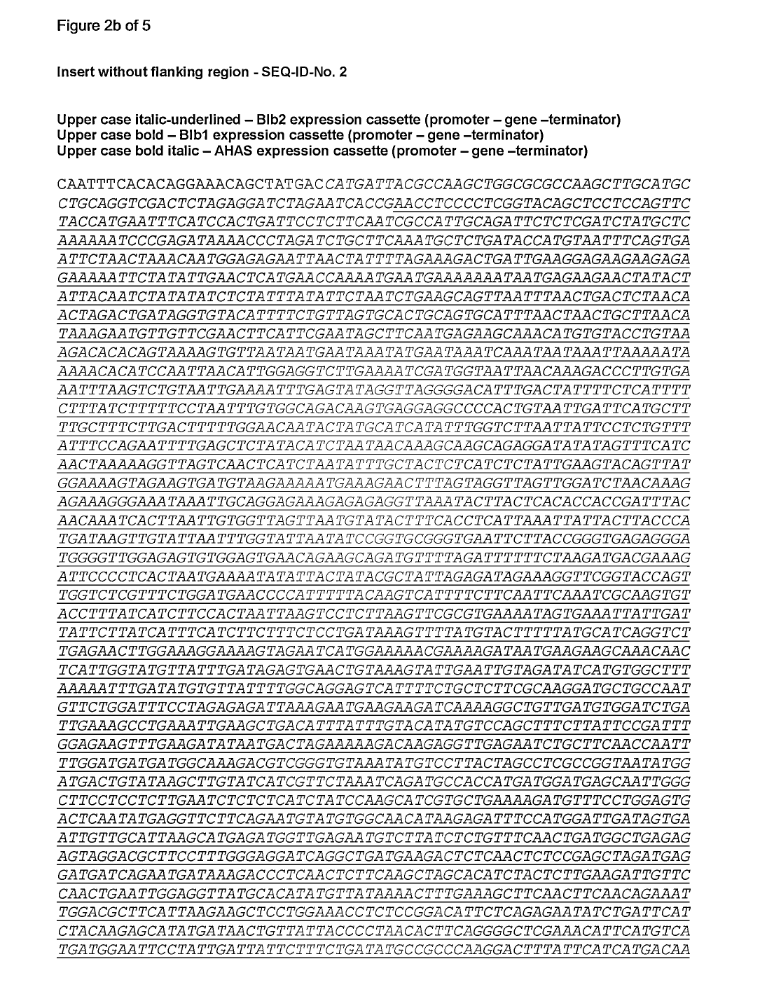

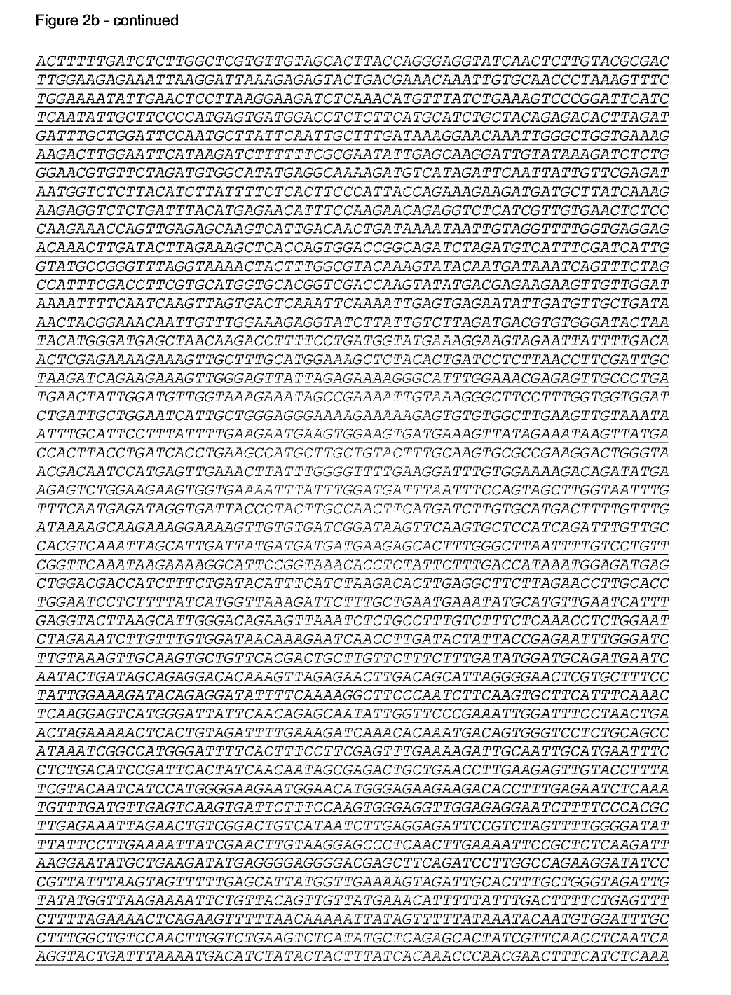

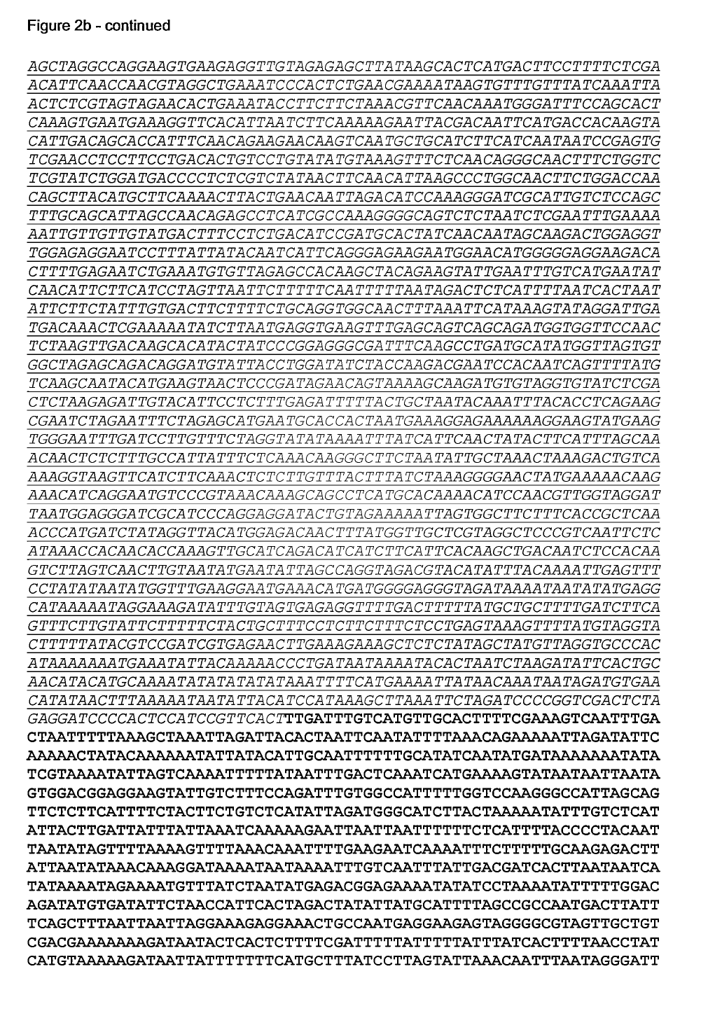

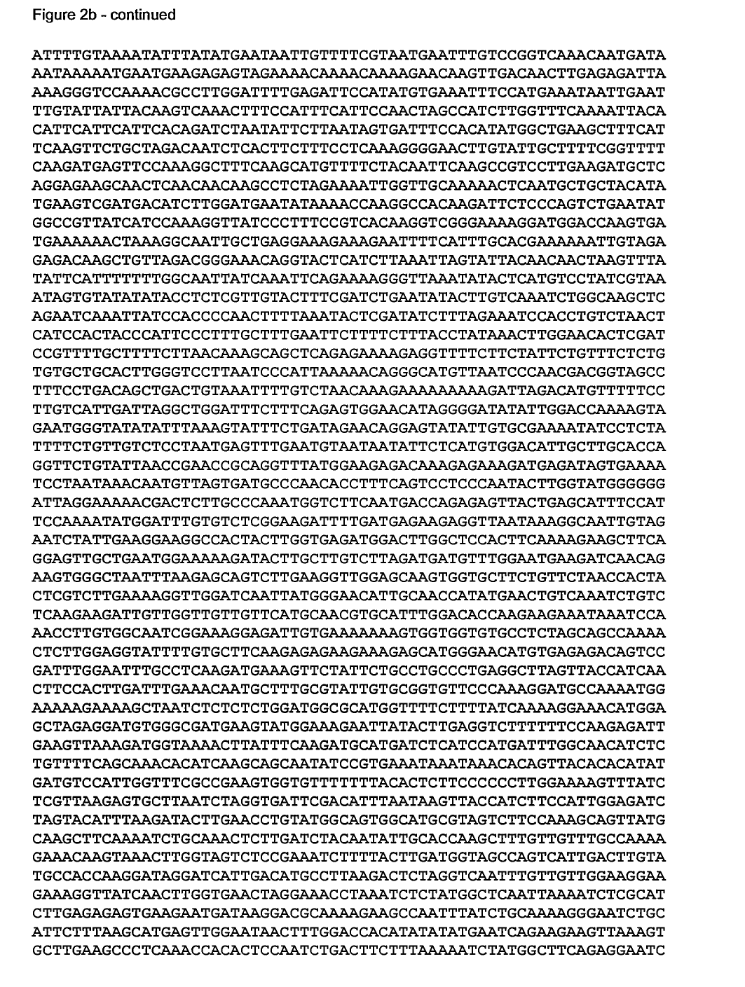

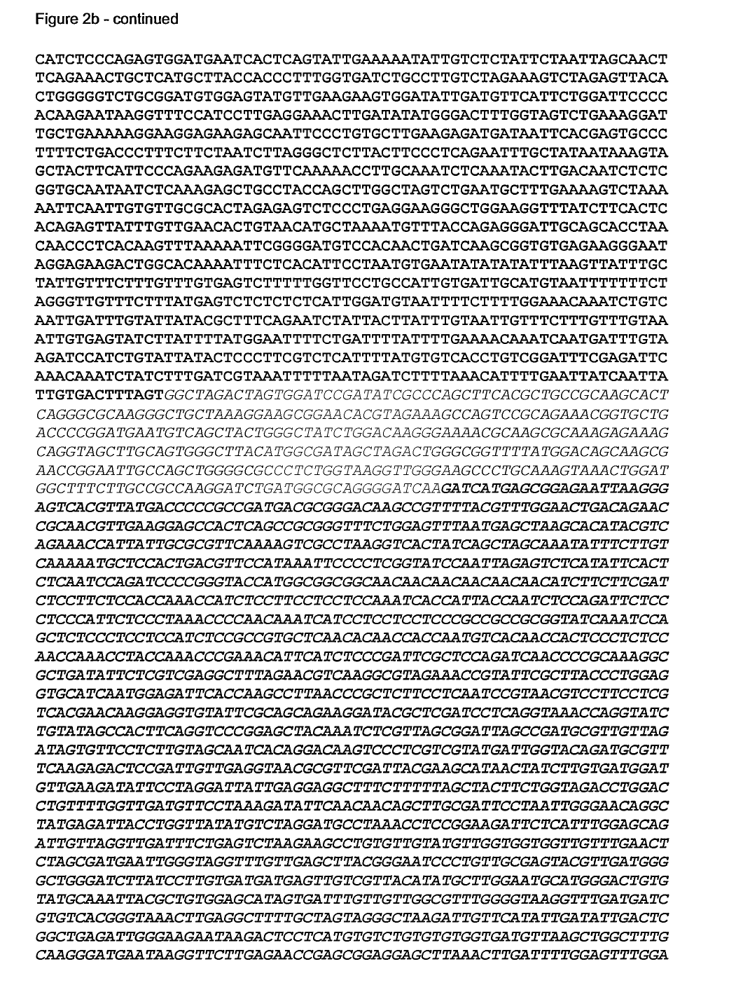

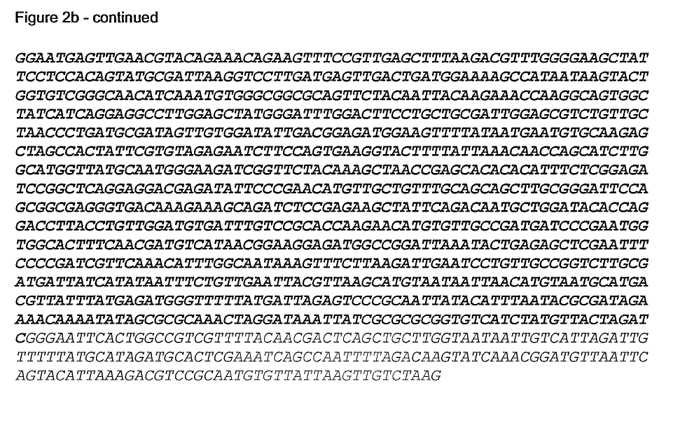

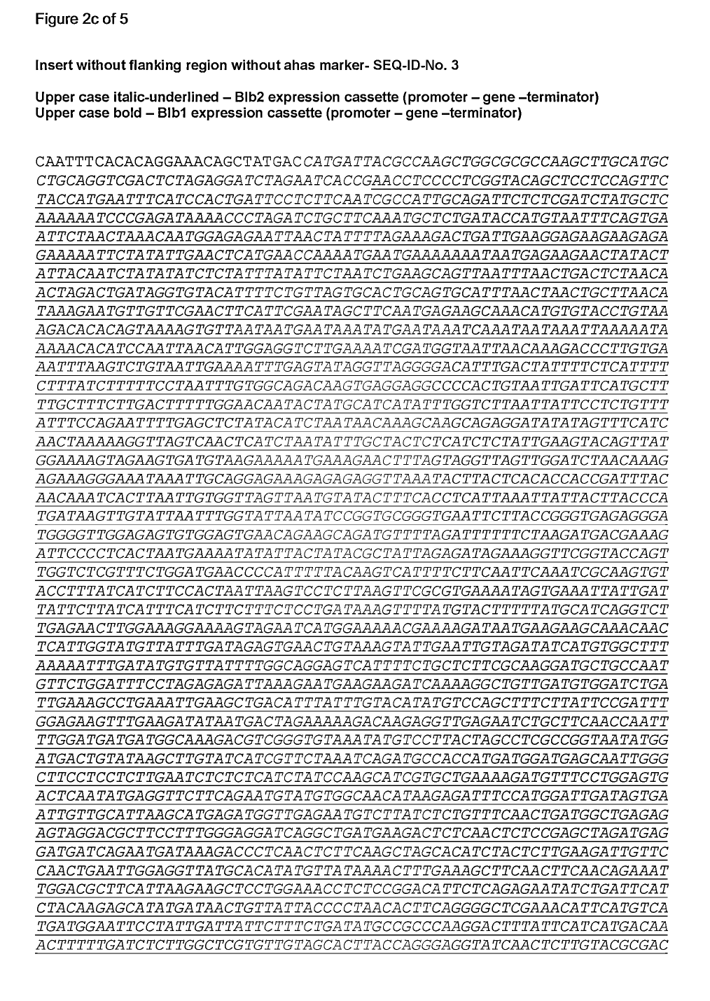

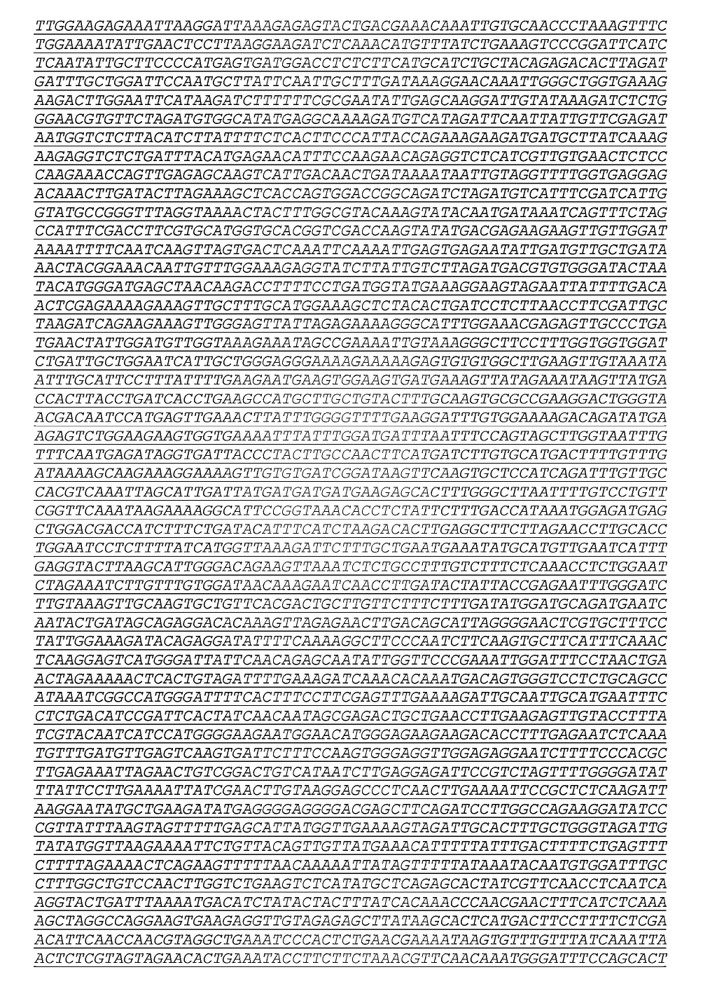

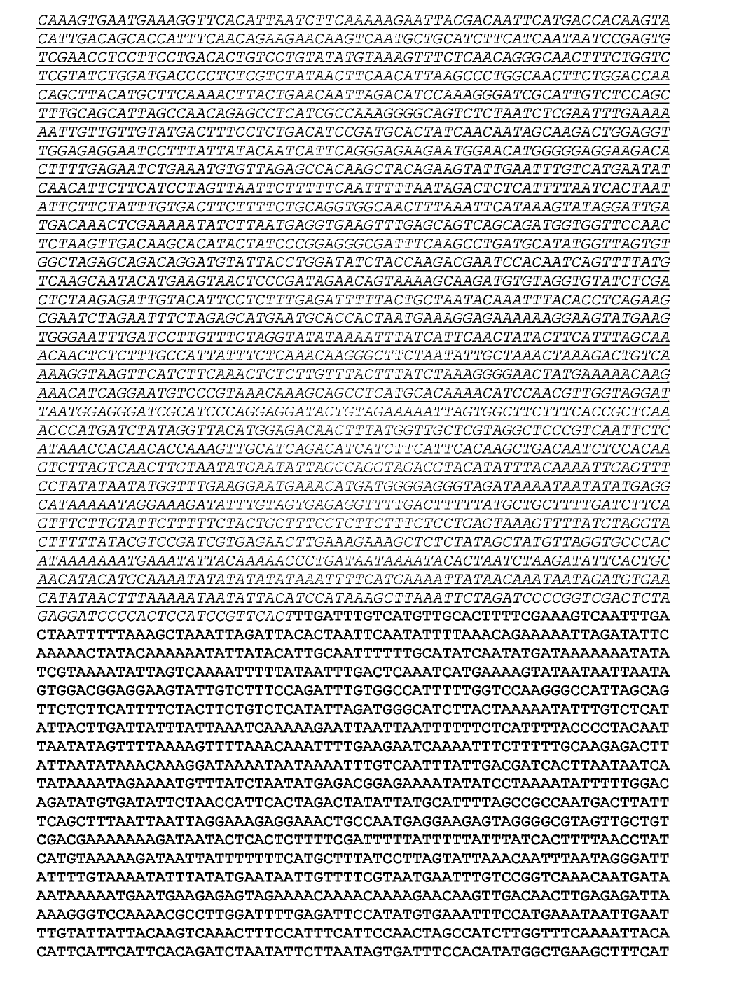

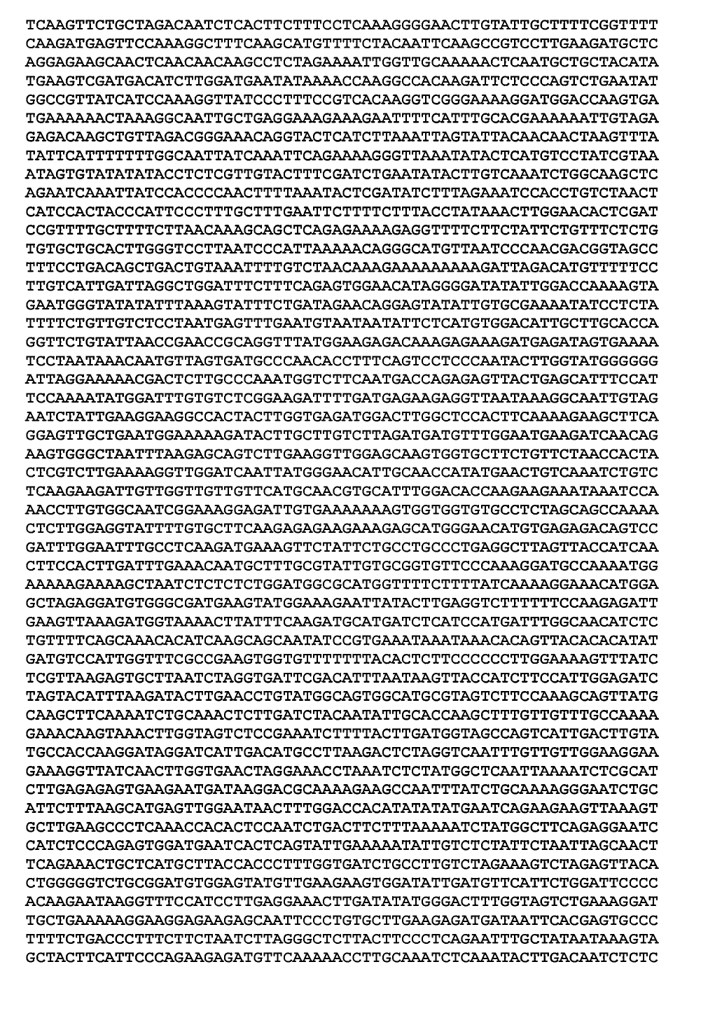

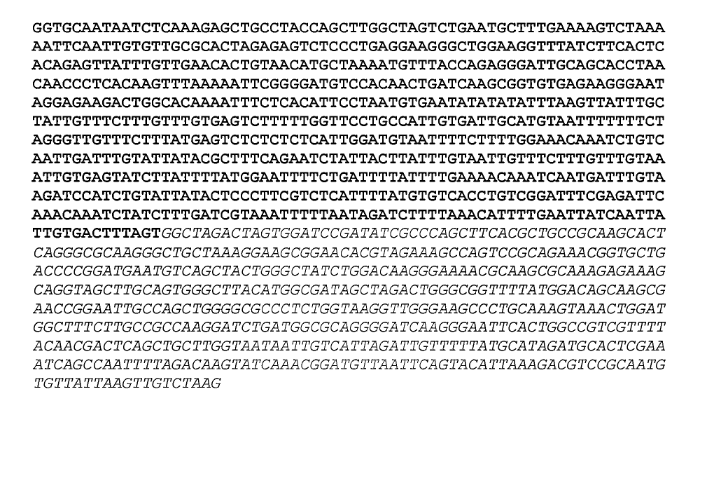

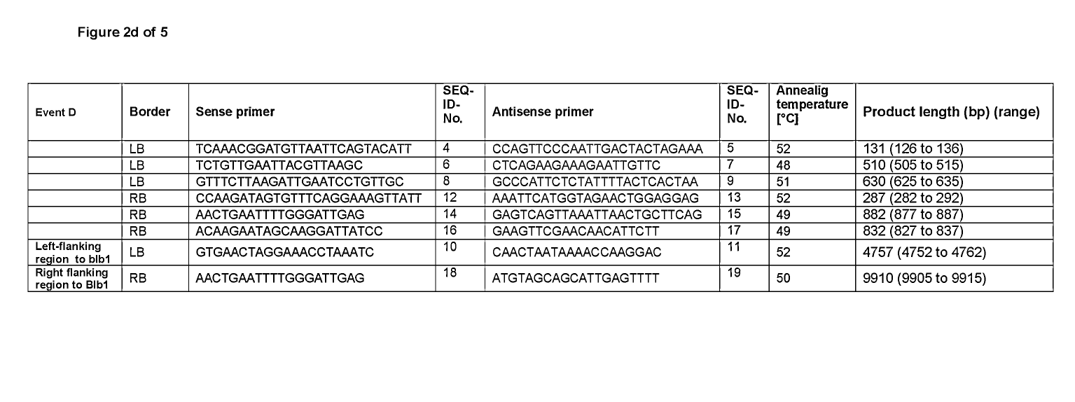

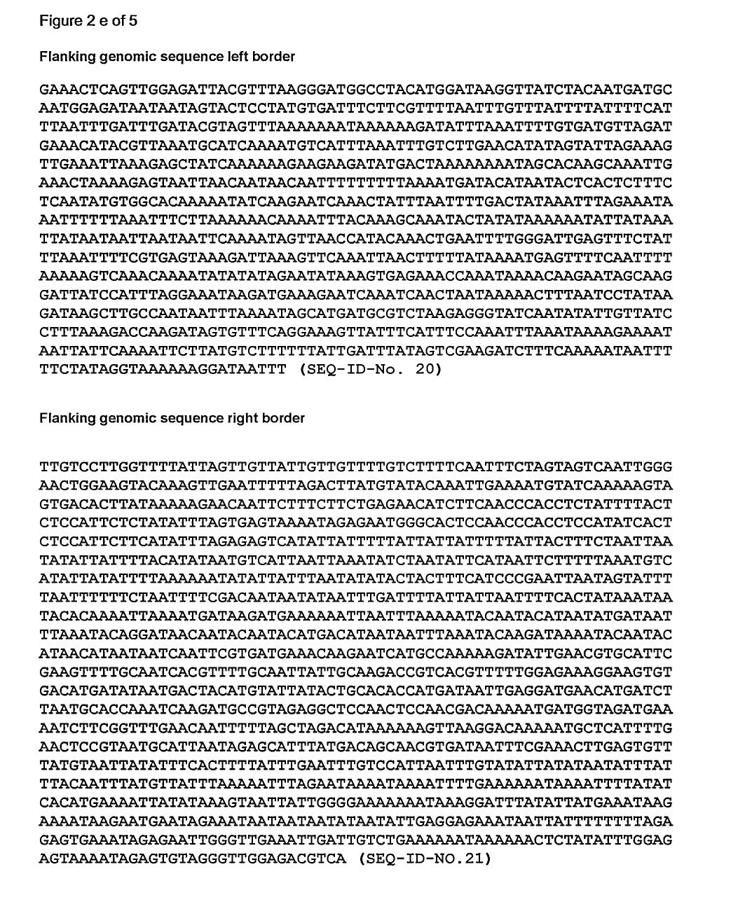

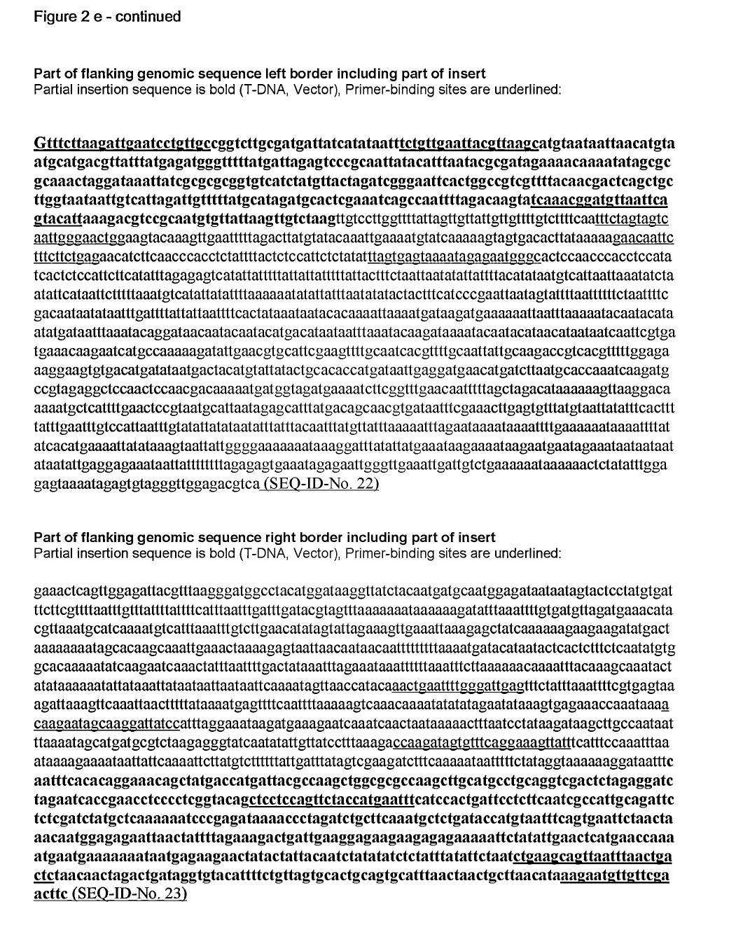

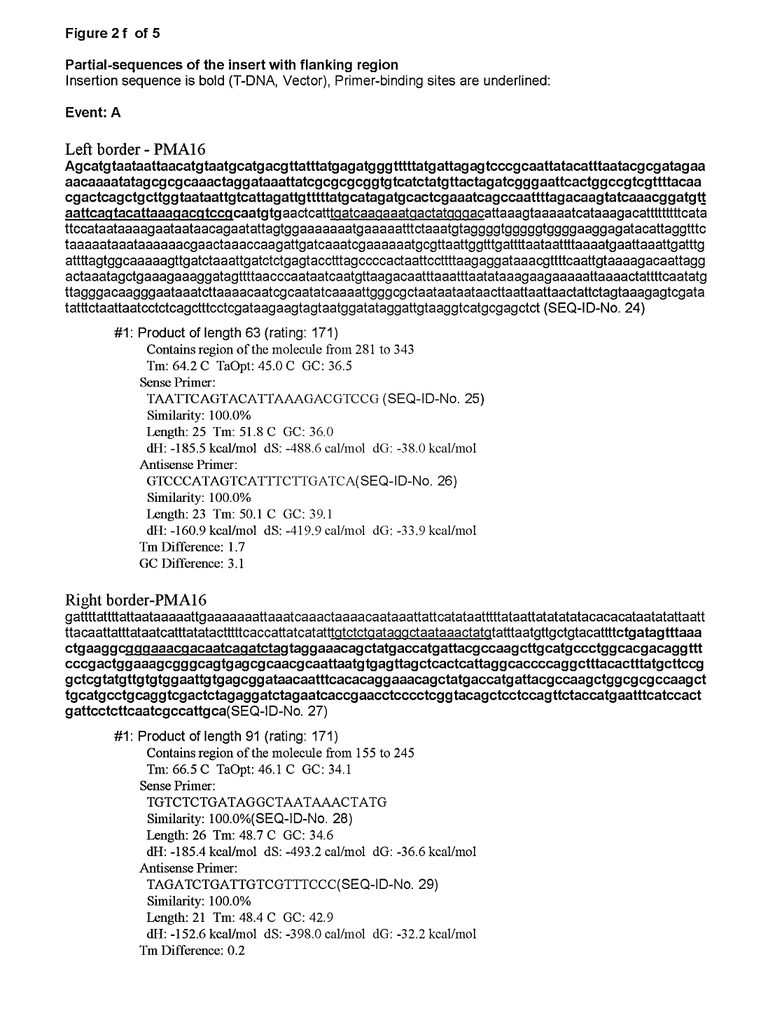

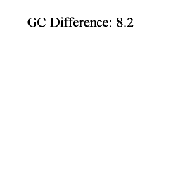

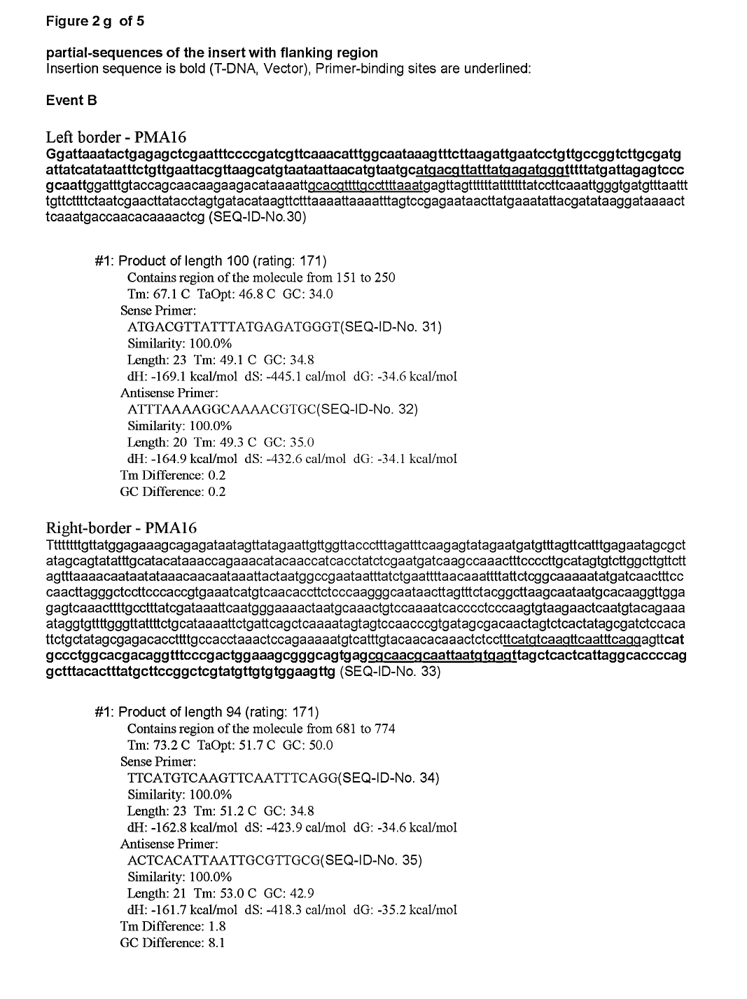

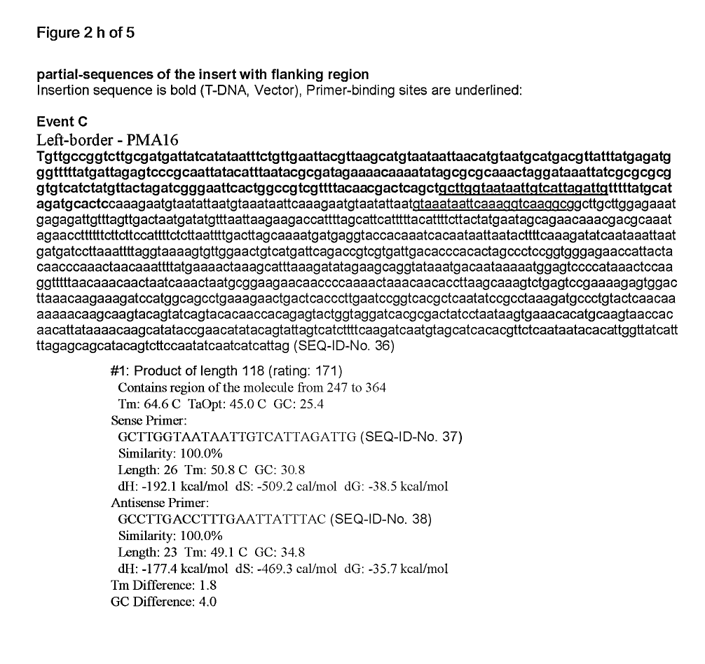

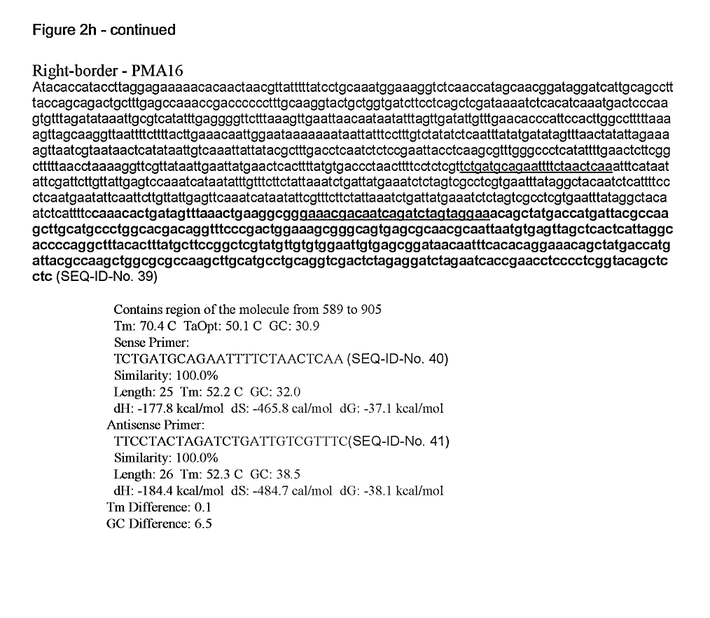

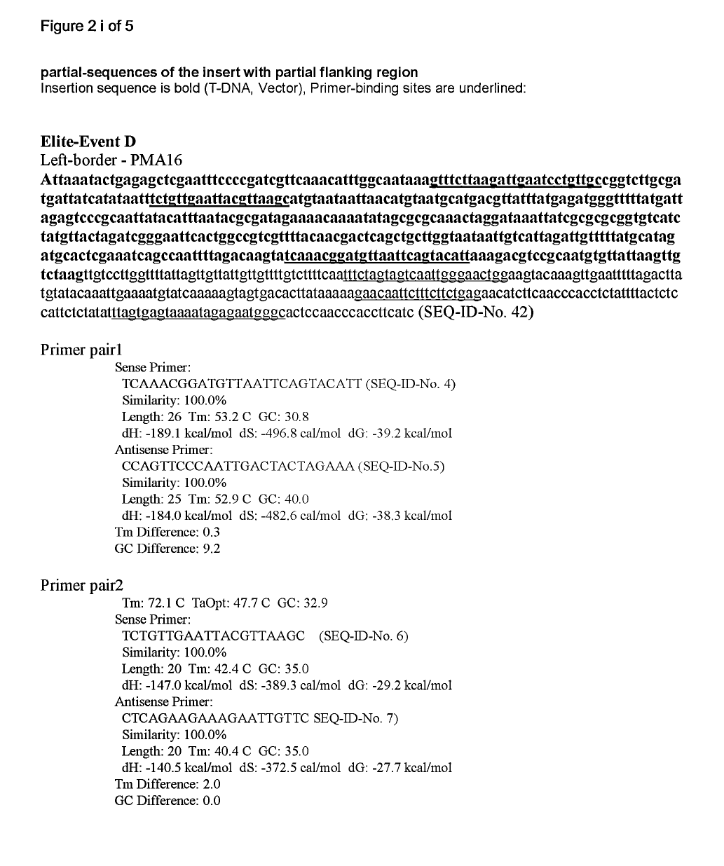

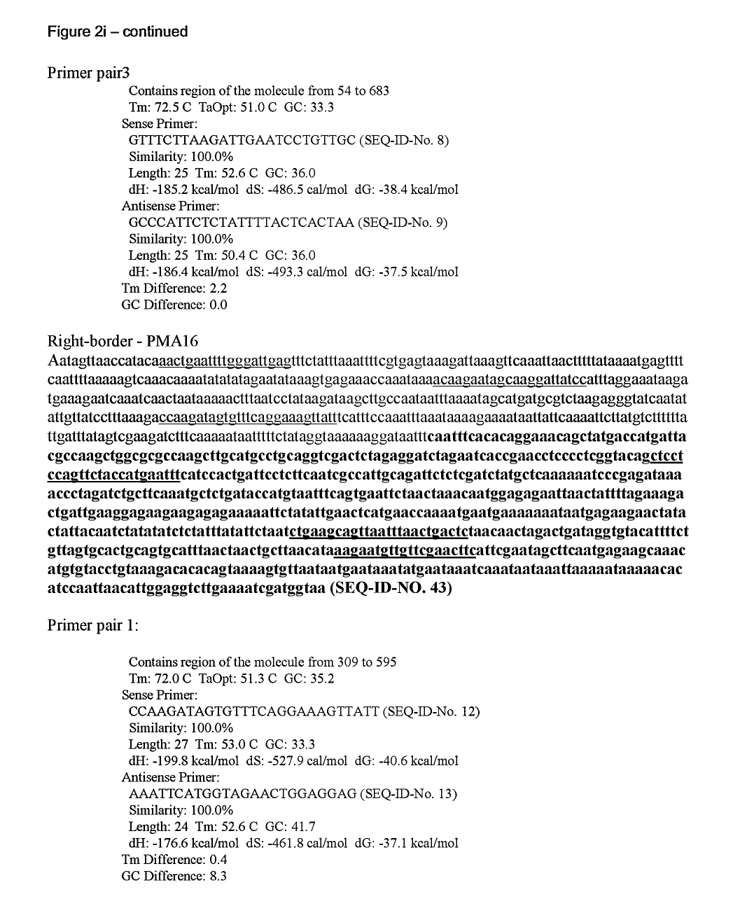

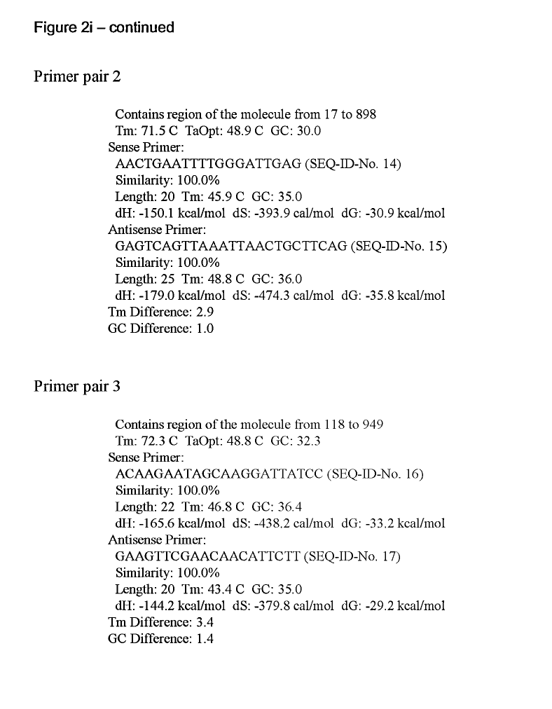

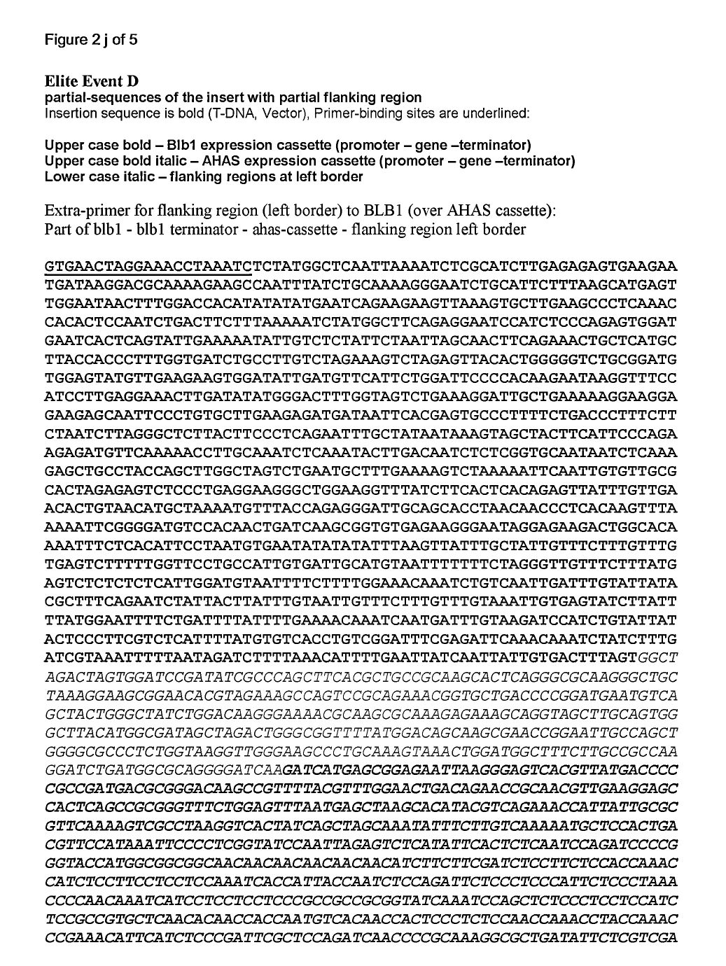

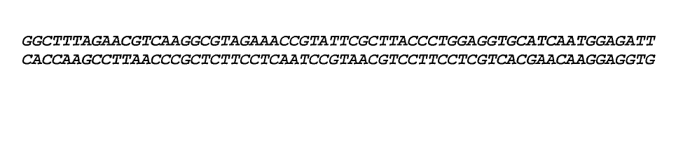

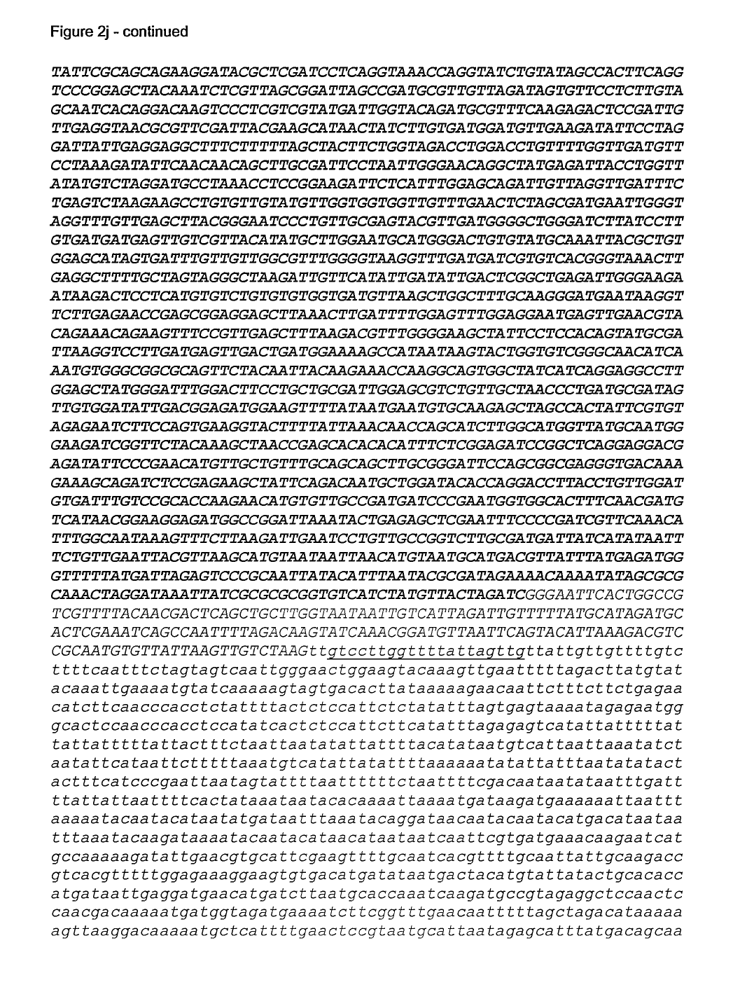

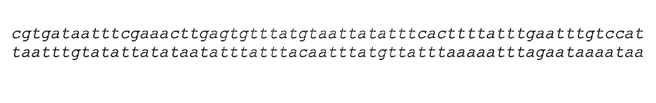

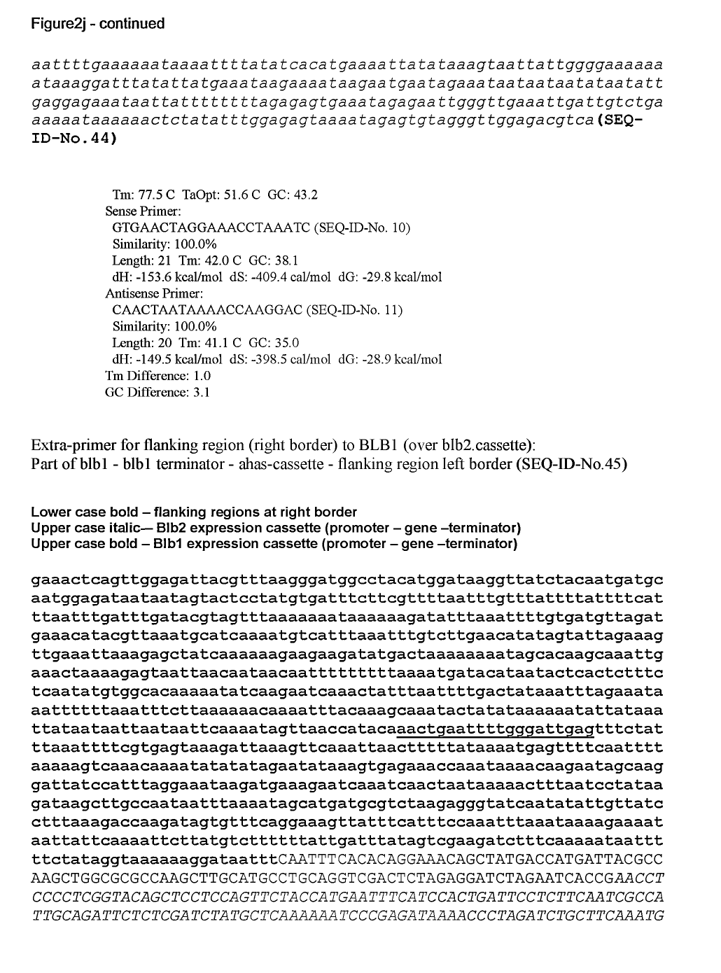

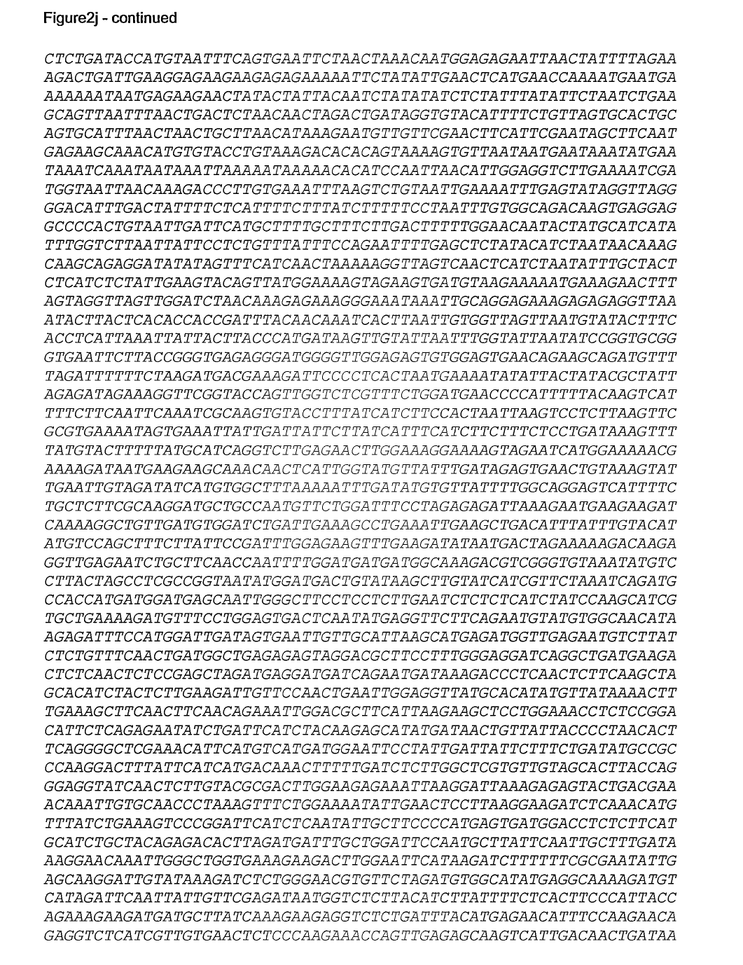

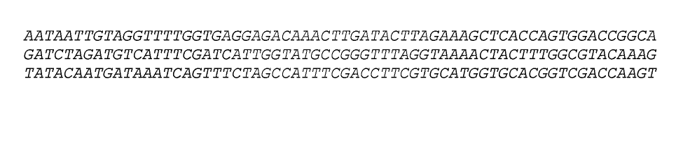

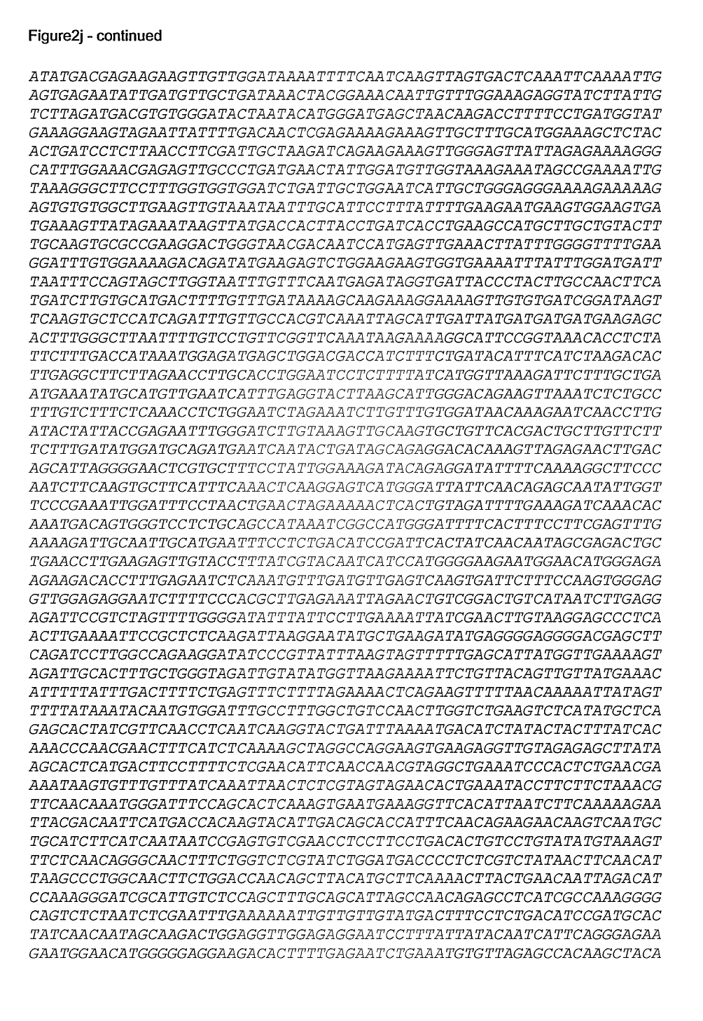

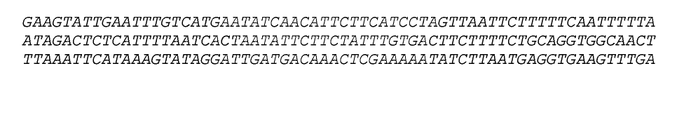

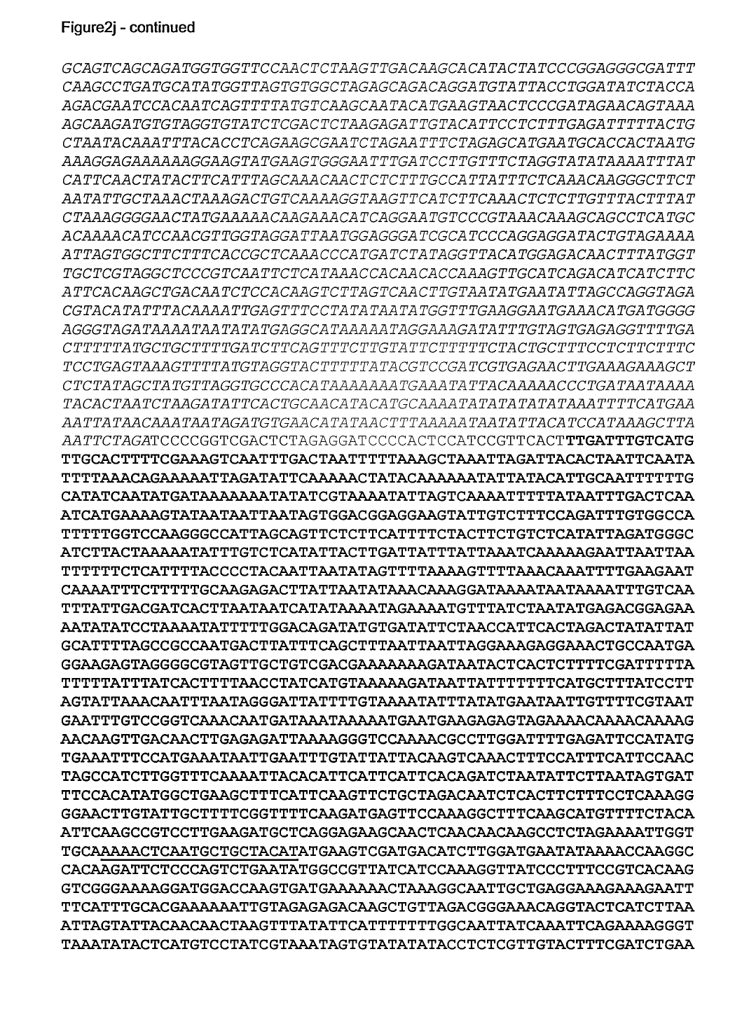

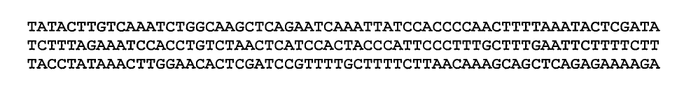

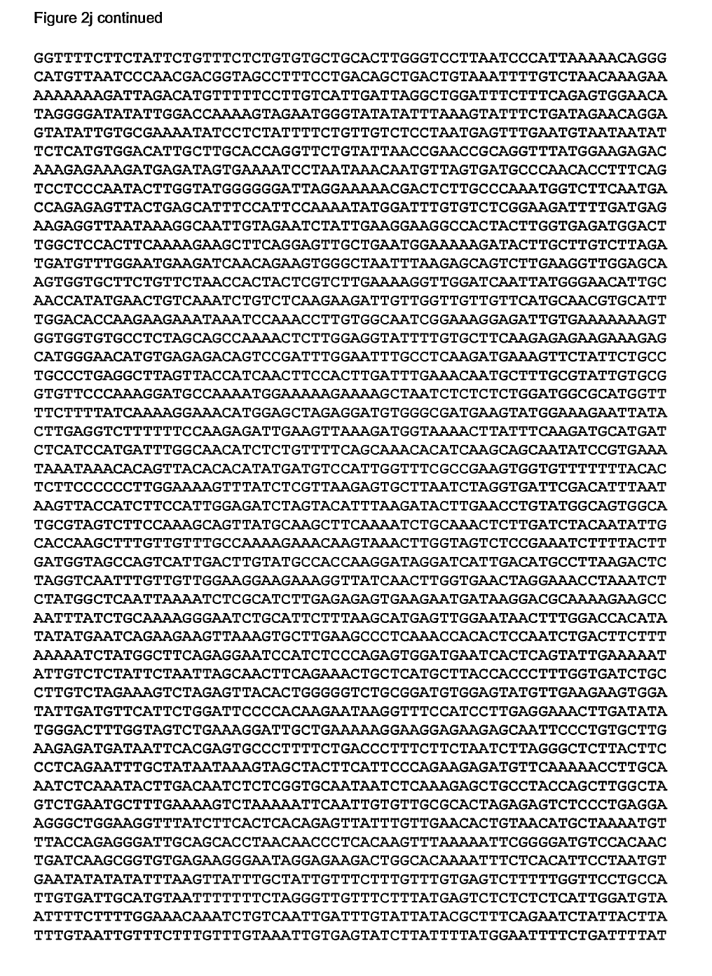

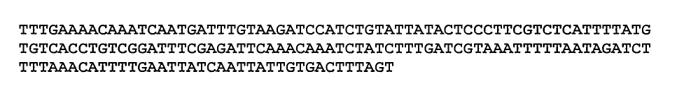

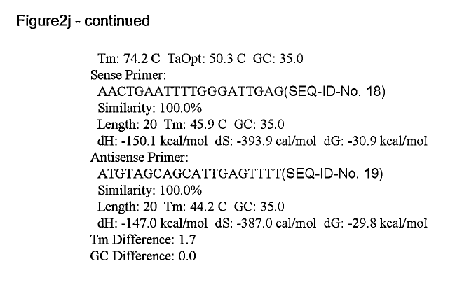

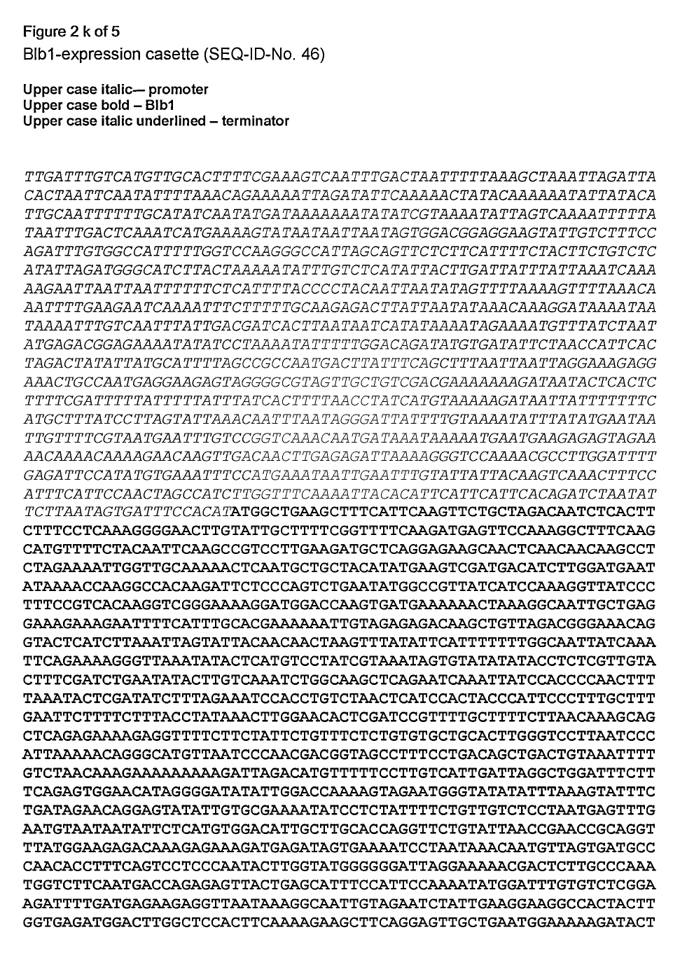

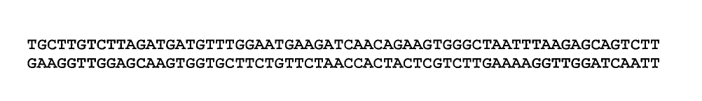

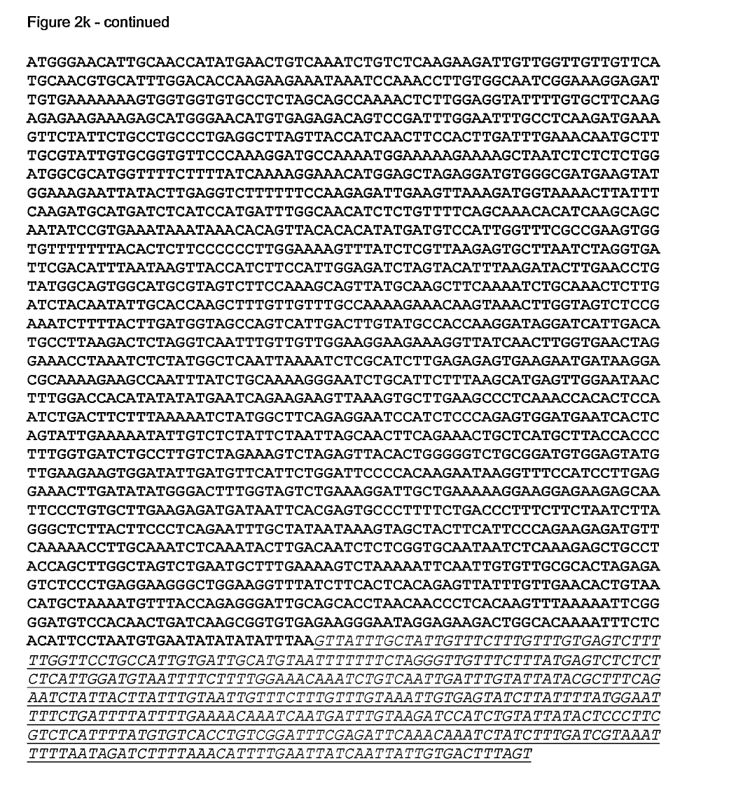

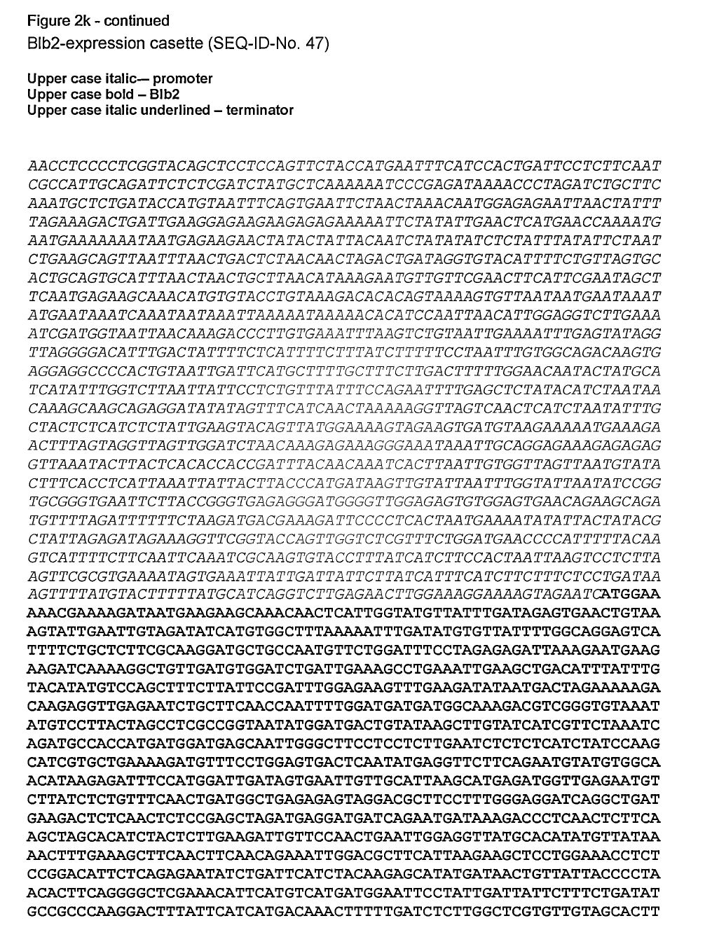

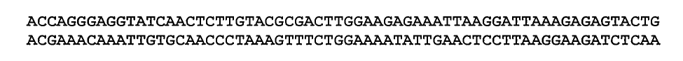

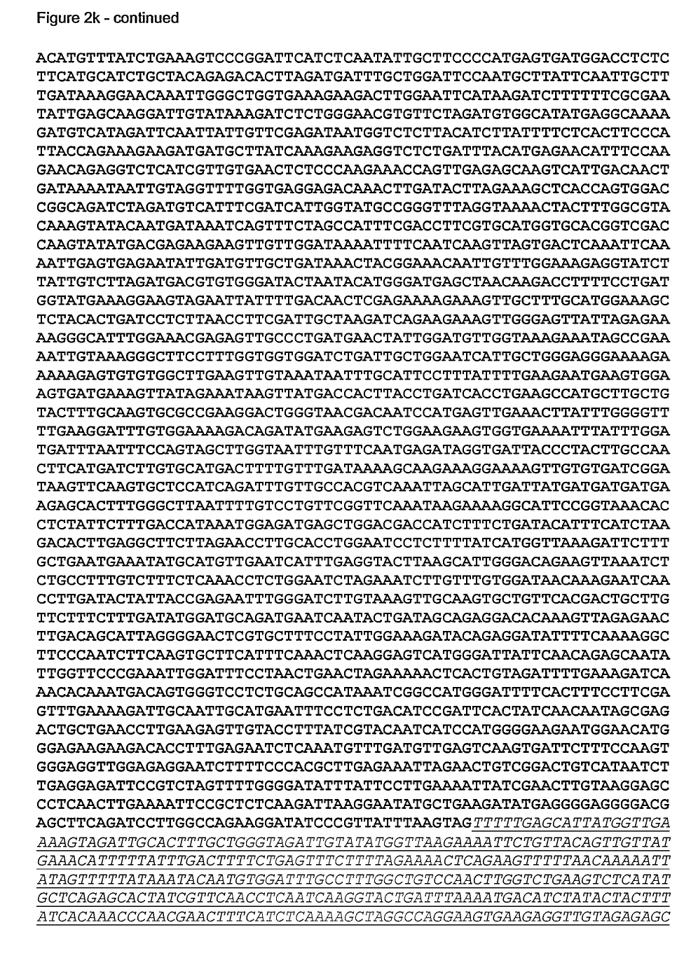

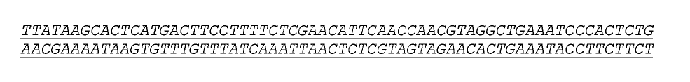

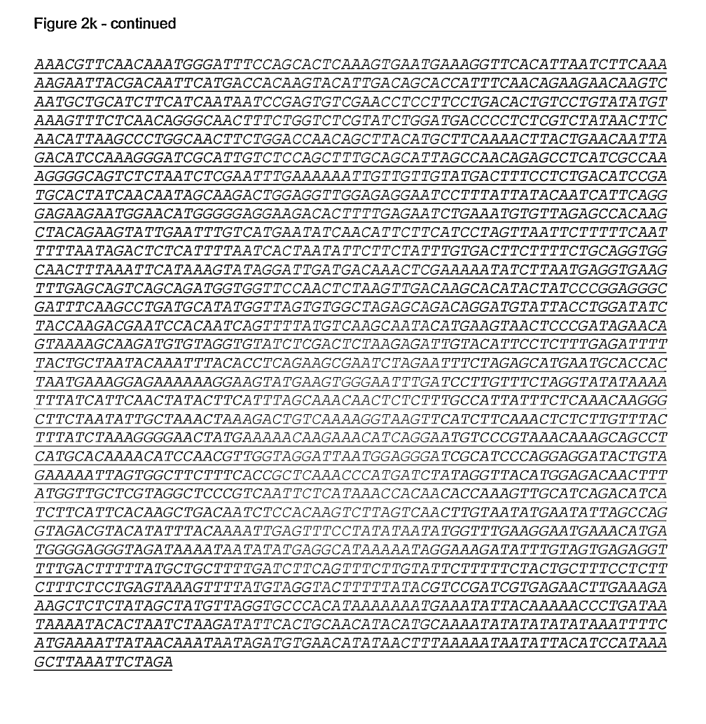

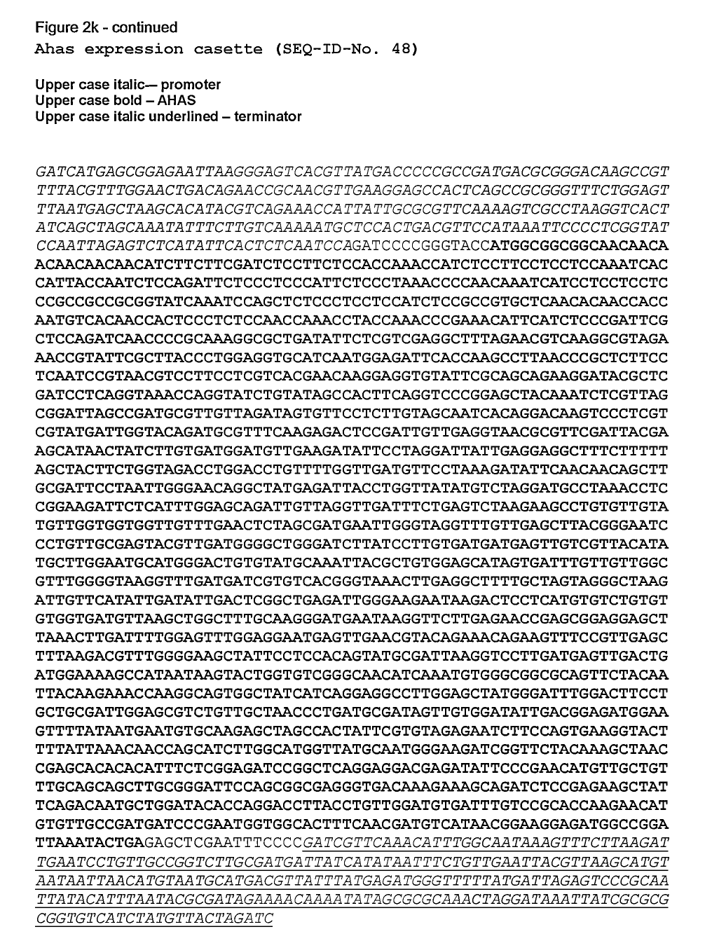

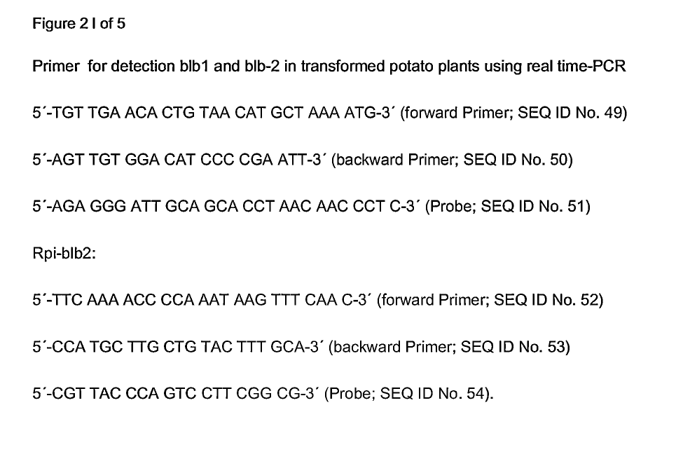

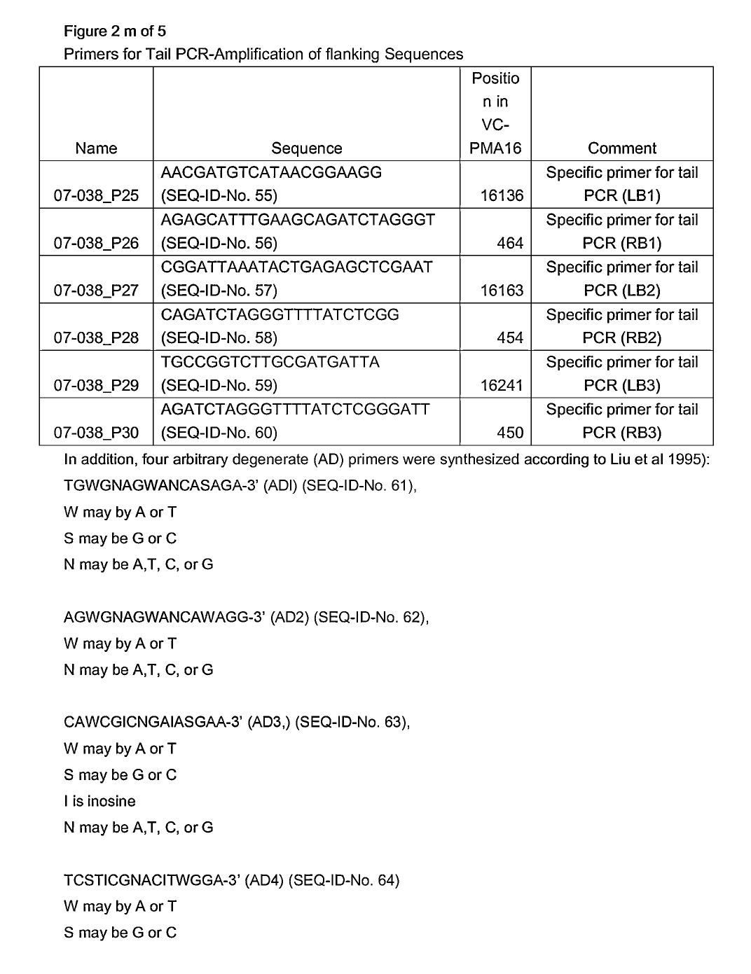

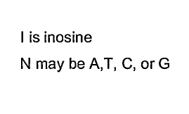

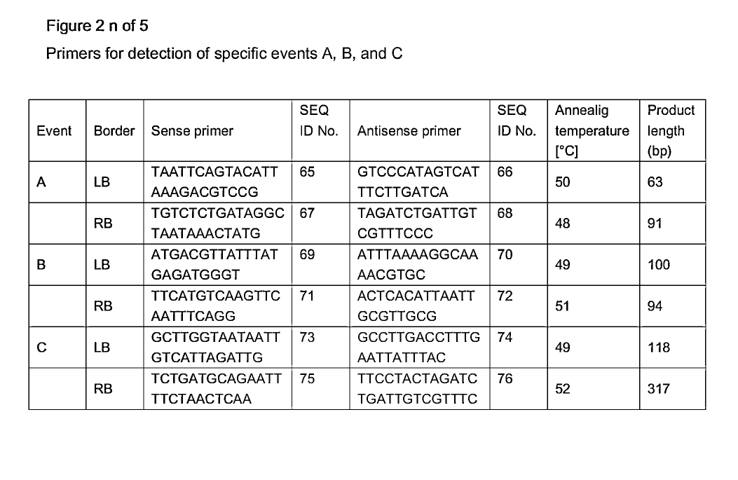

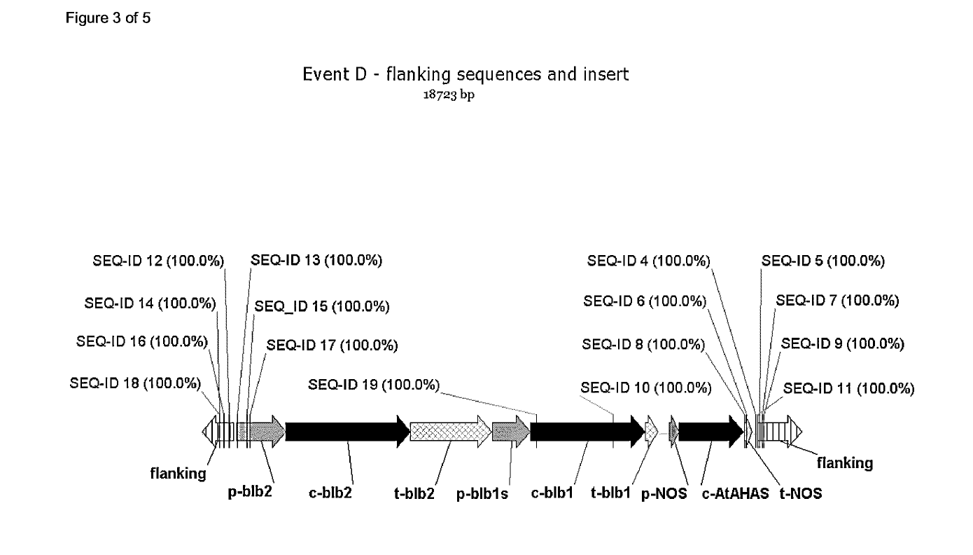

In addition, four arbitrary degenerate (AD) primers were synthesized according to Liu et al 1995): TG(A/T)GNAG(A/T)ANCA(G/C)AGA-3′ (ADI) (SEQ ID NO: 61), AG(A/T)GNAG(A/T)ANCA (A/T)AGG-3′ (AD2) (SEQ ID NO: 62), CA(A/T)CGICNGAIA(G/C)GAA-3′ (AD3, I indicates inosine) (SEQ ID NO: 63), and TC(G/C)TICGNACIT(A/T)GGA-3′ (AD4) (SEQ ID NO: 64). These AD primers have average Tm's of 47-48° C. as calculated with the formula 69.3+0.41 (% GC) −650/L, where L is primer length (cf. FIG. 2l)

Tail PCR was performed basically following Liu et al procedure (Liu et al 1995). Primary TAIL-PCR reactions (20 μl) contained 1× PCR buffer (10 mM Tris-HCl pH 8.3, 50 mM KCI, 1.5 or 2.0 mM MgCl₂, 0.001% gelatin), 200 μM each of dNTPs, 25 ng of genomic DNA, 1 unit of Taq polymerase (Invitrogen), 0.2 μM T-DNA specific primers (07-038_P25 and 07-038_P26) and a given AD primer (2 μM for AD1, 3 μM for AD2 or 4 μM for AD3 and AD4). Primary TAIL-PCR was executed according to the PCR program in Liu et al (1995) in Perkin-Elmer thermal cyclers 9700. Aliquots (1 μl) from 50-fold dilutions of the primary PCR products were applied directly to secondary TAIL-PCR reactions (20 μl) containing 1× PCR buffer, 1 unit of Taq DNA polymerase, 200 μM each of dNTPs, 0.2 μM T-DNA specific primers (07-038_P27 and 07-038_P28) and the same AD primer used in the primary reaction (1.5 μM for AD1, 2.0 μM for AD2 and AD3and AD4). After amplification with 12 super cycles, the secondary TAIL-PCR products (1 μl aliquots of 10 fold dilutions) were re-amplified in 50 μl tertiary reactions with 20 reduced-stringency cycles. Components and their concentrations were the same as in the secondary reaction except that another nested PCR primer was used (07-038_P29 for LB and 07-038_P30 for RB). Amplified products from the reactions were analyzed by agarose gel electrophoresis. Strongly amplified products were recovered and purified with Zymoclean Gel DNA recovery kit (Zymo Research, Calif. 92614, USA). The purified DNA was quantified with Hoechst 33258 dye (Invitrogen) using calf thymus DNA (Invitrogen) as the DNA standard on an FLx800™ Microplate Reader (BioTek Instruments, Winooski, Vt. 05404, USA) according to the fluorometer user manual.

### Example 11

### DNA Sequencing

The purified tertiary PCR products were sequenced with Sanger sequencing using BigDye terminator v3.0 kit according to the manufactuer's protocol (Applied Biosystems, California 92008, USA). The same specific primer used in the tertiary PCR (unlabeled) was used for sequencing.

### Example 12

### Identification of Specific Events by PCR Unless otherwise specified, standard methods as described in Sambrook et al., Molecular

Cloning: A laboratory manual, Cold Spring Harbor 1989, Cold Spring Harbor Laboratory Press are used. Genomic DNA was prepared from the particular potato events by using the DNeasy Plant Mini Kit (Quiagen) for processing of single samples and the DNeasy 96 Plant Kit (Quiagen) for processing of samples in 96 well format. Both kits were used according to the intructions described in the manual. The junction between flanking region and T-DNA insert was amplified from the cDNA by PCR as described in the protocol of the Phusion hot-start, Pfu Ultra, Pfu Turbo or Herculase DNA polymerase (Stratagene, Santa Clara, CA, US).

The composition for the protocol of the Pfu Ultra, Pfu Turbo or Herculase DNA polymerase was as follows: 1× PCR buffer, 0.2 mM of each dNTP, 100 ng cDNA of Arabidopsis thaliana (var Columbia-0) , 50 pmol forward primer, 50 μmol reverse primer, 1 u Pfu Ultra, Pfu Turbo or Herculase DNA polymerase.

The amplification cycles were as follows:

1 cycle of 60 seconds at 98° C., followed by 35 cycles of in each case 10 seconds at 98° C., 30 seconds at specific annealing temperature (see table) and 60 seconds at 72° C., followed by 1 cycle of 10 minutes at 72° C., then 4° C.

The following primer sequences and annealing temperatures were used to specifically amplify event-specific FST-T-DNA junctions:

TABLE 4

| | | | SEQ | | SEQ | Annealig | Product |
|---|---|---|---|---|---|---|---|
| | Flanking | | ID | | ID | temperature | length |
| Event | site | Sense primer | NO: | Antisense primer | NO: | [° C.] | (bp) |
| A | LB | TAATTCAGTACAT | 65 | GTCCCATAGTCA | 66 | 50 | 63 |
| | | TAAAGACGTCCG | | TTTCTTGATCA | | | |
| | RB | TGTCTCTGATAG | 67 | | 68 | 48 | 91 |
| | | GCTAATAAACTAT | | TAGATCTGATTG | | | |
| | | G | | TCGTTTCCC | | | |
| B | LB | ATGACGTTATTTA | 69 | ATTTAAAAGGCA | 70 | 49 | 100 |
| | | TGAGATGGGT | | AAACGTGC | | | |
| | RB | TTCATGTCAAGTT | 71 | ACTCACATTAAT | 72 | 51 | 94 |
| | | CAATTTCAGG | | TGCGTTGCG | | | |
| C | LB | GCTTGGTAATAAT | 73 | GCCTTGACCTTT | 74 | 49 | 118 |
| | | TGTCATTAGATTG | | GAATTATTTAC | | | |
| | RB | TCTGATGCAGAA | 75 | TTCCTACTAGAT | 76 | 52 | 317 |
| | | TTTTCTAACTCAA | | CTGATTGTCGTT | | | |
| | | | | TC | | | |
| D | LB | TCAAACGGATGT | 4 | CCAGTTCCCAAT | 5 | 52 | 131 |
| | | TAATTCAGTACAT | | TGACTACTAGAA | | | |
| | | T | | A | | | |
| | RB | CCAAGATAGTGT | 12 | AAATTCATGGTA | 13 | 52 | 287 |
| | | TTCAGGAAAGTTA | | GAACTGGAGGA | | | |
| | | TT | | G | | | |

The resulting PCR products were analyzed on a 1.5% Agarose-gel. PCR products occur specifically to identify the event. Detailed conditions are given in Table 4.

### Example 13

### Determination of Yield by Field Trials

The various potato events were tested in the field to determine their yield potential. The yield of the events A, B, C and the elite-event D, all showing full Phytophthora resistance, was compared to the non transgenic mother line Fontane and other standard potato varieties, like e.g. Bintje under disease free conditions (full plant protection scheme of transgenic events and control varieties according to good agricultural practice).

Yield trials were performed on more than 15 locations across 3 years (2008-2010). For every experiment a randomized block design with 3-5 block-repetitions was used. Each block was about 10-15 m² in size and planted with 4-6 potato plants per m².

Potatoes were planted in April or May according to local conditions. All plant cultivation management, including plant protection, was performed based on good agricultural practice (GAP). Potato tubers were harvested two weeks after haulm killing in September/October. Harvest was performed either by hand or mechanically. The tuber yield/ha was determined by weighing of the freshly harvested potato tubers at the place of harvest. As control the potato variety Bintje was used. The yield/ha of the standard potato variety Fontane was set to 100% and the relative yield of the non-transgenic line Bintje and the transformed Fontane events A to D was calculated.

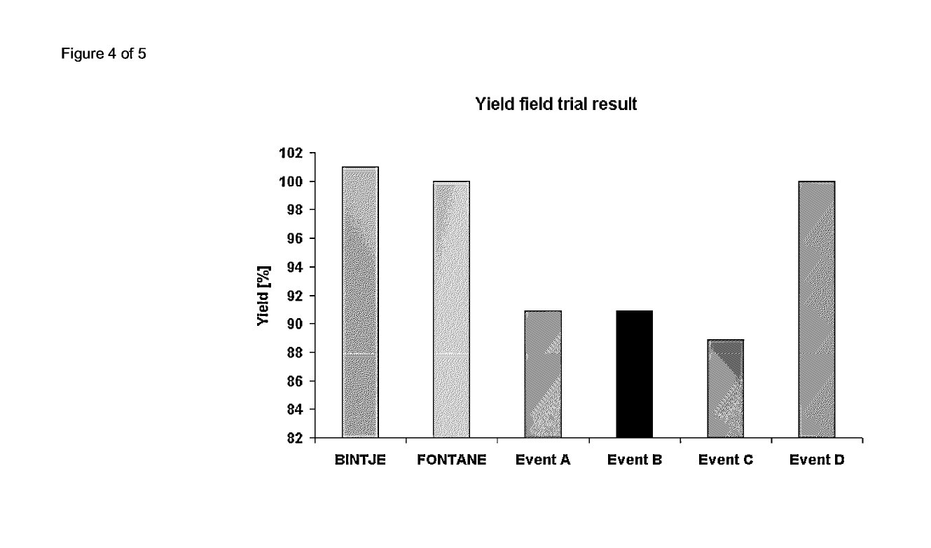

Only the elite-event D showed the same yield/ha compared to the non-transgenic mother line, whereas the other events (e.g. A, B, C) showed a ˜10% yield decrease (cf. FIG. 4).

### Example 14

### Determination of Phytophthora Resistance by Field Trials

The various potato events were tested in the field to determine their resistance phenotype against Phytophthora infestans. All events and the nontransgenic controls (Fontane, as on-transgenic mother line and Bintje as susceptible standard variety) were grown in the field without any fungicide treatments targeting Phytophthora infestans. Resistance trials on more than 20 locations across 5 years (2006-2010) were performed. For every experiment a randomized block design with 3-5 block-repetitions was used. Each block was about 1-15 m² in size and planted with 4-6 potato plants per m².

Potatoes were planted in April or May according to local conditions. All plant cultivation management, excluding plant protection, was performed based on good agricultural practice (GAP). Phytophthora infection occurs naturally.

The rating of disease symptoms was done by trained personal evaluating the diseased leaf area, necrotic lesions, clorotic lesions and potential sporulation of P. infestans.

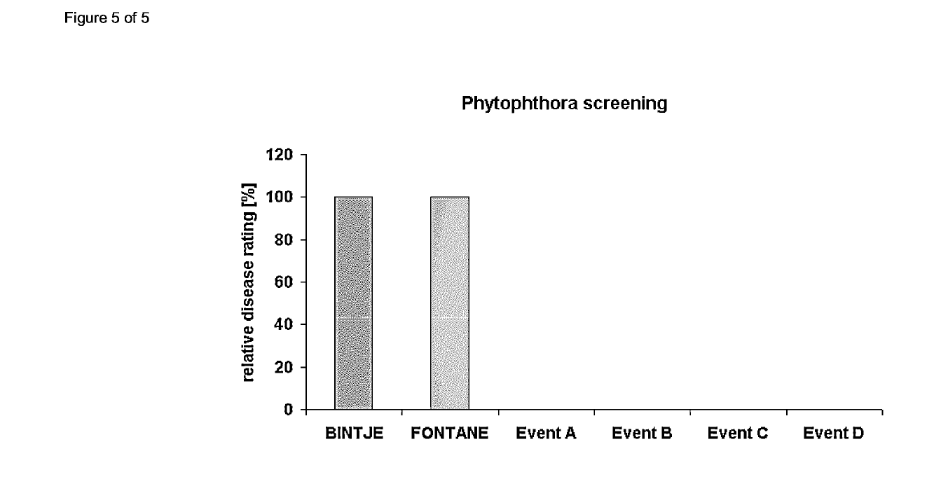

These values were integrated into a disease rating ranging from 0 to 100%. In the scoring system 0% disease means no macroscopically visible symptoms, whereas 100% means that all inoculated leaves are completely brownish and covered with mycelia, so the plant is essentially dead. The mother variety Fontane generally showed a strong infection of all leaves. All leaves are heavily infected and green tissue is rare. In contrast the inoculation of all events A to D led to completely healthy plants with a disease rating of 0%. As susceptible control the standard variety Bintje was used, which is known to be fully susceptible to Phytophthora infection and which showed strong infection (FIG. 5).

1. A Phythophthora-resistant transgenic potato plant, seed, tuber, plant cell or tissue thereof comprising a nucleic acid molecule comprising a nucleotide sequence having at least 80% identity with SEQ ID NO: 1.

2. A Phythophthora-resistant transgenic potato plant, seed, tuber, plant cell or tissue thereof comprising:
a) a recombinant construct comprising a nucleic acid sequence having at least 80% identity with SEQ ID NO: 2 or SEQ ID NO: 3; and
b) further comprising a junction comprising a nucleic acid sequence selected from the group consisting of:
i) a nucleic acid sequence that can be used to amplify a nucleotide fragment of between 126 and 136 basepairs using a polymerase chain reaction with two primers having the nucleotide sequences of SEQ ID NO: 4 and SEQ ID NO: 5;
ii) a nucleic acid sequence that can be used to amplify a nucleotide fragment of between 505 and 515 basepairs using a polymerase chain reaction with two primers having the nucleotide sequences of SEQ ID NO: 6 and SEQ ID NO: 7;
iii) a nucleic acid sequence that can be used to amplify a nucleotide fragment of between 625 and 635 basepairs using a polymerase chain reaction with two primers having the nucleotide sequences of SEQ ID NO: 8 and SEQ ID NO: 9; and/or
iv) a nucleic acid sequence that can be used to amplify a nucleotide fragment of between 4752 and 4762 basepairs using a polymerase chain reaction with two primers having the nucleotide sequences of SEQ ID NO: 10 and SEQ ID NO: 11; and/or
further comprising a junction comprising a nucleic acid sequence selected from the group consisting of:
v) a nucleic acid sequence that can be used to amplify a nucleotide fragment of between 282 and 292 basepairs using a polymerase chain reaction with two primers having the nucleotide sequences of SEQ ID NO: 12 and SEQ ID NO: 13;
vi) a nucleic acid sequence that can be used to amplify a nucleotide fragment of between 877 and 887 basepairs using a polymerase chain reaction with two primers having the nucleotide sequences of SEQ ID NO: 14 and SEQ ID NO: 15;
vii) a nucleic acid sequence that can be used to amplify a nucleotide fragment of between 827 and 837 basepairs using a polymerase chain reaction with two primers having the nucleotide sequences of SEQ ID NO: 16 and SEQ ID NO: 17; and/or
viii) a nucleic acid sequence that can be used to amplify a nucleotide fragment of between 9905 and 9915 basepairs using a polymerase chain reaction with two primers having the nucleotide sequences of SEQ ID NO: 18 and SEQ ID NO: 19.

3. The Phythophthora-resistant transgenic potato, seed, tuber, plant cell or tissue of claim 1 comprising:
a) a recombinant construct comprising a nucleic acid sequence having at least 80% identity with SEQ ID NO: 2 or SEQ ID NO: 3; and
b) further comprising a junction comprising a nucleic acid sequence selected from the group consisting of:
i) a nucleic acid sequence that can be used to amplify a nucleotide fragment of 131 basepairs using a polymerase chain reaction with two primers having the nucleotide sequences of SEQ ID NO: 4 and SEQ ID NO: 5;
ii) a nucleic acid sequence that can be used to amplify a nucleotide fragment 510 basepairs using a polymerase chain reaction with two primers having the nucleotide sequences of SEQ ID NO: 6 and SEQ ID NO: 7;
iii) a nucleic acid sequence that can be used to amplify a nucleotide fragment of 630 basepairs using a polymerase chain reaction with two primers having the nucleotide sequences of SEQ ID NO: 8 and SEQ ID NO: 9; and/or
iv) a nucleic acid sequence that can be used to amplify a nucleotide fragment of 4757 basepairs using a polymerase chain reaction with two primers having the nucleotide sequences of SEQ ID NO: 10 and SEQ ID NO: 11; and/or further comprising a junction comprising a nucleic acid sequence selected from the group consisting of:
v) a nucleic acid sequence that can be used to amplify a nucleotide fragment of 287 basepairs using a polymerase chain reaction with two primers having the nucleotide sequences of SEQ ID NO: 12 and SEQ ID NO: 13;
vi) a nucleic acid sequence that can be used to amplify a nucleotide fragment of 882 basepairs using a polymerase chain reaction with two primers having the nucleotide sequences of SEQ ID NO: 14 and SEQ ID NO: 15;
vii) a nucleic acid sequence that can be used to amplify a nucleotide fragment of 832 basepairs using a polymerase chain reaction with two primers having the nucleotide sequences of SEQ ID NO: 16 and SEQ ID NO: 17; and/or
viii) a nucleic acid sequence that can be used to amplify a nucleotide fragment of 9910 basepairs using a polymerase chain reaction with two primers having the nucleotide sequences of SEQ ID NO: 18 and SEQ ID NO: 19.

4. A method for providing a Phythophthora-resistant transgenic potato plant comprising the following steps:
a) introducing a recombinant nucleic acid comprising a nucleic acid sequence having at least 80% identity with SEQ ID NO: 2 or SEQ ID NO: 3 into the genome of potato plant cells;
b) integrating said recombinant nucleic acid into the genome;
c) regenerating a plant from said plant cells;
d) selecting a plant comprising a nucleic acid comprising a nucleic acid sequence having at least 80% identity with SEQ ID NO: 2 or SEQ ID NO: 3 and a junction comprising a nucleic acid sequence selected from the group consisting of:
i) a nucleic acid sequence that can be used to amplify a nucleotide fragment of between 126 and 136 basepairs using a polymerase chain reaction with two primers having the nucleotide sequences of SEQ ID NO: 4 and SEQ ID NO: 5;
ii) a nucleic acid sequence that can be used to amplify a nucleotide fragment of between 505 and 515 basepairs using a polymerase chain reaction with two primers having the nucleotide sequences of SEQ ID NO: 6 and SEQ ID NO: 7;
iii) a nucleic acid sequence that can be used to amplify a nucleotide fragment of between 625 and 635 basepairs using a polymerase chain reaction with two primers having the nucleotide sequences of SEQ ID NO: 8 and SEQ ID NO: 9; and/or
iv) a nucleic acid sequence that can be used to amplify a nucleotide fragment of between 4752 and 4762 basepairs, using a polymerase chain reaction with two primers having the nucleotide sequences of SEQ ID NO: 10 and SEQ ID NO: 11; and/or
further comprising a junction comprising a nucleic acid sequence selected from the group consisting of:
v) a nucleic acid sequence that can be used to amplify a nucleotide fragment of between 282 and 292 basepairs using a polymerase chain reaction with two primers having the nucleotide sequences of SEQ ID NO: 12 and SEQ ID NO: 13;
vi) a nucleic acid sequence that can be used to amplify a nucleotide fragment of between 877 and 887 basepairs using a polymerase chain reaction with two primers having the nucleotide sequences of SEQ ID NO: 14 and SEQ ID NO: 15;
vii) a nucleic acid sequence that can be used to amplify a nucleotide fragment of between 827 and 837 basepairs using a polymerase chain reaction with two primers having the nucleotide sequences of SEQ ID NO: 16 and SEQ ID NO: 17; and/or
viii) a nucleic acid sequence that can be used to amplify a nucleotide fragment of between 9905 and 9915 basepairs using a polymerase chain reaction with two primers having the nucleotide sequences of SEQ ID NO: 18 and SEQ ID NO: 19.

5. The method for providing a Phythophthora-resistant transgenic potato plant of claim 4, wherein in step d) a plant is selected comprising a nucleic acid comprising a nucleic acid sequence having at least 80% identity with SEQ ID NO: 2 or SEQ ID NO: 3 and a junction comprising a nucleic acid sequence selected from the group consisting of:
i) a nucleic acid sequence that can be used to amplify a nucleotide fragment of 131 basepairs using a polymerase chain reaction with two primers having the nucleotide sequences of SEQ ID NO: 4 and SEQ ID NO: 5;
ii) a nucleic acid sequence that can be used to amplify a nucleotide fragment 510 basepairs, using a polymerase chain reaction with two primers having the nucleotide sequences of SEQ ID NO: 6 and SEQ ID NO: 7;
iii) a nucleic acid sequence that can be used to amplify a nucleotide fragment of 630 basepairs using a polymerase chain reaction with two primers having the nucleotide sequences of SEQ ID NO: 8 and SEQ ID NO: 9; and/or
iv) a nucleic acid sequence that can be used to amplify a nucleotide fragment of 4757 basepairs using a polymerase chain reaction with two primers having the nucleotide sequences of SEQ ID NO: 10 and SEQ ID NO: 11; and/or
further comprising a junction comprising a nucleic sequence selected from the group consisting of:
v) a nucleic acid sequence that can be used to amplify a nucleotide fragment of 287 basepairs using a polymerase chain reaction with two primers having the nucleotide sequences of SEQ ID NO: 12 and SEQ ID NO: 13;
vi) a nucleic acid sequence that can be used to amplify a nucleotide fragment of 882 basepairs using a polymerase chain reaction with two primers having the nucleotide sequences of SEQ ID NO: 14 and SEQ ID NO: 15;
vii) a nucleic acid sequence that can be used to amplify a nucleotide fragment of 832 basepairs using a polymerase chain reaction with two primers having the nucleotide sequences of SEQ ID NO: 16 and SEQ ID NO: 17; and/or
viii) a nucleic acid sequence that can be used to amplify a nucleotide fragment of 9910 basepairs using a polymerase chain reaction with two primers having the nucleotide sequences of SEQ ID NO: 18 and SEQ ID NO: 19.

6. A kit comprising the primer pairs for the detection of the specific integration place, selected from the group consisting of
SEQ ID NO: 4 and 5;
SEQ ID NO: 6 and 7;
SEQ ID NO: 8 and 9;
SEQ ID NO: 10 and 14;
SEQ ID NO: 12 and 13;
SEQ ID NO: 14 and 15;
SEQ ID NO: 16 and and/or
SEQ ID NO: 18 and 19.

7. A detection method for the detection of the specific integration place comprising:
a) isolating a nucleic acid sequence from a potato plant, seed, tuber, plant cell or tissue thereof as a test sample;
b) exposing said test sample, a positive and a negative sample with nucleotide sequence selected from at least one set of primer pairs defined in claim 6 under PCR-conditions; and
c) evaluating the amplification of a nucleotide fragment selected from the group consisting of a nucleotide fragment
i) of between 126 and 136 basepairs when using a polymerase chain reaction with two primers having the nucleotide sequences of SEQ ID NO: 4 and SEQ ID NO: 5;
ii) of between 505 and 515 basepairs when using a polymerase chain reaction with two primers having the nucleotide sequences of SEQ ID NO: 6 and SEQ ID NO: 7, of 625 and 635 basepairs when using a polymerase chain reaction with two primers having the nucleotide sequences of SEQ ID NO: 8 and SEQ ID NO: 9; and/or
iii) of 4752 and 4762 basepairs when using a polymerase chain reaction with two primers having the nucleotide sequences of SEQ ID NO: 10 and SEQ ID NO: 11 and/or selected from the group consisting of a nucleotide fragment;
iv) of between 282 and 292 basepairs when using a polymerase chain reaction with two primers having the nucleotide sequences of SEQ ID NO: 12 and SEQ ID NO: 13;
v) of 877 and 292 basepairs when using a polymerase chain reaction with two primers having the nucleotide sequences of SEQ ID NO: 14 and SEQ ID NO: 15;
vi) of 827 and 837 basepairs when using a polymerase chain reaction with two primers having the nucleotide sequence of SEQ ID NO: 16 and SEQ ID NO: 17; and/or
vii) a nucleic acid sequence that can be used to amplify a nucleotide fragment of 9905 and 9915 basepairs, using a polymerase chain reaction with two primers having the nucleotide sequences of SEQ ID NO: 18 and SEQ ID NO: 19
compared with said positive and negative control.

8. The detection method of claim 7, wherein step c) comprises evaluating the amplification of a nucleotide fragment selected from the group consisting of a nucleotide fragment:
i) of 131 basepairs when using a polymerase chain reaction with two primers having the nucleotide sequences of SEQ ID NO: 4 and SEQ ID NO: 5;
ii) of 510 basepairs when using a polymerase chain reaction with two primers having the nucleotide sequences of SEQ ID NO: 6 and SEQ ID NO: 7;
iii) of 630 basepairs when using a polymerase chain reaction with two primers having the nucleotide sequences of SEQ ID NO: 8 and SEQ ID NO: 9; and/or
iv) of 4757 basepairs when using a polymerase chain reaction with two primers having the nucleotide sequences of SEQ ID NO: 10 and SEQ ID NO: 1 and/or evaluating the amplification of a nucleotide fragment selected from the group consisting of a nucleotide fragment selected from the group consisting of a nucleotide fragment;
v) of 287 basepairs when using a polymerase chain reaction with two primers having the nucleotide sequences of SEQ ID NO: 12 and SEQ ID NO: 13;
vi) of 882 basepairs when using a polymerase chain reaction with two primers having the nucleotide sequences of SEQ ID NO: 14 and SEQ ID NO: 15;
vii) of 832 basepairs when using a polymerase chain reaction with two primers having the nucleotide sequences of SEQ ID NO: 16 and SEQ ID NO: 17; and/or
viii) a nucleic acid sequences that can be used to amplify a nucleotide fragment of 9910 basepairs, using a polymerase chain reaction with two primers having the nucleotide sequence of SEQ ID NO: 18 and SEQ ID NO: 19
compared with said positive and negative control.

9. A plant, seed, tuber, plant cell or tissue thereof detectable by the kit of claim 6.

10. A polynucleotide comprising:
a) a recombinant nucleic acid having at least 80% identity with SEQ ID NO: 2 or SEQ ID NO: 3; and
b) further comprising a junction comprising a nucleic acid sequence selected from the group consisting of:
i) a nucleic acid sequence that can be used to amplify a nucleotide fragment of 131 basepairs using a polymerase chain reaction with two primers having the nucleotide sequences of SEQ ID NO: 4 and SEQ ID NO: 5;
ii) a nucleic acid sequence that can be used to amplify a nucleotide fragment 510 basepairs using a polymerase chain reaction with two primers having the nucleotide sequences of SEQ ID NO: 6 and SEQ ID NO: 7;
iii) a nucleic acid sequence that can be used to amplify a nucleotide fragment of 630 basepairs using a polymerase chain reaction with two primers having the nucleotide sequences of SEQ ID NO: 8 and SEQ ID NO: 9; and/or
iv) a nucleic acid sequence that can be used to amplify a nucleotide fragment of 4757 basepairs using a polymerase chain reaction with two primers having the nucleotide sequences of SEQ ID NO: 10 and SEQ ID NO: 11; and/or
iv) a nucleic acid sequence that can be used to amplify a nucleotide fragment of 287 basepairs using a polymerase chain reaction with two primers having the nucleotide sequences of SEQ ID NO: 12 and SEQ ID NO: 13;
v) a nucleic acid sequence that can be used to amplify a nucleotide fragment of 882 basepairs using a polymerase chain reaction with two primers having the nucleotide sequences of SEQ ID NO: 14 and SEQ ID NO: 15;
vi) a nucleic acid sequence that can be used to amplify a nucleotide fragment of 832 basepairs using a polymerase chain reaction with two primers having the nucleotide sequences of SEQ ID NO: 16 and SEQ ID NO: 17; and/or
vii) a nucleic acid sequence that can be used to amplify a nucleotide fragment of 13234 basepairs using a polymerase chain reaction with two primers having the nucleotide sequences of SEQ ID NO: 18 and SEQ ID NO: 19 stably integrated into a potato plant cell nucleus.

11. A polynucleotide comprising a nucleotide sequence having at least 80% identity SEQ ID NO: 1 stably integrated into a potato plant cell nucleus.

12. The polynucleotide of claim 10, wherein the nucleotide sequence having at least 80% identity SEQ ID NO: 1 comprises the blbl gene and the blb2 gene.

13. The polynucleotide of claim 12, wherein the polynucleotide sequence further comprises one or more of SEQ ID NOS: 4, 5, 6, 7, 8, 9, 10, 11, 12, 13, 14, 15, 16, 17, 18, and/or 19.

14. A Phythophthora-resistant transgenic potato plant, seed, tuber, plant cell or tissue thereof comprising the polynucleotide of claim 10.

15. The Phythophthora-resistant transgenic potato plant, seed, tuber, plant cell or tissue thereof of claim 1, wherein the nucleic acid molecule or recombinant construct comprises the blb1 gene and the blb2 gene.

16. A plant, seed, tuber, plant cell or tissue thereof detectable by the method of claim 7.

17. The Phythophthora-resistant transgenic potato plant, seed, tuber, plant cell or tissue thereof comprising the polynucleotide of claim 11.

18. The Phythophthora-resistant transgenic potato plant, seed, tuber, plant cell or tissue thereof comprising the polynucleotide of claim 12.

19. The Phythophthora-resistant transgenic potato plant, seed, tuber, plant cell or tissue thereof comprising the polynucleotide of claim 13.

20. The Phythophthora-resistant transgenic potato plant, seed, tuber. plant cell or tissue thereof of claim 2, wherein the nucleic acid molecule or recombinant construct comprises the blb1 gene and the blb2 gene.

21. The Phythophthora-resistant transgenic potato plant, seed, tuber, plant cell or tissue thereof of claim 3, wherein the nucleic acid molecule or recombinant construct comprises the blb1 gene and the blb2 gene.


SEQUENCE LISTING (76 sequences)

SEQ ID NO: 1 - DNA, 18723 bp, Artificial sequence
  synthetic construct sequence
gaaactcagttggagattacgtttaagggatggcctacatggataaggttatctacaatgatgcaatggagataataatagtactcctatgtgatttcttcgttttaatttgtttattttattttcatttaatttgatttgatacgtagtttaaaaaaataaaaaagatatttaaattttgtgatgttagatgaaacatacgttaaatgcatcaaaatgtcatttaaatttgtcttgaacatatagtattagaaagttgaaattaaagagctatcaaaaaagaagaagatatgactaaaaaaaatagcacaagcaaattgaaactaaaagagtaattaacaataacaatttttttttaaaatgatacataatactcactctttctcaatatgtggcacaaaaatatcaagaatcaaactatttaattttgactataaatttagaaataaattttttaaatttcttaaaaaacaaaatttacaaagcaaatactatataaaaaatattataaattataataattaataattcaaaatagttaaccatacaaactgaattttgggattgagtttctatttaaattttcgtgagtaaagattaaagttcaaattaactttttataaaatgagttttcaattttaaaaagtcaaacaaaatatatatagaatataaagtgagaaaccaaataaaacaagaatagcaaggattatccatttaggaaataagatgaaagaatcaaatcaactaataaaaactttaatcctataagataagcttgccaataatttaaaatagcatgatgcgtctaagagggtatcaatatattgttatcctttaaagaccaagatagtgtttcaggaaagttatttcatttccaaatttaaataaaagaaaataattattcaaaattcttatgtcttttttattgatttatagtcgaagatctttcaaaaataatttttctataggtaaaaaaggataatttcaatttcacacaggaaacagctatgaccatgattacgccaagctggcgcgccaagcttgcatgcctgcaggtcgactctagaggatctagaatcaccgaacctcccctcggtacagctcctccagttctaccatgaatttcatccactgattcctcttcaatcgccattgcagattctctcgatctatgctcaaaaaatcccgagataaaaccctagatctgcttcaaatgctctgataccatgtaatttcagtgaattctaactaaacaatggagagaattaactattttagaaagactgattgaaggagaagaagagagaaaaattctatattgaactcatgaaccaaaatgaatgaaaaaaataatgagaagaactatactattacaatctatatatctctatttatattctaatctgaagcagttaatttaactgactctaacaactagactgataggtgtacattttctgttagtgcactgcagtgcatttaactaactgcttaacataaagaatgttgttcgaacttcattcgaatagcttcaatgagaagcaaacatgtgtacctgtaaagacacacagtaaaagtgttaataatgaataaatatgaataaatcaaataataaattaaaaataaaaacacatccaattaacattggaggtcttgaaaatcgatggtaattaacaaagacccttgtgaaatttaagtctgtaattgaaaatttgagtataggttaggggacatttgactattttctcattttctttatctttttcctaatttgtggcagacaagtgaggaggccccactgtaattgattcatgcttttgctttcttgactttttggaacaatactatgcatcatatttggtcttaattattcctctgtttatttccagaattttgagctctatacatctaataacaaagcaagcagaggatatatagtttcatcaactaaaaaggttagtcaactcatctaatatttgctactctcatctctattgaagtacagttatggaaaagtagaagtgatgtaagaaaaatgaaagaactttagtaggttagttggatctaacaaagagaaagggaaataaattgcaggagaaagagagaggttaaatacttactcacaccaccgatttacaacaaatcacttaattgtggttagttaatgtatactttcacctcattaaattattacttacccatgataagttgtattaatttggtattaatatccggtgcgggtgaattcttaccgggtgagagggatggggttggagagtgtggagtgaacagaagcagatgttttagattttttctaagatgacgaaagattcccctcactaatgaaaatatattactatacgctattagagatagaaaggttcggtaccagttggtctcgtttctggatgaaccccatttttacaagtcattttcttcaattcaaatcgcaagtgtacctttatcatcttccactaattaagtcctcttaagttcgcgtgaaaatagtgaaattattgattattcttatcatttcatcttctttctcctgataaagttttatgtactttttatgcatcaggtcttgagaacttggaaaggaaaagtagaatcatggaaaaacgaaaagataatgaagaagcaaacaactcattggtatgttatttgatagagtgaactgtaaagtattgaattgtagatatcatgtggctttaaaaatttgatatgtgttattttggcaggagtcattttctgctcttcgcaaggatgctgccaatgttctggatttcctagagagattaaagaatgaagaagatcaaaaggctgttgatgtggatctgattgaaagcctgaaattgaagctgacatttatttgtacatatgtccagctttcttattccgatttggagaagtttgaagatataatgactagaaaaagacaagaggttgagaatctgcttcaaccaattttggatgatgatggcaaagacgtcgggtgtaaatatgtccttactagcctcgccggtaatatggatgactgtataagcttgtatcatcgttctaaatcagatgccaccatgatggatgagcaattgggcttcctcctcttgaatctctctcatctatccaagcatcgtgctgaaaagatgtttcctggagtgactcaatatgaggttcttcagaatgtatgtggcaacataagagatttccatggattgatagtgaattgttgcattaagcatgagatggttgagaatgtcttatctctgtttcaactgatggctgagagagtaggacgcttcctttgggaggatcaggctgatgaagactctcaactctccgagctagatgaggatgatcagaatgataaagaccctcaactcttcaagctagcacatctactcttgaagattgttccaactgaattggaggttatgcacatatgttataaaactttgaaagcttcaacttcaacagaaattggacgcttcattaagaagctcctggaaacctctccggacattctcagagaatatctgattcatctacaagagcatatgataactgttattacccctaacacttcaggggctcgaaacattcatgtcatgatggaattcctattgattattctttctgatatgccgcccaaggactttattcatcatgacaaactttttgatctcttggctcgtgttgtagcacttaccagggaggtatcaactcttgtacgcgacttggaagagaaattaaggattaaagagagtactgacgaaacaaattgtgcaaccctaaagtttctggaaaatattgaactccttaaggaagatctcaaacatgtttatctgaaagtcccggattcatctcaatattgcttccccatgagtgatggacctctcttcatgcatctgctacagagacacttagatgatttgctggattccaatgcttattcaattgctttgataaaggaacaaattgggctggtgaaagaagacttggaattcataagatcttttttcgcgaatattgagcaaggattgtataaagatctctgggaacgtgttctagatgtggcatatgaggcaaaagatgtcatagattcaattattgttcgagataatggtctcttacatcttattttctcacttcccattaccagaaagaagatgatgcttatcaaagaagaggtctctgatttacatgagaacatttccaagaacagaggtctcatcgttgtgaactctcccaagaaaccagttgagagcaagtcattgacaactgataaaataattgtaggttttggtgaggagacaaacttgatacttagaaagctcaccagtggaccggcagatctagatgtcatttcgatcattggtatgccgggtttaggtaaaactactttggcgtacaaagtatacaatgataaatcagtttctagccatttcgaccttcgtgcatggtgcacggtcgaccaagtatatgacgagaagaagttgttggataaaattttcaatcaagttagtgactcaaattcaaaattgagtgagaatattgatgttgctgataaactacggaaacaattgtttggaaagaggtatcttattgtcttagatgacgtgtgggatactaatacatgggatgagctaacaagaccttttcctgatggtatgaaaggaagtagaattattttgacaactcgagaaaagaaagttgctttgcatggaaagctctacactgatcctcttaaccttcgattgctaagatcagaagaaagttgggagttattagagaaaagggcatttggaaacgagagttgccctgatgaactattggatgttggtaaagaaatagccgaaaattgtaaagggcttcctttggtggtggatctgattgctggaatcattgctgggagggaaaagaaaaagagtgtgtggcttgaagttgtaaataatttgcattcctttattttgaagaatgaagtggaagtgatgaaagttatagaaataagttatgaccacttacctgatcacctgaagccatgcttgctgtactttgcaagtgcgccgaaggactgggtaacgacaatccatgagttgaaacttatttggggttttgaaggatttgtggaaaagacagatatgaagagtctggaagaagtggtgaaaatttatttggatgatttaatttccagtagcttggtaatttgtttcaatgagataggtgattaccctacttgccaacttcatgatcttgtgcatgacttttgtttgataaaagcaagaaaggaaaagttgtgtgatcggataagttcaagtgctccatcagatttgttgccacgtcaaattagcattgattatgatgatgatgaagagcactttgggcttaattttgtcctgttcggttcaaataagaaaaggcattccggtaaacacctctattctttgaccataaatggagatgagctggacgaccatctttctgatacatttcatctaagacacttgaggcttcttagaaccttgcacctggaatcctcttttatcatggttaaagattctttgctgaatgaaatatgcatgttgaatcatttgaggtacttaagcattgggacagaagttaaatctctgcctttgtctttctcaaacctctggaatctagaaatcttgtttgtggataacaaagaatcaaccttgatactattaccgagaatttgggatcttgtaaagttgcaagtgctgttcacgactgcttgttctttctttgatatggatgcagatgaatcaatactgatagcagaggacacaaagttagagaacttgacagcattaggggaactcgtgctttcctattggaaagatacagaggatattttcaaaaggcttcccaatcttcaagtgcttcatttcaaactcaaggagtcatgggattattcaacagagcaatattggttcccgaaattggatttcctaactgaactagaaaaactcactgtagattttgaaagatcaaacacaaatgacagtgggtcctctgcagccataaatcggccatgggattttcactttccttcgagtttgaaaagattgcaattgcatgaatttcctctgacatccgattcactatcaacaatagcgagactgctgaaccttgaagagttgtacctttatcgtacaatcatccatggggaagaatggaacatgggagaagaagacacctttgagaatctcaaatgtttgatgttgagtcaagtgattctttccaagtgggaggttggagaggaatcttttcccacgcttgagaaattagaactgtcggactgtcataatcttgaggagattccgtctagttttggggatatttattccttgaaaattatcgaacttgtaaggagccctcaacttgaaaattccgctctcaagattaaggaatatgctgaagatatgaggggaggggacgagcttcagatccttggccagaaggatatcccgttatttaagtagtttttgagcattatggttgaaaagtagattgcactttgctgggtagattgtatatggttaagaaaattctgttacagttgttatgaaacatttttatttgacttttctgagtttcttttagaaaactcagaagtttttaacaaaaattatagtttttataaatacaatgtggatttgcctttggctgtccaacttggtctgaagtctcatatgctcagagcactatcgttcaacctcaatcaaggtactgatttaaaatgacatctatactactttatcacaaacccaacgaactttcatctcaaaagctaggccaggaagtgaagaggttgtagagagcttataagcactcatgacttccttttctcgaacattcaaccaacgtaggctgaaatcccactctgaacgaaaataagtgtttgtttatcaaattaactctcgtagtagaacactgaaataccttcttctaaacgttcaacaaatgggatttccagcactcaaagtgaatgaaaggttcacattaatcttcaaaaagaattacgacaattcatgaccacaagtacattgacagcaccatttcaacagaagaacaagtcaatgctgcatcttcatcaataatccgagtgtcgaacctccttcctgacactgtcctgtatatgtaaagtttctcaacagggcaactttctggtctcgtatctggatgacccctctcgtctataacttcaacattaagccctggcaacttctggaccaacagcttacatgcttcaaaacttactgaacaattagacatccaaagggatcgcattgtctccagctttgcagcattagccaacagagcctcatcgccaaaggggcagtctctaatctcgaatttgaaaaaattgttgttgtatgactttcctctgacatccgatgcactatcaacaatagcaagactggaggttggagaggaatcctttattatacaatcattcagggagaagaatggaacatgggggaggaagacacttttgagaatctgaaatgtgttagagccacaagctacagaagtattgaatttgtcatgaatatcaacattcttcatcctagttaattctttttcaatttttaatagactctcattttaatcactaatattcttctatttgtgacttcttttctgcaggtggcaactttaaattcataaagtataggattgatgacaaactcgaaaaatatcttaatgaggtgaagtttgagcagtcagcagatggtggttccaactctaagttgacaagcacatactatcccggagggcgatttcaagcctgatgcatatggttagtgtggctagagcagacaggatgtattacctggatatctaccaagacgaatccacaatcagttttatgtcaagcaatacatgaagtaactcccgatagaacagtaaaagcaagatgtgtaggtgtatctcgactctaagagattgtacattcctctttgagatttttactgctaatacaaatttacacctcagaagcgaatctagaatttctagagcatgaatgcaccactaatgaaaggagaaaaaaggaagtatgaagtgggaatttgatccttgtttctaggtatataaaatttatcattcaactatacttcatttagcaaacaactctctttgccattatttctcaaacaagggcttctaatattgctaaactaaagactgtcaaaaggtaagttcatcttcaaactctcttgtttactttatctaaaggggaactatgaaaaacaagaaacatcaggaatgtcccgtaaacaaagcagcctcatgcacaaaacatccaacgttggtaggattaatggagggatcgcatcccaggaggatactgtagaaaaattagtggcttctttcaccgctcaaacccatgatctataggttacatggagacaactttatggttgctcgtaggctcccgtcaattctcataaaccacaacaccaaagttgcatcagacatcatcttcattcacaagctgacaatctccacaagtcttagtcaacttgtaatatgaatattagccaggtagacgtacatatttacaaaattgagtttcctatataatatggtttgaaggaatgaaacatgatggggagggtagataaaataatatatgaggcataaaaataggaaagatatttgtagtgagaggttttgactttttatgctgcttttgatcttcagtttcttgtattctttttctactgctttcctcttctttctcctgagtaaagttttatgtaggtactttttatacgtccgatcgtgagaacttgaaagaaagctctctatagctatgttaggtgcccacataaaaaaatgaaatattacaaaaaccctgataataaaatacactaatctaagatattcactgcaacatacatgcaaaatatatatatataaattttcatgaaaattataacaaataatagatgtgaacatataactttaaaaataatattacatccataaagcttaaattctagatccccggtcgactctagaggatccccactccatccgttcactttgatttgtcatgttgcacttttcgaaagtcaatttgactaatttttaaagctaaattagattacactaattcaatattttaaacagaaaaattagatattcaaaaactatacaaaaaatattatacattgcaattttttgcatatcaatatgataaaaaaatatatcgtaaaatattagtcaaaatttttataatttgactcaaatcatgaaaagtataataattaatagtggacggaggaagtattgtctttccagatttgtggccatttttggtccaagggccattagcagttctcttcattttctacttctgtctcatattagatgggcatcttactaaaaatatttgtctcatattacttgattatttattaaatcaaaaagaattaattaattttttctcattttacccctacaattaatatagttttaaaagttttaaacaaattttgaagaatcaaaatttctttttgcaagagacttattaatataaacaaaggataaaataataaaatttgtcaatttattgacgatcacttaataatcatataaaatagaaaatgtttatctaatatgagacggagaaaatatatcctaaaatatttttggacagatatgtgatattctaaccattcactagactatattatgcattttagccgccaatgacttatttcagctttaattaattaggaaagaggaaactgccaatgaggaagagtaggggcgtagttgctgtcgacgaaaaaaagataatactcactcttttcgatttttatttttatttatcacttttaacctatcatgtaaaaagataattatttttttcatgctttatccttagtattaaacaatttaatagggattattttgtaaaatatttatatgaataattgttttcgtaatgaatttgtccggtcaaacaatgataaataaaaatgaatgaagagagtagaaaacaaaacaaaagaacaagttgacaacttgagagattaaaagggtccaaaacgccttggattttgagattccatatgtgaaatttccatgaaataattgaatttgtattattacaagtcaaactttccatttcattccaactagccatcttggtttcaaaattacacattcattcattcacagatctaatattcttaatagtgatttccacatatggctgaagctttcattcaagttctgctagacaatctcacttctttcctcaaaggggaacttgtattgcttttcggttttcaagatgagttccaaaggctttcaagcatgttttctacaattcaagccgtccttgaagatgctcaggagaagcaactcaacaacaagcctctagaaaattggttgcaaaaactcaatgctgctacatatgaagtcgatgacatcttggatgaatataaaaccaaggccacaagattctcccagtctgaatatggccgttatcatccaaaggttatccctttccgtcacaaggtcgggaaaaggatggaccaagtgatgaaaaaactaaaggcaattgctgaggaaagaaagaattttcatttgcacgaaaaaattgtagagagacaagctgttagacgggaaacaggtactcatcttaaattagtattacaacaactaagtttatattcatttttttggcaattatcaaattcagaaaagggttaaatatactcatgtcctatcgtaaatagtgtatatatacctctcgttgtactttcgatctgaatatacttgtcaaatctggcaagctcagaatcaaattatccaccccaacttttaaatactcgatatctttagaaatccacctgtctaactcatccactacccattccctttgctttgaattcttttctttacctataaacttggaacactcgatccgttttgcttttcttaacaaagcagctcagagaaaagaggttttcttctattctgtttctctgtgtgctgcacttgggtccttaatcccattaaaaacagggcatgttaatcccaacgacggtagcctttcctgacagctgactgtaaattttgtctaacaaagaaaaaaaaagattagacatgtttttccttgtcattgattaggctggatttctttcagagtggaacataggggatatattggaccaaaagtagaatgggtatatatttaaagtatttctgatagaacaggagtatattgtgcgaaaatatcctctattttctgttgtctcctaatgagtttgaatgtaataatattctcatgtggacattgcttgcaccaggttctgtattaaccgaaccgcaggtttatggaagagacaaagagaaagatgagatagtgaaaatcctaataaacaatgttagtgatgcccaacacctttcagtcctcccaatacttggtatggggggattaggaaaaacgactcttgcccaaatggtcttcaatgaccagagagttactgagcatttccattccaaaatatggatttgtgtctcggaagattttgatgagaagaggttaataaaggcaattgtagaatctattgaaggaaggccactacttggtgagatggacttggctccacttcaaaagaagcttcaggagttgctgaatggaaaaagatacttgcttgtcttagatgatgtttggaatgaagatcaacagaagtgggctaatttaagagcagtcttgaaggttggagcaagtggtgcttctgttctaaccactactcgtcttgaaaaggttggatcaattatgggaacattgcaaccatatgaactgtcaaatctgtctcaagaagattgttggttgttgttcatgcaacgtgcatttggacaccaagaagaaataaatccaaaccttgtggcaatcggaaaggagattgtgaaaaaaagtggtggtgtgcctctagcagccaaaactcttggaggtattttgtgcttcaagagagaagaaagagcatgggaacatgtgagagacagtccgatttggaatttgcctcaagatgaaagttctattctgcctgccctgaggcttagttaccatcaacttccacttgatttgaaacaatgctttgcgtattgtgcggtgttcccaaaggatgccaaaatggaaaaagaaaagctaatctctctctggatggcgcatggttttcttttatcaaaaggaaacatggagctagaggatgtgggcgatgaagtatggaaagaattatacttgaggtcttttttccaagagattgaagttaaagatggtaaaacttatttcaagatgcatgatctcatccatgatttggcaacatctctgttttcagcaaacacatcaagcagcaatatccgtgaaataaataaacacagttacacacatatgatgtccattggtttcgccgaagtggtgtttttttacactcttccccccttggaaaagtttatctcgttaagagtgcttaatctaggtgattcgacatttaataagttaccatcttccattggagatctagtacatttaagatacttgaacctgtatggcagtggcatgcgtagtcttccaaagcagttatgcaagcttcaaaatctgcaaactcttgatctacaatattgcaccaagctttgttgtttgccaaaagaaacaagtaaacttggtagtctccgaaatcttttacttgatggtagccagtcattgacttgtatgccaccaaggataggatcattgacatgccttaagactctaggtcaatttgttgttggaaggaagaaaggttatcaacttggtgaactaggaaacctaaatctctatggctcaattaaaatctcgcatcttgagagagtgaagaatgataaggacgcaaaagaagccaatttatctgcaaaagggaatctgcattctttaagcatgagttggaataactttggaccacatatatatgaatcagaagaagttaaagtgcttgaagccctcaaaccacactccaatctgacttctttaaaaatctatggcttcagaggaatccatctcccagagtggatgaatcactcagtattgaaaaatattgtctctattctaattagcaacttcagaaactgctcatgcttaccaccctttggtgatctgccttgtctagaaagtctagagttacactgggggtctgcggatgtggagtatgttgaagaagtggatattgatgttcattctggattccccacaagaataaggtttccatccttgaggaaacttgatatatgggactttggtagtctgaaaggattgctgaaaaaggaaggagaagagcaattccctgtgcttgaagagatgataattcacgagtgcccttttctgaccctttcttctaatcttagggctcttacttccctcagaatttgctataataaagtagctacttcattcccagaagagatgttcaaaaaccttgcaaatctcaaatacttgacaatctctcggtgcaataatctcaaagagctgcctaccagcttggctagtctgaatgctttgaaaagtctaaaaattcaattgtgttgcgcactagagagtctccctgaggaagggctggaaggtttatcttcactcacagagttatttgttgaacactgtaacatgctaaaatgtttaccagagggattgcagcacctaacaaccctcacaagtttaaaaattcggggatgtccacaactgatcaagcggtgtgagaagggaataggagaagactggcacaaaatttctcacattcctaatgtgaatatatatatttaagttatttgctattgtttctttgtttgtgagtctttttggttcctgccattgtgattgcatgtaatttttttctagggttgtttctttatgagtctctctctcattggatgtaattttcttttggaaacaaatctgtcaattgatttgtattatacgctttcagaatctattacttatttgtaattgtttctttgtttgtaaattgtgagtatcttattttatggaattttctgattttattttgaaaacaaatcaatgatttgtaagatccatctgtattatactcccttcgtctcattttatgtgtcacctgtcggatttcgagattcaaacaaatctatctttgatcgtaaatttttaatagatcttttaaacattttgaattatcaattattgtgactttagtggctagactagtggatccgatatcgcccagcttcacgctgccgcaagcactcagggcgcaagggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatcatgagcggagaattaagggagtcacgttatgacccccgccgatgacgcgggacaagccgttttacgtttggaactgacagaaccgcaacgttgaaggagccactcagccgcgggtttctggagtttaatgagctaagcacatacgtcagaaaccattattgcgcgttcaaaagtcgcctaaggtcactatcagctagcaaatatttcttgtcaaaaatgctccactgacgttccataaattcccctcggtatccaattagagtctcatattcactctcaatccagatccccgggtaccatggcggcggcaacaacaacaacaacaacatcttcttcgatctccttctccaccaaaccatctccttcctcctccaaatcaccattaccaatctccagattctccctcccattctccctaaaccccaacaaatcatcctcctcctcccgccgccgcggtatcaaatccagctctccctcctccatctccgccgtgctcaacacaaccaccaatgtcacaaccactccctctccaaccaaacctaccaaacccgaaacattcatctcccgattcgctccagatcaaccccgcaaaggcgctgatattctcgtcgaggctttagaacgtcaaggcgtagaaaccgtattcgcttaccctggaggtgcatcaatggagattcaccaagccttaacccgctcttcctcaatccgtaacgtccttcctcgtcacgaacaaggaggtgtattcgcagcagaaggatacgctcgatcctcaggtaaaccaggtatctgtatagccacttcaggtcccggagctacaaatctcgttagcggattagccgatgcgttgttagatagtgttcctcttgtagcaatcacaggacaagtccctcgtcgtatgattggtacagatgcgtttcaagagactccgattgttgaggtaacgcgttcgattacgaagcataactatcttgtgatggatgttgaagatattcctaggattattgaggaggctttctttttagctacttctggtagacctggacctgttttggttgatgttcctaaagatattcaacaacagcttgcgattcctaattgggaacaggctatgagattacctggttatatgtctaggatgcctaaacctccggaagattctcatttggagcagattgttaggttgatttctgagtctaagaagcctgtgttgtatgttggtggtggttgtttgaactctagcgatgaattgggtaggtttgttgagcttacgggaatccctgttgcgagtacgttgatggggctgggatcttatccttgtgatgatgagttgtcgttacatatgcttggaatgcatgggactgtgtatgcaaattacgctgtggagcatagtgatttgttgttggcgtttggggtaaggtttgatgatcgtgtcacgggtaaacttgaggcttttgctagtagggctaagattgttcatattgatattgactcggctgagattgggaagaataagactcctcatgtgtctgtgtgtggtgatgttaagctggctttgcaagggatgaataaggttcttgagaaccgagcggaggagcttaaacttgattttggagtttggaggaatgagttgaacgtacagaaacagaagtttccgttgagctttaagacgtttggggaagctattcctccacagtatgcgattaaggtccttgatgagttgactgatggaaaagccataataagtactggtgtcgggcaacatcaaatgtgggcggcgcagttctacaattacaagaaaccaaggcagtggctatcatcaggaggccttggagctatgggatttggacttcctgctgcgattggagcgtctgttgctaaccctgatgcgatagttgtggatattgacggagatggaagttttataatgaatgtgcaagagctagccactattcgtgtagagaatcttccagtgaaggtacttttattaaacaaccagcatcttggcatggttatgcaatgggaagatcggttctacaaagctaaccgagcacacacatttctcggagatccggctcaggaggacgagatattcccgaacatgttgctgtttgcagcagcttgcgggattccagcggcgagggtgacaaagaaagcagatctccgagaagctattcagacaatgctggatacaccaggaccttacctgttggatgtgatttgtccgcaccaagaacatgtgttgccgatgatcccgaatggtggcactttcaacgatgtcataacggaaggagatggccggattaaatactgagagctcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattcactggccgtcgttttacaacgactcagctgcttggtaataattgtcattagattgtttttatgcatagatgcactcgaaatcagccaattttagacaagtatcaaacggatgttaattcagtacattaaagacgtccgcaatgtgttattaagttgtctaagttgtccttggttttattagttgttattgttgttttgtcttttcaatttctagtagtcaattgggaactggaagtacaaagttgaatttttagacttatgtatacaaattgaaaatgtatcaaaaagtagtgacacttataaaaagaacaattctttcttctgagaacatcttcaacccacctctattttactctccattctctatatttagtgagtaaaatagagaatgggcactccaacccacctccatatcactctccattcttcatatttagagagtcatattatttttattattatttttattactttctaattaatatattattttacatataatgtcattaattaaatatctaatattcataattctttttaaatgtcatattatattttaaaaaatatattatttaatatatactactttcatcccgaattaatagtattttaattttttctaattttcgacaataatataatttgattttattattaattttcactataaataatacacaaaattaaaatgataagatgaaaaaattaatttaaaaatacaatacataatatgataatttaaatacaggataacaatacaatacatgacataataatttaaatacaagataaaatacaatacataacataataatcaattcgtgatgaaacaagaatcatgccaaaaagatattgaacgtgcattcgaagttttgcaatcacgttttgcaattattgcaagaccgtcacgtttttggagaaaggaagtgtgacatgatataatgactacatgtattatactgcacaccatgataattgaggatgaacatgatcttaatgcaccaaatcaagatgccgtagaggctccaactccaacgacaaaaatgatggtagatgaaaatcttcggtttgaacaatttttagctagacataaaaaagttaaggacaaaaatgctcattttgaactccgtaatgcattaatagagcatttatgacagcaacgtgataatttcgaaacttgagtgtttatgtaattatatttcacttttatttgaatttgtccattaatttgtatattatataatatttatttacaatttatgttatttaaaaatttagaataaaataaaattttgaaaaaataaaattttatatcacatgaaaattatataaagtaattattggggaaaaaaataaaggatttatattatgaaataagaaaataagaatgaatagaaataataataatataatattgaggagaaataattattttttttagagagtgaaatagagaattgggttgaaattgattgtctgaaaaaataaaaaactctatatttggagagtaaaatagagtgtagggttggagacgtca

SEQ ID NO: 2 - DNA, 16363 bp, Artificial sequence
  synthetic construct sequence
caatttcacacaggaaacagctatgaccatgattacgccaagctggcgcgccaagcttgcatgcctgcaggtcgactctagaggatctagaatcaccgaacctcccctcggtacagctcctccagttctaccatgaatttcatccactgattcctcttcaatcgccattgcagattctctcgatctatgctcaaaaaatcccgagataaaaccctagatctgcttcaaatgctctgataccatgtaatttcagtgaattctaactaaacaatggagagaattaactattttagaaagactgattgaaggagaagaagagagaaaaattctatattgaactcatgaaccaaaatgaatgaaaaaaataatgagaagaactatactattacaatctatatatctctatttatattctaatctgaagcagttaatttaactgactctaacaactagactgataggtgtacattttctgttagtgcactgcagtgcatttaactaactgcttaacataaagaatgttgttcgaacttcattcgaatagcttcaatgagaagcaaacatgtgtacctgtaaagacacacagtaaaagtgttaataatgaataaatatgaataaatcaaataataaattaaaaataaaaacacatccaattaacattggaggtcttgaaaatcgatggtaattaacaaagacccttgtgaaatttaagtctgtaattgaaaatttgagtataggttaggggacatttgactattttctcattttctttatctttttcctaatttgtggcagacaagtgaggaggccccactgtaattgattcatgcttttgctttcttgactttttggaacaatactatgcatcatatttggtcttaattattcctctgtttatttccagaattttgagctctatacatctaataacaaagcaagcagaggatatatagtttcatcaactaaaaaggttagtcaactcatctaatatttgctactctcatctctattgaagtacagttatggaaaagtagaagtgatgtaagaaaaatgaaagaactttagtaggttagttggatctaacaaagagaaagggaaataaattgcaggagaaagagagaggttaaatacttactcacaccaccgatttacaacaaatcacttaattgtggttagttaatgtatactttcacctcattaaattattacttacccatgataagttgtattaatttggtattaatatccggtgcgggtgaattcttaccgggtgagagggatggggttggagagtgtggagtgaacagaagcagatgttttagattttttctaagatgacgaaagattcccctcactaatgaaaatatattactatacgctattagagatagaaaggttcggtaccagttggtctcgtttctggatgaaccccatttttacaagtcattttcttcaattcaaatcgcaagtgtacctttatcatcttccactaattaagtcctcttaagttcgcgtgaaaatagtgaaattattgattattcttatcatttcatcttctttctcctgataaagttttatgtactttttatgcatcaggtcttgagaacttggaaaggaaaagtagaatcatggaaaaacgaaaagataatgaagaagcaaacaactcattggtatgttatttgatagagtgaactgtaaagtattgaattgtagatatcatgtggctttaaaaatttgatatgtgttattttggcaggagtcattttctgctcttcgcaaggatgctgccaatgttctggatttcctagagagattaaagaatgaagaagatcaaaaggctgttgatgtggatctgattgaaagcctgaaattgaagctgacatttatttgtacatatgtccagctttcttattccgatttggagaagtttgaagatataatgactagaaaaagacaagaggttgagaatctgcttcaaccaattttggatgatgatggcaaagacgtcgggtgtaaatatgtccttactagcctcgccggtaatatggatgactgtataagcttgtatcatcgttctaaatcagatgccaccatgatggatgagcaattgggcttcctcctcttgaatctctctcatctatccaagcatcgtgctgaaaagatgtttcctggagtgactcaatatgaggttcttcagaatgtatgtggcaacataagagatttccatggattgatagtgaattgttgcattaagcatgagatggttgagaatgtcttatctctgtttcaactgatggctgagagagtaggacgcttcctttgggaggatcaggctgatgaagactctcaactctccgagctagatgaggatgatcagaatgataaagaccctcaactcttcaagctagcacatctactcttgaagattgttccaactgaattggaggttatgcacatatgttataaaactttgaaagcttcaacttcaacagaaattggacgcttcattaagaagctcctggaaacctctccggacattctcagagaatatctgattcatctacaagagcatatgataactgttattacccctaacacttcaggggctcgaaacattcatgtcatgatggaattcctattgattattctttctgatatgccgcccaaggactttattcatcatgacaaactttttgatctcttggctcgtgttgtagcacttaccagggaggtatcaactcttgtacgcgacttggaagagaaattaaggattaaagagagtactgacgaaacaaattgtgcaaccctaaagtttctggaaaatattgaactccttaaggaagatctcaaacatgtttatctgaaagtcccggattcatctcaatattgcttccccatgagtgatggacctctcttcatgcatctgctacagagacacttagatgatttgctggattccaatgcttattcaattgctttgataaaggaacaaattgggctggtgaaagaagacttggaattcataagatcttttttcgcgaatattgagcaaggattgtataaagatctctgggaacgtgttctagatgtggcatatgaggcaaaagatgtcatagattcaattattgttcgagataatggtctcttacatcttattttctcacttcccattaccagaaagaagatgatgcttatcaaagaagaggtctctgatttacatgagaacatttccaagaacagaggtctcatcgttgtgaactctcccaagaaaccagttgagagcaagtcattgacaactgataaaataattgtaggttttggtgaggagacaaacttgatacttagaaagctcaccagtggaccggcagatctagatgtcatttcgatcattggtatgccgggtttaggtaaaactactttggcgtacaaagtatacaatgataaatcagtttctagccatttcgaccttcgtgcatggtgcacggtcgaccaagtatatgacgagaagaagttgttggataaaattttcaatcaagttagtgactcaaattcaaaattgagtgagaatattgatgttgctgataaactacggaaacaattgtttggaaagaggtatcttattgtcttagatgacgtgtgggatactaatacatgggatgagctaacaagaccttttcctgatggtatgaaaggaagtagaattattttgacaactcgagaaaagaaagttgctttgcatggaaagctctacactgatcctcttaaccttcgattgctaagatcagaagaaagttgggagttattagagaaaagggcatttggaaacgagagttgccctgatgaactattggatgttggtaaagaaatagccgaaaattgtaaagggcttcctttggtggtggatctgattgctggaatcattgctgggagggaaaagaaaaagagtgtgtggcttgaagttgtaaataatttgcattcctttattttgaagaatgaagtggaagtgatgaaagttatagaaataagttatgaccacttacctgatcacctgaagccatgcttgctgtactttgcaagtgcgccgaaggactgggtaacgacaatccatgagttgaaacttatttggggttttgaaggatttgtggaaaagacagatatgaagagtctggaagaagtggtgaaaatttatttggatgatttaatttccagtagcttggtaatttgtttcaatgagataggtgattaccctacttgccaacttcatgatcttgtgcatgacttttgtttgataaaagcaagaaaggaaaagttgtgtgatcggataagttcaagtgctccatcagatttgttgccacgtcaaattagcattgattatgatgatgatgaagagcactttgggcttaattttgtcctgttcggttcaaataagaaaaggcattccggtaaacacctctattctttgaccataaatggagatgagctggacgaccatctttctgatacatttcatctaagacacttgaggcttcttagaaccttgcacctggaatcctcttttatcatggttaaagattctttgctgaatgaaatatgcatgttgaatcatttgaggtacttaagcattgggacagaagttaaatctctgcctttgtctttctcaaacctctggaatctagaaatcttgtttgtggataacaaagaatcaaccttgatactattaccgagaatttgggatcttgtaaagttgcaagtgctgttcacgactgcttgttctttctttgatatggatgcagatgaatcaatactgatagcagaggacacaaagttagagaacttgacagcattaggggaactcgtgctttcctattggaaagatacagaggatattttcaaaaggcttcccaatcttcaagtgcttcatttcaaactcaaggagtcatgggattattcaacagagcaatattggttcccgaaattggatttcctaactgaactagaaaaactcactgtagattttgaaagatcaaacacaaatgacagtgggtcctctgcagccataaatcggccatgggattttcactttccttcgagtttgaaaagattgcaattgcatgaatttcctctgacatccgattcactatcaacaatagcgagactgctgaaccttgaagagttgtacctttatcgtacaatcatccatggggaagaatggaacatgggagaagaagacacctttgagaatctcaaatgtttgatgttgagtcaagtgattctttccaagtgggaggttggagaggaatcttttcccacgcttgagaaattagaactgtcggactgtcataatcttgaggagattccgtctagttttggggatatttattccttgaaaattatcgaacttgtaaggagccctcaacttgaaaattccgctctcaagattaaggaatatgctgaagatatgaggggaggggacgagcttcagatccttggccagaaggatatcccgttatttaagtagtttttgagcattatggttgaaaagtagattgcactttgctgggtagattgtatatggttaagaaaattctgttacagttgttatgaaacatttttatttgacttttctgagtttcttttagaaaactcagaagtttttaacaaaaattatagtttttataaatacaatgtggatttgcctttggctgtccaacttggtctgaagtctcatatgctcagagcactatcgttcaacctcaatcaaggtactgatttaaaatgacatctatactactttatcacaaacccaacgaactttcatctcaaaagctaggccaggaagtgaagaggttgtagagagcttataagcactcatgacttccttttctcgaacattcaaccaacgtaggctgaaatcccactctgaacgaaaataagtgtttgtttatcaaattaactctcgtagtagaacactgaaataccttcttctaaacgttcaacaaatgggatttccagcactcaaagtgaatgaaaggttcacattaatcttcaaaaagaattacgacaattcatgaccacaagtacattgacagcaccatttcaacagaagaacaagtcaatgctgcatcttcatcaataatccgagtgtcgaacctccttcctgacactgtcctgtatatgtaaagtttctcaacagggcaactttctggtctcgtatctggatgacccctctcgtctataacttcaacattaagccctggcaacttctggaccaacagcttacatgcttcaaaacttactgaacaattagacatccaaagggatcgcattgtctccagctttgcagcattagccaacagagcctcatcgccaaaggggcagtctctaatctcgaatttgaaaaaattgttgttgtatgactttcctctgacatccgatgcactatcaacaatagcaagactggaggttggagaggaatcctttattatacaatcattcagggagaagaatggaacatgggggaggaagacacttttgagaatctgaaatgtgttagagccacaagctacagaagtattgaatttgtcatgaatatcaacattcttcatcctagttaattctttttcaatttttaatagactctcattttaatcactaatattcttctatttgtgacttcttttctgcaggtggcaactttaaattcataaagtataggattgatgacaaactcgaaaaatatcttaatgaggtgaagtttgagcagtcagcagatggtggttccaactctaagttgacaagcacatactatcccggagggcgatttcaagcctgatgcatatggttagtgtggctagagcagacaggatgtattacctggatatctaccaagacgaatccacaatcagttttatgtcaagcaatacatgaagtaactcccgatagaacagtaaaagcaagatgtgtaggtgtatctcgactctaagagattgtacattcctctttgagatttttactgctaatacaaatttacacctcagaagcgaatctagaatttctagagcatgaatgcaccactaatgaaaggagaaaaaaggaagtatgaagtgggaatttgatccttgtttctaggtatataaaatttatcattcaactatacttcatttagcaaacaactctctttgccattatttctcaaacaagggcttctaatattgctaaactaaagactgtcaaaaggtaagttcatcttcaaactctcttgtttactttatctaaaggggaactatgaaaaacaagaaacatcaggaatgtcccgtaaacaaagcagcctcatgcacaaaacatccaacgttggtaggattaatggagggatcgcatcccaggaggatactgtagaaaaattagtggcttctttcaccgctcaaacccatgatctataggttacatggagacaactttatggttgctcgtaggctcccgtcaattctcataaaccacaacaccaaagttgcatcagacatcatcttcattcacaagctgacaatctccacaagtcttagtcaacttgtaatatgaatattagccaggtagacgtacatatttacaaaattgagtttcctatataatatggtttgaaggaatgaaacatgatggggagggtagataaaataatatatgaggcataaaaataggaaagatatttgtagtgagaggttttgactttttatgctgcttttgatcttcagtttcttgtattctttttctactgctttcctcttctttctcctgagtaaagttttatgtaggtactttttatacgtccgatcgtgagaacttgaaagaaagctctctatagctatgttaggtgcccacataaaaaaatgaaatattacaaaaaccctgataataaaatacactaatctaagatattcactgcaacatacatgcaaaatatatatatataaattttcatgaaaattataacaaataatagatgtgaacatataactttaaaaataatattacatccataaagcttaaattctagatccccggtcgactctagaggatccccactccatccgttcactttgatttgtcatgttgcacttttcgaaagtcaatttgactaatttttaaagctaaattagattacactaattcaatattttaaacagaaaaattagatattcaaaaactatacaaaaaatattatacattgcaattttttgcatatcaatatgataaaaaaatatatcgtaaaatattagtcaaaatttttataatttgactcaaatcatgaaaagtataataattaatagtggacggaggaagtattgtctttccagatttgtggccatttttggtccaagggccattagcagttctcttcattttctacttctgtctcatattagatgggcatcttactaaaaatatttgtctcatattacttgattatttattaaatcaaaaagaattaattaattttttctcattttacccctacaattaatatagttttaaaagttttaaacaaattttgaagaatcaaaatttctttttgcaagagacttattaatataaacaaaggataaaataataaaatttgtcaatttattgacgatcacttaataatcatataaaatagaaaatgtttatctaatatgagacggagaaaatatatcctaaaatatttttggacagatatgtgatattctaaccattcactagactatattatgcattttagccgccaatgacttatttcagctttaattaattaggaaagaggaaactgccaatgaggaagagtaggggcgtagttgctgtcgacgaaaaaaagataatactcactcttttcgatttttatttttatttatcacttttaacctatcatgtaaaaagataattatttttttcatgctttatccttagtattaaacaatttaatagggattattttgtaaaatatttatatgaataattgttttcgtaatgaatttgtccggtcaaacaatgataaataaaaatgaatgaagagagtagaaaacaaaacaaaagaacaagttgacaacttgagagattaaaagggtccaaaacgccttggattttgagattccatatgtgaaatttccatgaaataattgaatttgtattattacaagtcaaactttccatttcattccaactagccatcttggtttcaaaattacacattcattcattcacagatctaatattcttaatagtgatttccacatatggctgaagctttcattcaagttctgctagacaatctcacttctttcctcaaaggggaacttgtattgcttttcggttttcaagatgagttccaaaggctttcaagcatgttttctacaattcaagccgtccttgaagatgctcaggagaagcaactcaacaacaagcctctagaaaattggttgcaaaaactcaatgctgctacatatgaagtcgatgacatcttggatgaatataaaaccaaggccacaagattctcccagtctgaatatggccgttatcatccaaaggttatccctttccgtcacaaggtcgggaaaaggatggaccaagtgatgaaaaaactaaaggcaattgctgaggaaagaaagaattttcatttgcacgaaaaaattgtagagagacaagctgttagacgggaaacaggtactcatcttaaattagtattacaacaactaagtttatattcatttttttggcaattatcaaattcagaaaagggttaaatatactcatgtcctatcgtaaatagtgtatatatacctctcgttgtactttcgatctgaatatacttgtcaaatctggcaagctcagaatcaaattatccaccccaacttttaaatactcgatatctttagaaatccacctgtctaactcatccactacccattccctttgctttgaattcttttctttacctataaacttggaacactcgatccgttttgcttttcttaacaaagcagctcagagaaaagaggttttcttctattctgtttctctgtgtgctgcacttgggtccttaatcccattaaaaacagggcatgttaatcccaacgacggtagcctttcctgacagctgactgtaaattttgtctaacaaagaaaaaaaaagattagacatgtttttccttgtcattgattaggctggatttctttcagagtggaacataggggatatattggaccaaaagtagaatgggtatatatttaaagtatttctgatagaacaggagtatattgtgcgaaaatatcctctattttctgttgtctcctaatgagtttgaatgtaataatattctcatgtggacattgcttgcaccaggttctgtattaaccgaaccgcaggtttatggaagagacaaagagaaagatgagatagtgaaaatcctaataaacaatgttagtgatgcccaacacctttcagtcctcccaatacttggtatggggggattaggaaaaacgactcttgcccaaatggtcttcaatgaccagagagttactgagcatttccattccaaaatatggatttgtgtctcggaagattttgatgagaagaggttaataaaggcaattgtagaatctattgaaggaaggccactacttggtgagatggacttggctccacttcaaaagaagcttcaggagttgctgaatggaaaaagatacttgcttgtcttagatgatgtttggaatgaagatcaacagaagtgggctaatttaagagcagtcttgaaggttggagcaagtggtgcttctgttctaaccactactcgtcttgaaaaggttggatcaattatgggaacattgcaaccatatgaactgtcaaatctgtctcaagaagattgttggttgttgttcatgcaacgtgcatttggacaccaagaagaaataaatccaaaccttgtggcaatcggaaaggagattgtgaaaaaaagtggtggtgtgcctctagcagccaaaactcttggaggtattttgtgcttcaagagagaagaaagagcatgggaacatgtgagagacagtccgatttggaatttgcctcaagatgaaagttctattctgcctgccctgaggcttagttaccatcaacttccacttgatttgaaacaatgctttgcgtattgtgcggtgttcccaaaggatgccaaaatggaaaaagaaaagctaatctctctctggatggcgcatggttttcttttatcaaaaggaaacatggagctagaggatgtgggcgatgaagtatggaaagaattatacttgaggtcttttttccaagagattgaagttaaagatggtaaaacttatttcaagatgcatgatctcatccatgatttggcaacatctctgttttcagcaaacacatcaagcagcaatatccgtgaaataaataaacacagttacacacatatgatgtccattggtttcgccgaagtggtgtttttttacactcttccccccttggaaaagtttatctcgttaagagtgcttaatctaggtgattcgacatttaataagttaccatcttccattggagatctagtacatttaagatacttgaacctgtatggcagtggcatgcgtagtcttccaaagcagttatgcaagcttcaaaatctgcaaactcttgatctacaatattgcaccaagctttgttgtttgccaaaagaaacaagtaaacttggtagtctccgaaatcttttacttgatggtagccagtcattgacttgtatgccaccaaggataggatcattgacatgccttaagactctaggtcaatttgttgttggaaggaagaaaggttatcaacttggtgaactaggaaacctaaatctctatggctcaattaaaatctcgcatcttgagagagtgaagaatgataaggacgcaaaagaagccaatttatctgcaaaagggaatctgcattctttaagcatgagttggaataactttggaccacatatatatgaatcagaagaagttaaagtgcttgaagccctcaaaccacactccaatctgacttctttaaaaatctatggcttcagaggaatccatctcccagagtggatgaatcactcagtattgaaaaatattgtctctattctaattagcaacttcagaaactgctcatgcttaccaccctttggtgatctgccttgtctagaaagtctagagttacactgggggtctgcggatgtggagtatgttgaagaagtggatattgatgttcattctggattccccacaagaataaggtttccatccttgaggaaacttgatatatgggactttggtagtctgaaaggattgctgaaaaaggaaggagaagagcaattccctgtgcttgaagagatgataattcacgagtgcccttttctgaccctttcttctaatcttagggctcttacttccctcagaatttgctataataaagtagctacttcattcccagaagagatgttcaaaaaccttgcaaatctcaaatacttgacaatctctcggtgcaataatctcaaagagctgcctaccagcttggctagtctgaatgctttgaaaagtctaaaaattcaattgtgttgcgcactagagagtctccctgaggaagggctggaaggtttatcttcactcacagagttatttgttgaacactgtaacatgctaaaatgtttaccagagggattgcagcacctaacaaccctcacaagtttaaaaattcggggatgtccacaactgatcaagcggtgtgagaagggaataggagaagactggcacaaaatttctcacattcctaatgtgaatatatatatttaagttatttgctattgtttctttgtttgtgagtctttttggttcctgccattgtgattgcatgtaatttttttctagggttgtttctttatgagtctctctctcattggatgtaattttcttttggaaacaaatctgtcaattgatttgtattatacgctttcagaatctattacttatttgtaattgtttctttgtttgtaaattgtgagtatcttattttatggaattttctgattttattttgaaaacaaatcaatgatttgtaagatccatctgtattatactcccttcgtctcattttatgtgtcacctgtcggatttcgagattcaaacaaatctatctttgatcgtaaatttttaatagatcttttaaacattttgaattatcaattattgtgactttagtggctagactagtggatccgatatcgcccagcttcacgctgccgcaagcactcagggcgcaagggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatcatgagcggagaattaagggagtcacgttatgacccccgccgatgacgcgggacaagccgttttacgtttggaactgacagaaccgcaacgttgaaggagccactcagccgcgggtttctggagtttaatgagctaagcacatacgtcagaaaccattattgcgcgttcaaaagtcgcctaaggtcactatcagctagcaaatatttcttgtcaaaaatgctccactgacgttccataaattcccctcggtatccaattagagtctcatattcactctcaatccagatccccgggtaccatggcggcggcaacaacaacaacaacaacatcttcttcgatctccttctccaccaaaccatctccttcctcctccaaatcaccattaccaatctccagattctccctcccattctccctaaaccccaacaaatcatcctcctcctcccgccgccgcggtatcaaatccagctctccctcctccatctccgccgtgctcaacacaaccaccaatgtcacaaccactccctctccaaccaaacctaccaaacccgaaacattcatctcccgattcgctccagatcaaccccgcaaaggcgctgatattctcgtcgaggctttagaacgtcaaggcgtagaaaccgtattcgcttaccctggaggtgcatcaatggagattcaccaagccttaacccgctcttcctcaatccgtaacgtccttcctcgtcacgaacaaggaggtgtattcgcagcagaaggatacgctcgatcctcaggtaaaccaggtatctgtatagccacttcaggtcccggagctacaaatctcgttagcggattagccgatgcgttgttagatagtgttcctcttgtagcaatcacaggacaagtccctcgtcgtatgattggtacagatgcgtttcaagagactccgattgttgaggtaacgcgttcgattacgaagcataactatcttgtgatggatgttgaagatattcctaggattattgaggaggctttctttttagctacttctggtagacctggacctgttttggttgatgttcctaaagatattcaacaacagcttgcgattcctaattgggaacaggctatgagattacctggttatatgtctaggatgcctaaacctccggaagattctcatttggagcagattgttaggttgatttctgagtctaagaagcctgtgttgtatgttggtggtggttgtttgaactctagcgatgaattgggtaggtttgttgagcttacgggaatccctgttgcgagtacgttgatggggctgggatcttatccttgtgatgatgagttgtcgttacatatgcttggaatgcatgggactgtgtatgcaaattacgctgtggagcatagtgatttgttgttggcgtttggggtaaggtttgatgatcgtgtcacgggtaaacttgaggcttttgctagtagggctaagattgttcatattgatattgactcggctgagattgggaagaataagactcctcatgtgtctgtgtgtggtgatgttaagctggctttgcaagggatgaataaggttcttgagaaccgagcggaggagcttaaacttgattttggagtttggaggaatgagttgaacgtacagaaacagaagtttccgttgagctttaagacgtttggggaagctattcctccacagtatgcgattaaggtccttgatgagttgactgatggaaaagccataataagtactggtgtcgggcaacatcaaatgtgggcggcgcagttctacaattacaagaaaccaaggcagtggctatcatcaggaggccttggagctatgggatttggacttcctgctgcgattggagcgtctgttgctaaccctgatgcgatagttgtggatattgacggagatggaagttttataatgaatgtgcaagagctagccactattcgtgtagagaatcttccagtgaaggtacttttattaaacaaccagcatcttggcatggttatgcaatgggaagatcggttctacaaagctaaccgagcacacacatttctcggagatccggctcaggaggacgagatattcccgaacatgttgctgtttgcagcagcttgcgggattccagcggcgagggtgacaaagaaagcagatctccgagaagctattcagacaatgctggatacaccaggaccttacctgttggatgtgatttgtccgcaccaagaacatgtgttgccgatgatcccgaatggtggcactttcaacgatgtcataacggaaggagatggccggattaaatactgagagctcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattcactggccgtcgttttacaacgactcagctgcttggtaataattgtcattagattgtttttatgcatagatgcactcgaaatcagccaattttagacaagtatcaaacggatgttaattcagtacattaaagacgtccgcaatgtgttattaagttgtctaag

SEQ ID NO: 3 - DNA, 13779 bp, Artificial sequence
  synthetic construct sequence
caatttcacacaggaaacagctatgaccatgattacgccaagctggcgcgccaagcttgcatgcctgcaggtcgactctagaggatctagaatcaccgaacctcccctcggtacagctcctccagttctaccatgaatttcatccactgattcctcttcaatcgccattgcagattctctcgatctatgctcaaaaaatcccgagataaaaccctagatctgcttcaaatgctctgataccatgtaatttcagtgaattctaactaaacaatggagagaattaactattttagaaagactgattgaaggagaagaagagagaaaaattctatattgaactcatgaaccaaaatgaatgaaaaaaataatgagaagaactatactattacaatctatatatctctatttatattctaatctgaagcagttaatttaactgactctaacaactagactgataggtgtacattttctgttagtgcactgcagtgcatttaactaactgcttaacataaagaatgttgttcgaacttcattcgaatagcttcaatgagaagcaaacatgtgtacctgtaaagacacacagtaaaagtgttaataatgaataaatatgaataaatcaaataataaattaaaaataaaaacacatccaattaacattggaggtcttgaaaatcgatggtaattaacaaagacccttgtgaaatttaagtctgtaattgaaaatttgagtataggttaggggacatttgactattttctcattttctttatctttttcctaatttgtggcagacaagtgaggaggccccactgtaattgattcatgcttttgctttcttgactttttggaacaatactatgcatcatatttggtcttaattattcctctgtttatttccagaattttgagctctatacatctaataacaaagcaagcagaggatatatagtttcatcaactaaaaaggttagtcaactcatctaatatttgctactctcatctctattgaagtacagttatggaaaagtagaagtgatgtaagaaaaatgaaagaactttagtaggttagttggatctaacaaagagaaagggaaataaattgcaggagaaagagagaggttaaatacttactcacaccaccgatttacaacaaatcacttaattgtggttagttaatgtatactttcacctcattaaattattacttacccatgataagttgtattaatttggtattaatatccggtgcgggtgaattcttaccgggtgagagggatggggttggagagtgtggagtgaacagaagcagatgttttagattttttctaagatgacgaaagattcccctcactaatgaaaatatattactatacgctattagagatagaaaggttcggtaccagttggtctcgtttctggatgaaccccatttttacaagtcattttcttcaattcaaatcgcaagtgtacctttatcatcttccactaattaagtcctcttaagttcgcgtgaaaatagtgaaattattgattattcttatcatttcatcttctttctcctgataaagttttatgtactttttatgcatcaggtcttgagaacttggaaaggaaaagtagaatcatggaaaaacgaaaagataatgaagaagcaaacaactcattggtatgttatttgatagagtgaactgtaaagtattgaattgtagatatcatgtggctttaaaaatttgatatgtgttattttggcaggagtcattttctgctcttcgcaaggatgctgccaatgttctggatttcctagagagattaaagaatgaagaagatcaaaaggctgttgatgtggatctgattgaaagcctgaaattgaagctgacatttatttgtacatatgtccagctttcttattccgatttggagaagtttgaagatataatgactagaaaaagacaagaggttgagaatctgcttcaaccaattttggatgatgatggcaaagacgtcgggtgtaaatatgtccttactagcctcgccggtaatatggatgactgtataagcttgtatcatcgttctaaatcagatgccaccatgatggatgagcaattgggcttcctcctcttgaatctctctcatctatccaagcatcgtgctgaaaagatgtttcctggagtgactcaatatgaggttcttcagaatgtatgtggcaacataagagatttccatggattgatagtgaattgttgcattaagcatgagatggttgagaatgtcttatctctgtttcaactgatggctgagagagtaggacgcttcctttgggaggatcaggctgatgaagactctcaactctccgagctagatgaggatgatcagaatgataaagaccctcaactcttcaagctagcacatctactcttgaagattgttccaactgaattggaggttatgcacatatgttataaaactttgaaagcttcaacttcaacagaaattggacgcttcattaagaagctcctggaaacctctccggacattctcagagaatatctgattcatctacaagagcatatgataactgttattacccctaacacttcaggggctcgaaacattcatgtcatgatggaattcctattgattattctttctgatatgccgcccaaggactttattcatcatgacaaactttttgatctcttggctcgtgttgtagcacttaccagggaggtatcaactcttgtacgcgacttggaagagaaattaaggattaaagagagtactgacgaaacaaattgtgcaaccctaaagtttctggaaaatattgaactccttaaggaagatctcaaacatgtttatctgaaagtcccggattcatctcaatattgcttccccatgagtgatggacctctcttcatgcatctgctacagagacacttagatgatttgctggattccaatgcttattcaattgctttgataaaggaacaaattgggctggtgaaagaagacttggaattcataagatcttttttcgcgaatattgagcaaggattgtataaagatctctgggaacgtgttctagatgtggcatatgaggcaaaagatgtcatagattcaattattgttcgagataatggtctcttacatcttattttctcacttcccattaccagaaagaagatgatgcttatcaaagaagaggtctctgatttacatgagaacatttccaagaacagaggtctcatcgttgtgaactctcccaagaaaccagttgagagcaagtcattgacaactgataaaataattgtaggttttggtgaggagacaaacttgatacttagaaagctcaccagtggaccggcagatctagatgtcatttcgatcattggtatgccgggtttaggtaaaactactttggcgtacaaagtatacaatgataaatcagtttctagccatttcgaccttcgtgcatggtgcacggtcgaccaagtatatgacgagaagaagttgttggataaaattttcaatcaagttagtgactcaaattcaaaattgagtgagaatattgatgttgctgataaactacggaaacaattgtttggaaagaggtatcttattgtcttagatgacgtgtgggatactaatacatgggatgagctaacaagaccttttcctgatggtatgaaaggaagtagaattattttgacaactcgagaaaagaaagttgctttgcatggaaagctctacactgatcctcttaaccttcgattgctaagatcagaagaaagttgggagttattagagaaaagggcatttggaaacgagagttgccctgatgaactattggatgttggtaaagaaatagccgaaaattgtaaagggcttcctttggtggtggatctgattgctggaatcattgctgggagggaaaagaaaaagagtgtgtggcttgaagttgtaaataatttgcattcctttattttgaagaatgaagtggaagtgatgaaagttatagaaataagttatgaccacttacctgatcacctgaagccatgcttgctgtactttgcaagtgcgccgaaggactgggtaacgacaatccatgagttgaaacttatttggggttttgaaggatttgtggaaaagacagatatgaagagtctggaagaagtggtgaaaatttatttggatgatttaatttccagtagcttggtaatttgtttcaatgagataggtgattaccctacttgccaacttcatgatcttgtgcatgacttttgtttgataaaagcaagaaaggaaaagttgtgtgatcggataagttcaagtgctccatcagatttgttgccacgtcaaattagcattgattatgatgatgatgaagagcactttgggcttaattttgtcctgttcggttcaaataagaaaaggcattccggtaaacacctctattctttgaccataaatggagatgagctggacgaccatctttctgatacatttcatctaagacacttgaggcttcttagaaccttgcacctggaatcctcttttatcatggttaaagattctttgctgaatgaaatatgcatgttgaatcatttgaggtacttaagcattgggacagaagttaaatctctgcctttgtctttctcaaacctctggaatctagaaatcttgtttgtggataacaaagaatcaaccttgatactattaccgagaatttgggatcttgtaaagttgcaagtgctgttcacgactgcttgttctttctttgatatggatgcagatgaatcaatactgatagcagaggacacaaagttagagaacttgacagcattaggggaactcgtgctttcctattggaaagatacagaggatattttcaaaaggcttcccaatcttcaagtgcttcatttcaaactcaaggagtcatgggattattcaacagagcaatattggttcccgaaattggatttcctaactgaactagaaaaactcactgtagattttgaaagatcaaacacaaatgacagtgggtcctctgcagccataaatcggccatgggattttcactttccttcgagtttgaaaagattgcaattgcatgaatttcctctgacatccgattcactatcaacaatagcgagactgctgaaccttgaagagttgtacctttatcgtacaatcatccatggggaagaatggaacatgggagaagaagacacctttgagaatctcaaatgtttgatgttgagtcaagtgattctttccaagtgggaggttggagaggaatcttttcccacgcttgagaaattagaactgtcggactgtcataatcttgaggagattccgtctagttttggggatatttattccttgaaaattatcgaacttgtaaggagccctcaacttgaaaattccgctctcaagattaaggaatatgctgaagatatgaggggaggggacgagcttcagatccttggccagaaggatatcccgttatttaagtagtttttgagcattatggttgaaaagtagattgcactttgctgggtagattgtatatggttaagaaaattctgttacagttgttatgaaacatttttatttgacttttctgagtttcttttagaaaactcagaagtttttaacaaaaattatagtttttataaatacaatgtggatttgcctttggctgtccaacttggtctgaagtctcatatgctcagagcactatcgttcaacctcaatcaaggtactgatttaaaatgacatctatactactttatcacaaacccaacgaactttcatctcaaaagctaggccaggaagtgaagaggttgtagagagcttataagcactcatgacttccttttctcgaacattcaaccaacgtaggctgaaatcccactctgaacgaaaataagtgtttgtttatcaaattaactctcgtagtagaacactgaaataccttcttctaaacgttcaacaaatgggatttccagcactcaaagtgaatgaaaggttcacattaatcttcaaaaagaattacgacaattcatgaccacaagtacattgacagcaccatttcaacagaagaacaagtcaatgctgcatcttcatcaataatccgagtgtcgaacctccttcctgacactgtcctgtatatgtaaagtttctcaacagggcaactttctggtctcgtatctggatgacccctctcgtctataacttcaacattaagccctggcaacttctggaccaacagcttacatgcttcaaaacttactgaacaattagacatccaaagggatcgcattgtctccagctttgcagcattagccaacagagcctcatcgccaaaggggcagtctctaatctcgaatttgaaaaaattgttgttgtatgactttcctctgacatccgatgcactatcaacaatagcaagactggaggttggagaggaatcctttattatacaatcattcagggagaagaatggaacatgggggaggaagacacttttgagaatctgaaatgtgttagagccacaagctacagaagtattgaatttgtcatgaatatcaacattcttcatcctagttaattctttttcaatttttaatagactctcattttaatcactaatattcttctatttgtgacttcttttctgcaggtggcaactttaaattcataaagtataggattgatgacaaactcgaaaaatatcttaatgaggtgaagtttgagcagtcagcagatggtggttccaactctaagttgacaagcacatactatcccggagggcgatttcaagcctgatgcatatggttagtgtggctagagcagacaggatgtattacctggatatctaccaagacgaatccacaatcagttttatgtcaagcaatacatgaagtaactcccgatagaacagtaaaagcaagatgtgtaggtgtatctcgactctaagagattgtacattcctctttgagatttttactgctaatacaaatttacacctcagaagcgaatctagaatttctagagcatgaatgcaccactaatgaaaggagaaaaaaggaagtatgaagtgggaatttgatccttgtttctaggtatataaaatttatcattcaactatacttcatttagcaaacaactctctttgccattatttctcaaacaagggcttctaatattgctaaactaaagactgtcaaaaggtaagttcatcttcaaactctcttgtttactttatctaaaggggaactatgaaaaacaagaaacatcaggaatgtcccgtaaacaaagcagcctcatgcacaaaacatccaacgttggtaggattaatggagggatcgcatcccaggaggatactgtagaaaaattagtggcttctttcaccgctcaaacccatgatctataggttacatggagacaactttatggttgctcgtaggctcccgtcaattctcataaaccacaacaccaaagttgcatcagacatcatcttcattcacaagctgacaatctccacaagtcttagtcaacttgtaatatgaatattagccaggtagacgtacatatttacaaaattgagtttcctatataatatggtttgaaggaatgaaacatgatggggagggtagataaaataatatatgaggcataaaaataggaaagatatttgtagtgagaggttttgactttttatgctgcttttgatcttcagtttcttgtattctttttctactgctttcctcttctttctcctgagtaaagttttatgtaggtactttttatacgtccgatcgtgagaacttgaaagaaagctctctatagctatgttaggtgcccacataaaaaaatgaaatattacaaaaaccctgataataaaatacactaatctaagatattcactgcaacatacatgcaaaatatatatatataaattttcatgaaaattataacaaataatagatgtgaacatataactttaaaaataatattacatccataaagcttaaattctagatccccggtcgactctagaggatccccactccatccgttcactttgatttgtcatgttgcacttttcgaaagtcaatttgactaatttttaaagctaaattagattacactaattcaatattttaaacagaaaaattagatattcaaaaactatacaaaaaatattatacattgcaattttttgcatatcaatatgataaaaaaatatatcgtaaaatattagtcaaaatttttataatttgactcaaatcatgaaaagtataataattaatagtggacggaggaagtattgtctttccagatttgtggccatttttggtccaagggccattagcagttctcttcattttctacttctgtctcatattagatgggcatcttactaaaaatatttgtctcatattacttgattatttattaaatcaaaaagaattaattaattttttctcattttacccctacaattaatatagttttaaaagttttaaacaaattttgaagaatcaaaatttctttttgcaagagacttattaatataaacaaaggataaaataataaaatttgtcaatttattgacgatcacttaataatcatataaaatagaaaatgtttatctaatatgagacggagaaaatatatcctaaaatatttttggacagatatgtgatattctaaccattcactagactatattatgcattttagccgccaatgacttatttcagctttaattaattaggaaagaggaaactgccaatgaggaagagtaggggcgtagttgctgtcgacgaaaaaaagataatactcactcttttcgatttttatttttatttatcacttttaacctatcatgtaaaaagataattatttttttcatgctttatccttagtattaaacaatttaatagggattattttgtaaaatatttatatgaataattgttttcgtaatgaatttgtccggtcaaacaatgataaataaaaatgaatgaagagagtagaaaacaaaacaaaagaacaagttgacaacttgagagattaaaagggtccaaaacgccttggattttgagattccatatgtgaaatttccatgaaataattgaatttgtattattacaagtcaaactttccatttcattccaactagccatcttggtttcaaaattacacattcattcattcacagatctaatattcttaatagtgatttccacatatggctgaagctttcattcaagttctgctagacaatctcacttctttcctcaaaggggaacttgtattgcttttcggttttcaagatgagttccaaaggctttcaagcatgttttctacaattcaagccgtccttgaagatgctcaggagaagcaactcaacaacaagcctctagaaaattggttgcaaaaactcaatgctgctacatatgaagtcgatgacatcttggatgaatataaaaccaaggccacaagattctcccagtctgaatatggccgttatcatccaaaggttatccctttccgtcacaaggtcgggaaaaggatggaccaagtgatgaaaaaactaaaggcaattgctgaggaaagaaagaattttcatttgcacgaaaaaattgtagagagacaagctgttagacgggaaacaggtactcatcttaaattagtattacaacaactaagtttatattcatttttttggcaattatcaaattcagaaaagggttaaatatactcatgtcctatcgtaaatagtgtatatatacctctcgttgtactttcgatctgaatatacttgtcaaatctggcaagctcagaatcaaattatccaccccaacttttaaatactcgatatctttagaaatccacctgtctaactcatccactacccattccctttgctttgaattcttttctttacctataaacttggaacactcgatccgttttgcttttcttaacaaagcagctcagagaaaagaggttttcttctattctgtttctctgtgtgctgcacttgggtccttaatcccattaaaaacagggcatgttaatcccaacgacggtagcctttcctgacagctgactgtaaattttgtctaacaaagaaaaaaaaagattagacatgtttttccttgtcattgattaggctggatttctttcagagtggaacataggggatatattggaccaaaagtagaatgggtatatatttaaagtatttctgatagaacaggagtatattgtgcgaaaatatcctctattttctgttgtctcctaatgagtttgaatgtaataatattctcatgtggacattgcttgcaccaggttctgtattaaccgaaccgcaggtttatggaagagacaaagagaaagatgagatagtgaaaatcctaataaacaatgttagtgatgcccaacacctttcagtcctcccaatacttggtatggggggattaggaaaaacgactcttgcccaaatggtcttcaatgaccagagagttactgagcatttccattccaaaatatggatttgtgtctcggaagattttgatgagaagaggttaataaaggcaattgtagaatctattgaaggaaggccactacttggtgagatggacttggctccacttcaaaagaagcttcaggagttgctgaatggaaaaagatacttgcttgtcttagatgatgtttggaatgaagatcaacagaagtgggctaatttaagagcagtcttgaaggttggagcaagtggtgcttctgttctaaccactactcgtcttgaaaaggttggatcaattatgggaacattgcaaccatatgaactgtcaaatctgtctcaagaagattgttggttgttgttcatgcaacgtgcatttggacaccaagaagaaataaatccaaaccttgtggcaatcggaaaggagattgtgaaaaaaagtggtggtgtgcctctagcagccaaaactcttggaggtattttgtgcttcaagagagaagaaagagcatgggaacatgtgagagacagtccgatttggaatttgcctcaagatgaaagttctattctgcctgccctgaggcttagttaccatcaacttccacttgatttgaaacaatgctttgcgtattgtgcggtgttcccaaaggatgccaaaatggaaaaagaaaagctaatctctctctggatggcgcatggttttcttttatcaaaaggaaacatggagctagaggatgtgggcgatgaagtatggaaagaattatacttgaggtcttttttccaagagattgaagttaaagatggtaaaacttatttcaagatgcatgatctcatccatgatttggcaacatctctgttttcagcaaacacatcaagcagcaatatccgtgaaataaataaacacagttacacacatatgatgtccattggtttcgccgaagtggtgtttttttacactcttccccccttggaaaagtttatctcgttaagagtgcttaatctaggtgattcgacatttaataagttaccatcttccattggagatctagtacatttaagatacttgaacctgtatggcagtggcatgcgtagtcttccaaagcagttatgcaagcttcaaaatctgcaaactcttgatctacaatattgcaccaagctttgttgtttgccaaaagaaacaagtaaacttggtagtctccgaaatcttttacttgatggtagccagtcattgacttgtatgccaccaaggataggatcattgacatgccttaagactctaggtcaatttgttgttggaaggaagaaaggttatcaacttggtgaactaggaaacctaaatctctatggctcaattaaaatctcgcatcttgagagagtgaagaatgataaggacgcaaaagaagccaatttatctgcaaaagggaatctgcattctttaagcatgagttggaataactttggaccacatatatatgaatcagaagaagttaaagtgcttgaagccctcaaaccacactccaatctgacttctttaaaaatctatggcttcagaggaatccatctcccagagtggatgaatcactcagtattgaaaaatattgtctctattctaattagcaacttcagaaactgctcatgcttaccaccctttggtgatctgccttgtctagaaagtctagagttacactgggggtctgcggatgtggagtatgttgaagaagtggatattgatgttcattctggattccccacaagaataaggtttccatccttgaggaaacttgatatatgggactttggtagtctgaaaggattgctgaaaaaggaaggagaagagcaattccctgtgcttgaagagatgataattcacgagtgcccttttctgaccctttcttctaatcttagggctcttacttccctcagaatttgctataataaagtagctacttcattcccagaagagatgttcaaaaaccttgcaaatctcaaatacttgacaatctctcggtgcaataatctcaaagagctgcctaccagcttggctagtctgaatgctttgaaaagtctaaaaattcaattgtgttgcgcactagagagtctccctgaggaagggctggaaggtttatcttcactcacagagttatttgttgaacactgtaacatgctaaaatgtttaccagagggattgcagcacctaacaaccctcacaagtttaaaaattcggggatgtccacaactgatcaagcggtgtgagaagggaataggagaagactggcacaaaatttctcacattcctaatgtgaatatatatatttaagttatttgctattgtttctttgtttgtgagtctttttggttcctgccattgtgattgcatgtaatttttttctagggttgtttctttatgagtctctctctcattggatgtaattttcttttggaaacaaatctgtcaattgatttgtattatacgctttcagaatctattacttatttgtaattgtttctttgtttgtaaattgtgagtatcttattttatggaattttctgattttattttgaaaacaaatcaatgatttgtaagatccatctgtattatactcccttcgtctcattttatgtgtcacctgtcggatttcgagattcaaacaaatctatctttgatcgtaaatttttaatagatcttttaaacattttgaattatcaattattgtgactttagtggctagactagtggatccgatatcgcccagcttcacgctgccgcaagcactcagggcgcaagggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagggaattcactggccgtcgttttacaacgactcagctgcttggtaataattgtcattagattgtttttatgcatagatgcactcgaaatcagccaattttagacaagtatcaaacggatgttaattcagtacattaaagacgtccgcaatgtgttattaagttgtctaag

SEQ ID NO: 4 - DNA, 26 bp, Artificial sequence
  primer sequence
tcaaacggatgttaattcagtacatt

SEQ ID NO: 5 - DNA, 25 bp, Artificial sequence
  primer sequence
ccagttcccaattgactactagaaa

SEQ ID NO: 6 - DNA, 20 bp, Artificial sequence
  primer sequence
tctgttgaattacgttaagc

SEQ ID NO: 7 - DNA, 20 bp, Artificial sequence
  primer sequence
ctcagaagaaagaattgttc

SEQ ID NO: 8 - DNA, 25 bp, Artificial sequence
  primer sequence
gtttcttaagattgaatcctgttgc

SEQ ID NO: 9 - DNA, 25 bp, Artificial sequence
  primer sequence
gcccattctctattttactcactaa

SEQ ID NO: 10 - DNA, 21 bp, Artificial sequence
  primer sequence
gtgaactaggaaacctaaatc

SEQ ID NO: 11 - DNA, 20 bp, Artificial sequence
  primer sequence
caactaataaaaccaaggac

SEQ ID NO: 12 - DNA, 27 bp, Artificial sequence
  Primer
ccaagatagtgtttcaggaaagttatt

SEQ ID NO: 13 - DNA, 24 bp, Artificial sequence
  primer sequence
aaattcatggtagaactggaggag

SEQ ID NO: 14 - DNA, 20 bp, Artificial sequence
  primer sequence
aactgaattttgggattgag

SEQ ID NO: 15 - DNA, 25 bp, artificial
  primer sequence
gagtcagttaaattaactgcttcag

SEQ ID NO: 16 - DNA, 22 bp, Artificial sequence
  primer sequence
acaagaatagcaaggattatcc

SEQ ID NO: 17 - DNA, 20 bp, Artificial sequence
  primer sequence
gaagttcgaacaacattctt

SEQ ID NO: 18 - DNA, 20 bp, Artificial sequence
  primer sequence
aactgaattttgggattgag

SEQ ID NO: 19 - DNA, 20 bp, Artificial sequence
  primer sequence
atgtagcagcattgagtttt

SEQ ID NO: 20 - DNA, 985 bp, Solanum tuberosum
gaaactcagttggagattacgtttaagggatggcctacatggataaggttatctacaatgatgcaatggagataataatagtactcctatgtgatttcttcgttttaatttgtttattttattttcatttaatttgatttgatacgtagtttaaaaaaataaaaaagatatttaaattttgtgatgttagatgaaacatacgttaaatgcatcaaaatgtcatttaaatttgtcttgaacatatagtattagaaagttgaaattaaagagctatcaaaaaagaagaagatatgactaaaaaaaatagcacaagcaaattgaaactaaaagagtaattaacaataacaatttttttttaaaatgatacataatactcactctttctcaatatgtggcacaaaaatatcaagaatcaaactatttaattttgactataaatttagaaataaattttttaaatttcttaaaaaacaaaatttacaaagcaaatactatataaaaaatattataaattataataattaataattcaaaatagttaaccatacaaactgaattttgggattgagtttctatttaaattttcgtgagtaaagattaaagttcaaattaactttttataaaatgagttttcaattttaaaaagtcaaacaaaatatatatagaatataaagtgagaaaccaaataaaacaagaatagcaaggattatccatttaggaaataagatgaaagaatcaaatcaactaataaaaactttaatcctataagataagcttgccaataatttaaaatagcatgatgcgtctaagagggtatcaatatattgttatcctttaaagaccaagatagtgtttcaggaaagttatttcatttccaaatttaaataaaagaaaataattattcaaaattcttatgtcttttttattgatttatagtcgaagatctttcaaaaataatttttctataggtaaaaaaggataattt

SEQ ID NO: 21 - DNA, 1375 bp, Solanum tuberosum
ttgtccttggttttattagttgttattgttgttttgtcttttcaatttctagtagtcaattgggaactggaagtacaaagttgaatttttagacttatgtatacaaattgaaaatgtatcaaaaagtagtgacacttataaaaagaacaattctttcttctgagaacatcttcaacccacctctattttactctccattctctatatttagtgagtaaaatagagaatgggcactccaacccacctccatatcactctccattcttcatatttagagagtcatattatttttattattatttttattactttctaattaatatattattttacatataatgtcattaattaaatatctaatattcataattctttttaaatgtcatattatattttaaaaaatatattatttaatatatactactttcatcccgaattaatagtattttaattttttctaattttcgacaataatataatttgattttattattaattttcactataaataatacacaaaattaaaatgataagatgaaaaaattaatttaaaaatacaatacataatatgataatttaaatacaggataacaatacaatacatgacataataatttaaatacaagataaaatacaatacataacataataatcaattcgtgatgaaacaagaatcatgccaaaaagatattgaacgtgcattcgaagttttgcaatcacgttttgcaattattgcaagaccgtcacgtttttggagaaaggaagtgtgacatgatataatgactacatgtattatactgcacaccatgataattgaggatgaacatgatcttaatgcaccaaatcaagatgccgtagaggctccaactccaacgacaaaaatgatggtagatgaaaatcttcggtttgaacaatttttagctagacataaaaaagttaaggacaaaaatgctcattttgaactccgtaatgcattaatagagcatttatgacagcaacgtgataatttcgaaacttgagtgtttatgtaattatatttcacttttatttgaatttgtccattaatttgtatattatataatatttatttacaatttatgttatttaaaaatttagaataaaataaaattttgaaaaaataaaattttatatcacatgaaaattatataaagtaattattggggaaaaaaataaaggatttatattatgaaataagaaaataagaatgaatagaaataataataatataatattgaggagaaataattattttttttagagagtgaaatagagaattgggttgaaattgattgtctgaaaaaataaaaaactctatatttggagagtaaaatagagtgtagggttggagacgtca

SEQ ID NO: 22 - DNA, 1773 bp, Artificial sequence
  synthetic construct sequence
gtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattcactggccgtcgttttacaacgactcagctgcttggtaataattgtcattagattgtttttatgcatagatgcactcgaaatcagccaattttagacaagtatcaaacggatgttaattcagtacattaaagacgtccgcaatgtgttattaagttgtctaagttgtccttggttttattagttgttattgttgttttgtcttttcaatttctagtagtcaattgggaactggaagtacaaagttgaatttttagacttatgtatacaaattgaaaatgtatcaaaaagtagtgacacttataaaaagaacaattctttcttctgagaacatcttcaacccacctctattttactctccattctctatatttagtgagtaaaatagagaatgggcactccaacccacctccatatcactctccattcttcatatttagagagtcatattatttttattattatttttattactttctaattaatatattattttacatataatgtcattaattaaatatctaatattcataattctttttaaatgtcatattatattttaaaaaatatattatttaatatatactactttcatcccgaattaatagtattttaattttttctaattttcgacaataatataatttgattttattattaattttcactataaataatacacaaaattaaaatgataagatgaaaaaattaatttaaaaatacaatacataatatgataatttaaatacaggataacaatacaatacatgacataataatttaaatacaagataaaatacaatacataacataataatcaattcgtgatgaaacaagaatcatgccaaaaagatattgaacgtgcattcgaagttttgcaatcacgttttgcaattattgcaagaccgtcacgtttttggagaaaggaagtgtgacatgatataatgactacatgtattatactgcacaccatgataattgaggatgaacatgatcttaatgcaccaaatcaagatgccgtagaggctccaactccaacgacaaaaatgatggtagatgaaaatcttcggtttgaacaatttttagctagacataaaaaagttaaggacaaaaatgctcattttgaactccgtaatgcattaatagagcatttatgacagcaacgtgataatttcgaaacttgagtgtttatgtaattatatttcacttttatttgaatttgtccattaatttgtatattatataatatttatttacaatttatgttatttaaaaatttagaataaaataaaattttgaaaaaataaaattttatatcacatgaaaattatataaagtaattattggggaaaaaaataaaggatttatattatgaaataagaaaataagaatgaatagaaataataataatataatattgaggagaaataattattttttttagagagtgaaatagagaattgggttgaaattgattgtctgaaaaaataaaaaactctatatttggagagtaaaatagagtgtagggttggagacgtca

SEQ ID NO: 23 - DNA, 1519 bp, Artificial sequence
  synthetic construct sequence
gaaactcagttggagattacgtttaagggatggcctacatggataaggttatctacaatgatgcaatggagataataatagtactcctatgtgatttcttcgttttaatttgtttattttattttcatttaatttgatttgatacgtagtttaaaaaaataaaaaagatatttaaattttgtgatgttagatgaaacatacgttaaatgcatcaaaatgtcatttaaatttgtcttgaacatatagtattagaaagttgaaattaaagagctatcaaaaaagaagaagatatgactaaaaaaaatagcacaagcaaattgaaactaaaagagtaattaacaataacaatttttttttaaaatgatacataatactcactctttctcaatatgtggcacaaaaatatcaagaatcaaactatttaattttgactataaatttagaaataaattttttaaatttcttaaaaaacaaaatttacaaagcaaatactatataaaaaatattataaattataataattaataattcaaaatagttaaccatacaaactgaattttgggattgagtttctatttaaattttcgtgagtaaagattaaagttcaaattaactttttataaaatgagttttcaattttaaaaagtcaaacaaaatatatatagaatataaagtgagaaaccaaataaaacaagaatagcaaggattatccatttaggaaataagatgaaagaatcaaatcaactaataaaaactttaatcctataagataagcttgccaataatttaaaatagcatgatgcgtctaagagggtatcaatatattgttatcctttaaagaccaagatagtgtttcaggaaagttatttcatttccaaatttaaataaaagaaaataattattcaaaattcttatgtcttttttattgatttatagtcgaagatctttcaaaaataatttttctataggtaaaaaaggataatttcaatttcacacaggaaacagctatgaccatgattacgccaagctggcgcgccaagcttgcatgcctgcaggtcgactctagaggatctagaatcaccgaacctcccctcggtacagctcctccagttctaccatgaatttcatccactgattcctcttcaatcgccattgcagattctctcgatctatgctcaaaaaatcccgagataaaaccctagatctgcttcaaatgctctgataccatgtaatttcagtgaattctaactaaacaatggagagaattaactattttagaaagactgattgaaggagaagaagagagaaaaattctatattgaactcatgaaccaaaatgaatgaaaaaaataatgagaagaactatactattacaatctatatatctctatttatattctaatctgaagcagttaatttaactgactctaacaactagactgataggtgtacattttctgttagtgcactgcagtgcatttaactaactgcttaacataaagaatgttgttcgaacttc

SEQ ID NO: 24 - DNA, 957 bp, Artificial sequence
  synthetic construct sequence
agcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattcactggccgtcgttttacaacgactcagctgcttggtaataattgtcattagattgtttttatgcatagatgcactcgaaatcagccaattttagacaagtatcaaacggatgttaattcagtacattaaagacgtccgcaatgtgaactcatttgatcaagaaatgactatgggacattaaagtaaaaatcataaagacatttttttttcatattccataataaaagaataataacagaatattagtggaaaaaaatgaaaaatttctaaatgtaggggtgggggtggggaaggagatacattaggtttctaaaaataaataaaaaacgaactaaaccaagattgatcaaatcgaaaaaatgcgttaattggtttgattttaataattttaaaatgaattaaattgatttgattttagtggcaaaaagttgatctaaattgatctctgagtacctttagccccactaattccttttaagaggataaacgttttcaattgtaaaagacaattaggactaaatagctgaaagaaaggatagttttaacccaataatcaatgttaagacaatttaaatttaatataaagaagaaaaattaaaactattttcaatatgttagggacaagggaataaatcttaaaacaatcgcaatatcaaaattgggcgctaataataataacttaattaattaactattctagtaaagagtcgatatatttctaattaatcctctcagctttcctcgataagaagtagtaatggatataggattgtaaggtcatgcgagctct

SEQ ID NO: 25 - DNA, 25 bp, Artificial sequence
  primer sequence
taattcagtacattaaagacgtccg

SEQ ID NO: 26 - DNA, 23 bp, artificial
  primer sequence
gtcccatagtcatttcttgatca

SEQ ID NO: 27 - DNA, 599 bp, Artificial sequence
  synthetic construct sequence
gattttattttattaataaaaattgaaaaaaattaaatcaaactaaaacaataaattattcatataatttttataattatatatatacacacataatatattaattttacaattatttataatcatttatatactttttcaccattatcatatttgtctctgataggctaataaactatgtatttaatgttgctgtacattttctgatagtttaaactgaaggcgggaaacgacaatcagatctagtaggaaacagctatgaccatgattacgccaagcttgcatgccctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctggcgcgccaagcttgcatgcctgcaggtcgactctagaggatctagaatcaccgaacctcccctcggtacagctcctccagttctaccatgaatttcatccactgattcctcttcaatcgccattgca

SEQ ID NO: 28 - DNA, 26 bp, Artificial sequence
  primer sequence
tgtctctgataggctaataaactatg

SEQ ID NO: 29 - DNA, 21 bp, Artificial sequence
  primer sequence
tagatctgattgtcgtttccc

SEQ ID NO: 30 - DNA, 426 bp, Artificial sequence
  synthetic construct sequence
ggattaaatactgagagctcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattggatttgtaccagcaacaagaagacataaaattgcacgttttgccttttaaatgagttagttttttatttttttatccttcaaattgggtgatgtttaattttgttcttttctaatcgaacttatacctagtgatacataagttctttaaaattaaaatttagtccgagaataacttatgaaatattacgatataaggataaaacttcaaatgaccaacacaaaactcg

SEQ ID NO: 31 - DNA, 23 bp, Artificial sequence
  primer sequence
atgacgttatttatgagatgggt

SEQ ID NO: 32 - DNA, 20 bp, Artificial sequence
  primer sequence
atttaaaaggcaaaacgtgc

SEQ ID NO: 33 - DNA, 842 bp, Artificial sequence
  synthetic construct sequence
ttttttttgttatggagaaagcagagataatagttatagaattgttggttaccctttagatttcaagagtatagaatgatgtttagttcatttgagaatagcgctatagcagtatatttgcatacataaaccagaaacatacaaccatcacctatctcgaatgatcaagccaaactttccccttgcatagtgtcttggcttgttcttagtttaaaacaataatataaacaacaataaattactaatggccgaataatttatctgaattttaacaaattttattctcggcaaaaatatgatcaactttcccaacttagggctccttcccaccgtgaaatcatgtcaacaccttctcccaagggcaataacttagtttctacggcttaagcaataatgcacaaggttggagagtcaaacttttgcctttatcgataaattcaatgggaaaactaatgcaaactgtccaaaatcacccctcccaagtgtaagaactcaatgtacagaaaataggtgttttgggttattttctgcataaaattctgattcagctcaaaatagtagtccaacccgtgatagcgacaactagtctcactatagcgatctccacattctgctatagcgagacaccttttgccacctaaactccagaaaaatgtcatttgtacaacacaaactctcctttcatgtcaagttcaatttcaggagttcatgccctggcacgacaggtttcccgactggaaagcgggcagtgagcgcaacgcaattaatgtgagttagctcactcattaggcaccccaggctttacactttatgcttccggctcgtatgttgtgtggaagttg

SEQ ID NO: 34 - DNA, 23 bp, Artificial sequence
  primer sequence
ttcatgtcaagttcaatttcagg

SEQ ID NO: 35 - DNA, 21 bp, Artificial sequence
  primer sequence
actcacattaattgcgttgcg

SEQ ID NO: 36 - DNA, 1209 bp, Artificial sequence
  synthetic construct sequence
tgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattcactggccgtcgttttacaacgactcagctgcttggtaataattgtcattagattgtttttatgcatagatgcactccaaagaatgtaatattaatgtaaataattcaaagaatgtaatattaatgtaaataattcaaaggtcaaggcggcttgcttggagaaatgagagattgtttagttgactaatgatatgtttaattaagaagaccattttagcattcatttttacattttcttactatgaatagcagaacaaacgacgcaaatagaaccttttttcttcttccattttctcttaattttgacttagcaaaatgatgaggtaccacaaatcacaataattaatacttttcaaagatatcaataaattaatgatgatccttaaattttaggtaaaagtgttggaactgtcatgattcagaccgtcgtgattgacacccacactagccctccggtgggagaaccattactacaacccaaactaacaaattttatgaaaactaaagcatttaaagatatagaagcaggtataaatgacaataaaaatggagtccccataaactccaaggtttttaacaaacaactaatcaaactaatgcggaagaacaaccccaaaactaaacaacaccttaagcaaagtctgagtccgaaaagagtggacttaaacaagaaagatccatggcagcctgaaagaactgactcacccttgaatccggtcacgctcaatatccgcctaaagatgccctgtactcaacaaaaaaacaagcaagtacagtatcagtacacaaccacagagtactggtaggatcacgcgactatcctaataagtgaaacacatgcaagtaaccacaacattataaaacaagcatataccgaacatatacagtattagtcatcttttcaagatcaatgtagcatcacacgttctcaataatacacattggttatcattttagagcagcatacagtcttccaatatcaatcatcattag

SEQ ID NO: 37 - DNA, 26 bp, Artificial sequence
  primer sequence
gcttggtaataattgtcattagattg

SEQ ID NO: 38 - DNA, 23 bp, Artificial sequence
  primer sequence
gccttgacctttgaattatttac

SEQ ID NO: 39 - DNA, 1202 bp, Artificial sequence
  synthetic construct sequence
atacaccataccttaggagaaaaacacaactaacgttatttttatcctgcaaatggaaaggtctcaaccatagcaacggataggatcattgcagcctttaccagcagactgctttgagccaaaccgacccccctttgcaaggtactgctggtgatcttcctcagctcgataaaatctcacatcaaatgactcccaagtgtttagatataaattgcgtcatatttgaggggttctttaaagttgaattaacaataatatttagttgatattgtttgaacacccattccacttggcctttttaaaagttagcaaggttaattttcttttacttgaaacaattggaataaaaaaataattatttcctttgtctatatctcaatttatatgatatagtttaactatattagaaaagttaatcgtaataactcatataattgtcaaattattatacgctttgacctcaatctctccgaattacctcaagcgtttgggccctcatattttgaactcttcggctttttaacctaaaaggttcgttataattgaattatgaactcacttttatgtgaccctaacttttcctctcgttctgatgcagaattttctaactcaaatttcataatattcgattcttgttattgagtccaaatcataatatttgtttcttctattaaatctgattatgaaatctctagtcgcctcgtgaatttataggctacaatctcattttccctcaatgaatattcaattcttgttattgagttcaaatcataatattcgtttcttctattaaatctgattatgaaatctctagtcgcctcgtgaatttataggctacaatctcattttccaaacactgatagtttaaactgaaggcgggaaacgacaatcagatctagtaggaaacagctatgaccatgattacgccaagcttgcatgccctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctggcgcgccaagcttgcatgcctgcaggtcgactctagaggatctagaatcaccgaacctcccctcggtacagctcctc

SEQ ID NO: 40 - DNA, 25 bp, Artificial sequence
  primer sequence
tctgatgcagaattttctaactcaa

SEQ ID NO: 41 - DNA, 26 bp, Artificial sequence
  Primer
ttcctactagatctgattgtcgtttc

SEQ ID NO: 42 - DNA, 702 bp, Artificial sequence
  synthetic construct sequence
attaaatactgagagctcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattcactggccgtcgttttacaacgactcagctgcttggtaataattgtcattagattgtttttatgcatagatgcactcgaaatcagccaattttagacaagtatcaaacggatgttaattcagtacattaaagacgtccgcaatgtgttattaagttgtctaagttgtccttggttttattagttgttattgttgttttgtcttttcaatttctagtagtcaattgggaactggaagtacaaagttgaatttttagacttatgtatacaaattgaaaatgtatcaaaaagtagtgacacttataaaaagaacaattctttcttctgagaacatcttcaacccacctctattttactctccattctctatatttagtgagtaaaatagagaatgggcactccaacccaccttcatc

SEQ ID NO: 43 - DNA, 1137 bp, Artificial sequence
  synthetic construct sequence
aatagttaaccatacaaactgaattttgggattgagtttctatttaaattttcgtgagtaaagattaaagttcaaattaactttttataaaatgagttttcaattttaaaaagtcaaacaaaatatatatagaatataaagtgagaaaccaaataaaacaagaatagcaaggattatccatttaggaaataagatgaaagaatcaaatcaactaataaaaactttaatcctataagataagcttgccaataatttaaaatagcatgatgcgtctaagagggtatcaatatattgttatcctttaaagaccaagatagtgtttcaggaaagttatttcatttccaaatttaaataaaagaaaataattattcaaaattcttatgtcttttttattgatttatagtcgaagatctttcaaaaataatttttctataggtaaaaaaggataatttcaatttcacacaggaaacagctatgaccatgattacgccaagctggcgcgccaagcttgcatgcctgcaggtcgactctagaggatctagaatcaccgaacctcccctcggtacagctcctccagttctaccatgaatttcatccactgattcctcttcaatcgccattgcagattctctcgatctatgctcaaaaaatcccgagataaaaccctagatctgcttcaaatgctctgataccatgtaatttcagtgaattctaactaaacaatggagagaattaactattttagaaagactgattgaaggagaagaagagagaaaaattctatattgaactcatgaaccaaaatgaatgaaaaaaataatgagaagaactatactattacaatctatatatctctatttatattctaatctgaagcagttaatttaactgactctaacaactagactgataggtgtacattttctgttagtgcactgcagtgcatttaactaactgcttaacataaagaatgttgttcgaacttcattcgaatagcttcaatgagaagcaaacatgtgtacctgtaaagacacacagtaaaagtgttaataatgaataaatatgaataaatcaaataataaattaaaaataaaaacacatccaattaacattggaggtcttgaaaatcgatggtaa

SEQ ID NO: 44 - DNA, 5881 bp, Artificial sequence
  synthetic construct sequence
gtgaactaggaaacctaaatctctatggctcaattaaaatctcgcatcttgagagagtgaagaatgataaggacgcaaaagaagccaatttatctgcaaaagggaatctgcattctttaagcatgagttggaataactttggaccacatatatatgaatcagaagaagttaaagtgcttgaagccctcaaaccacactccaatctgacttctttaaaaatctatggcttcagaggaatccatctcccagagtggatgaatcactcagtattgaaaaatattgtctctattctaattagcaacttcagaaactgctcatgcttaccaccctttggtgatctgccttgtctagaaagtctagagttacactgggggtctgcggatgtggagtatgttgaagaagtggatattgatgttcattctggattccccacaagaataaggtttccatccttgaggaaacttgatatatgggactttggtagtctgaaaggattgctgaaaaaggaaggagaagagcaattccctgtgcttgaagagatgataattcacgagtgcccttttctgaccctttcttctaatcttagggctcttacttccctcagaatttgctataataaagtagctacttcattcccagaagagatgttcaaaaaccttgcaaatctcaaatacttgacaatctctcggtgcaataatctcaaagagctgcctaccagcttggctagtctgaatgctttgaaaagtctaaaaattcaattgtgttgcgcactagagagtctccctgaggaagggctggaaggtttatcttcactcacagagttatttgttgaacactgtaacatgctaaaatgtttaccagagggattgcagcacctaacaaccctcacaagtttaaaaattcggggatgtccacaactgatcaagcggtgtgagaagggaataggagaagactggcacaaaatttctcacattcctaatgtgaatatatatatttaagttatttgctattgtttctttgtttgtgagtctttttggttcctgccattgtgattgcatgtaatttttttctagggttgtttctttatgagtctctctctcattggatgtaattttcttttggaaacaaatctgtcaattgatttgtattatacgctttcagaatctattacttatttgtaattgtttctttgtttgtaaattgtgagtatcttattttatggaattttctgattttattttgaaaacaaatcaatgatttgtaagatccatctgtattatactcccttcgtctcattttatgtgtcacctgtcggatttcgagattcaaacaaatctatctttgatcgtaaatttttaatagatcttttaaacattttgaattatcaattattgtgactttagtggctagactagtggatccgatatcgcccagcttcacgctgccgcaagcactcagggcgcaagggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatcatgagcggagaattaagggagtcacgttatgacccccgccgatgacgcgggacaagccgttttacgtttggaactgacagaaccgcaacgttgaaggagccactcagccgcgggtttctggagtttaatgagctaagcacatacgtcagaaaccattattgcgcgttcaaaagtcgcctaaggtcactatcagctagcaaatatttcttgtcaaaaatgctccactgacgttccataaattcccctcggtatccaattagagtctcatattcactctcaatccagatccccgggtaccatggcggcggcaacaacaacaacaacaacatcttcttcgatctccttctccaccaaaccatctccttcctcctccaaatcaccattaccaatctccagattctccctcccattctccctaaaccccaacaaatcatcctcctcctcccgccgccgcggtatcaaatccagctctccctcctccatctccgccgtgctcaacacaaccaccaatgtcacaaccactccctctccaaccaaacctaccaaacccgaaacattcatctcccgattcgctccagatcaaccccgcaaaggcgctgatattctcgtcgaggctttagaacgtcaaggcgtagaaaccgtattcgcttaccctggaggtgcatcaatggagattcaccaagccttaacccgctcttcctcaatccgtaacgtccttcctcgtcacgaacaaggaggtgtattcgcagcagaaggatacgctcgatcctcaggtaaaccaggtatctgtatagccacttcaggtcccggagctacaaatctcgttagcggattagccgatgcgttgttagatagtgttcctcttgtagcaatcacaggacaagtccctcgtcgtatgattggtacagatgcgtttcaagagactccgattgttgaggtaacgcgttcgattacgaagcataactatcttgtgatggatgttgaagatattcctaggattattgaggaggctttctttttagctacttctggtagacctggacctgttttggttgatgttcctaaagatattcaacaacagcttgcgattcctaattgggaacaggctatgagattacctggttatatgtctaggatgcctaaacctccggaagattctcatttggagcagattgttaggttgatttctgagtctaagaagcctgtgttgtatgttggtggtggttgtttgaactctagcgatgaattgggtaggtttgttgagcttacgggaatccctgttgcgagtacgttgatggggctgggatcttatccttgtgatgatgagttgtcgttacatatgcttggaatgcatgggactgtgtatgcaaattacgctgtggagcatagtgatttgttgttggcgtttggggtaaggtttgatgatcgtgtcacgggtaaacttgaggcttttgctagtagggctaagattgttcatattgatattgactcggctgagattgggaagaataagactcctcatgtgtctgtgtgtggtgatgttaagctggctttgcaagggatgaataaggttcttgagaaccgagcggaggagcttaaacttgattttggagtttggaggaatgagttgaacgtacagaaacagaagtttccgttgagctttaagacgtttggggaagctattcctccacagtatgcgattaaggtccttgatgagttgactgatggaaaagccataataagtactggtgtcgggcaacatcaaatgtgggcggcgcagttctacaattacaagaaaccaaggcagtggctatcatcaggaggccttggagctatgggatttggacttcctgctgcgattggagcgtctgttgctaaccctgatgcgatagttgtggatattgacggagatggaagttttataatgaatgtgcaagagctagccactattcgtgtagagaatcttccagtgaaggtacttttattaaacaaccagcatcttggcatggttatgcaatgggaagatcggttctacaaagctaaccgagcacacacatttctcggagatccggctcaggaggacgagatattcccgaacatgttgctgtttgcagcagcttgcgggattccagcggcgagggtgacaaagaaagcagatctccgagaagctattcagacaatgctggatacaccaggaccttacctgttggatgtgatttgtccgcaccaagaacatgtgttgccgatgatcccgaatggtggcactttcaacgatgtcataacggaaggagatggccggattaaatactgagagctcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattcactggccgtcgttttacaacgactcagctgcttggtaataattgtcattagattgtttttatgcatagatgcactcgaaatcagccaattttagacaagtatcaaacggatgttaattcagtacattaaagacgtccgcaatgtgttattaagttgtctaagttgtccttggttttattagttgttattgttgttttgtcttttcaatttctagtagtcaattgggaactggaagtacaaagttgaatttttagacttatgtatacaaattgaaaatgtatcaaaaagtagtgacacttataaaaagaacaattctttcttctgagaacatcttcaacccacctctattttactctccattctctatatttagtgagtaaaatagagaatgggcactccaacccacctccatatcactctccattcttcatatttagagagtcatattatttttattattatttttattactttctaattaatatattattttacatataatgtcattaattaaatatctaatattcataattctttttaaatgtcatattatattttaaaaaatatattatttaatatatactactttcatcccgaattaatagtattttaattttttctaattttcgacaataatataatttgattttattattaattttcactataaataatacacaaaattaaaatgataagatgaaaaaattaatttaaaaatacaatacataatatgataatttaaatacaggataacaatacaatacatgacataataatttaaatacaagataaaatacaatacataacataataatcaattcgtgatgaaacaagaatcatgccaaaaagatattgaacgtgcattcgaagttttgcaatcacgttttgcaattattgcaagaccgtcacgtttttggagaaaggaagtgtgacatgatataatgactacatgtattatactgcacaccatgataattgaggatgaacatgatcttaatgcaccaaatcaagatgccgtagaggctccaactccaacgacaaaaatgatggtagatgaaaatcttcggtttgaacaatttttagctagacataaaaaagttaaggacaaaaatgctcattttgaactccgtaatgcattaatagagcatttatgacagcaacgtgataatttcgaaacttgagtgtttatgtaattatatttcacttttatttgaatttgtccattaatttgtatattatataatatttatttacaatttatgttatttaaaaatttagaataaaataaaattttgaaaaaataaaattttatatcacatgaaaattatataaagtaattattggggaaaaaaataaaggatttatattatgaaataagaaaataagaatgaatagaaataataataatataatattgaggagaaataattattttttttagagagtgaaatagagaattgggttgaaattgattgtctgaaaaaataaaaaactctatatttggagagtaaaatagagtgtagggttggagacgtca

SEQ ID NO: 45 - DNA, 14246 bp, Artificial sequence
  synthetic construct sequence
gaaactcagttggagattacgtttaagggatggcctacatggataaggttatctacaatgatgcaatggagataataatagtactcctatgtgatttcttcgttttaatttgtttattttattttcatttaatttgatttgatacgtagtttaaaaaaataaaaaagatatttaaattttgtgatgttagatgaaacatacgttaaatgcatcaaaatgtcatttaaatttgtcttgaacatatagtattagaaagttgaaattaaagagctatcaaaaaagaagaagatatgactaaaaaaaatagcacaagcaaattgaaactaaaagagtaattaacaataacaatttttttttaaaatgatacataatactcactctttctcaatatgtggcacaaaaatatcaagaatcaaactatttaattttgactataaatttagaaataaattttttaaatttcttaaaaaacaaaatttacaaagcaaatactatataaaaaatattataaattataataattaataattcaaaatagttaaccatacaaactgaattttgggattgagtttctatttaaattttcgtgagtaaagattaaagttcaaattaactttttataaaatgagttttcaattttaaaaagtcaaacaaaatatatatagaatataaagtgagaaaccaaataaaacaagaatagcaaggattatccatttaggaaataagatgaaagaatcaaatcaactaataaaaactttaatcctataagataagcttgccaataatttaaaatagcatgatgcgtctaagagggtatcaatatattgttatcctttaaagaccaagatagtgtttcaggaaagttatttcatttccaaatttaaataaaagaaaataattattcaaaattcttatgtcttttttattgatttatagtcgaagatctttcaaaaataatttttctataggtaaaaaaggataatttcaatttcacacaggaaacagctatgaccatgattacgccaagctggcgcgccaagcttgcatgcctgcaggtcgactctagaggatctagaatcaccgaacctcccctcggtacagctcctccagttctaccatgaatttcatccactgattcctcttcaatcgccattgcagattctctcgatctatgctcaaaaaatcccgagataaaaccctagatctgcttcaaatgctctgataccatgtaatttcagtgaattctaactaaacaatggagagaattaactattttagaaagactgattgaaggagaagaagagagaaaaattctatattgaactcatgaaccaaaatgaatgaaaaaaataatgagaagaactatactattacaatctatatatctctatttatattctaatctgaagcagttaatttaactgactctaacaactagactgataggtgtacattttctgttagtgcactgcagtgcatttaactaactgcttaacataaagaatgttgttcgaacttcattcgaatagcttcaatgagaagcaaacatgtgtacctgtaaagacacacagtaaaagtgttaataatgaataaatatgaataaatcaaataataaattaaaaataaaaacacatccaattaacattggaggtcttgaaaatcgatggtaattaacaaagacccttgtgaaatttaagtctgtaattgaaaatttgagtataggttaggggacatttgactattttctcattttctttatctttttcctaatttgtggcagacaagtgaggaggccccactgtaattgattcatgcttttgctttcttgactttttggaacaatactatgcatcatatttggtcttaattattcctctgtttatttccagaattttgagctctatacatctaataacaaagcaagcagaggatatatagtttcatcaactaaaaaggttagtcaactcatctaatatttgctactctcatctctattgaagtacagttatggaaaagtagaagtgatgtaagaaaaatgaaagaactttagtaggttagttggatctaacaaagagaaagggaaataaattgcaggagaaagagagaggttaaatacttactcacaccaccgatttacaacaaatcacttaattgtggttagttaatgtatactttcacctcattaaattattacttacccatgataagttgtattaatttggtattaatatccggtgcgggtgaattcttaccgggtgagagggatggggttggagagtgtggagtgaacagaagcagatgttttagattttttctaagatgacgaaagattcccctcactaatgaaaatatattactatacgctattagagatagaaaggttcggtaccagttggtctcgtttctggatgaaccccatttttacaagtcattttcttcaattcaaatcgcaagtgtacctttatcatcttccactaattaagtcctcttaagttcgcgtgaaaatagtgaaattattgattattcttatcatttcatcttctttctcctgataaagttttatgtactttttatgcatcaggtcttgagaacttggaaaggaaaagtagaatcatggaaaaacgaaaagataatgaagaagcaaacaactcattggtatgttatttgatagagtgaactgtaaagtattgaattgtagatatcatgtggctttaaaaatttgatatgtgttattttggcaggagtcattttctgctcttcgcaaggatgctgccaatgttctggatttcctagagagattaaagaatgaagaagatcaaaaggctgttgatgtggatctgattgaaagcctgaaattgaagctgacatttatttgtacatatgtccagctttcttattccgatttggagaagtttgaagatataatgactagaaaaagacaagaggttgagaatctgcttcaaccaattttggatgatgatggcaaagacgtcgggtgtaaatatgtccttactagcctcgccggtaatatggatgactgtataagcttgtatcatcgttctaaatcagatgccaccatgatggatgagcaattgggcttcctcctcttgaatctctctcatctatccaagcatcgtgctgaaaagatgtttcctggagtgactcaatatgaggttcttcagaatgtatgtggcaacataagagatttccatggattgatagtgaattgttgcattaagcatgagatggttgagaatgtcttatctctgtttcaactgatggctgagagagtaggacgcttcctttgggaggatcaggctgatgaagactctcaactctccgagctagatgaggatgatcagaatgataaagaccctcaactcttcaagctagcacatctactcttgaagattgttccaactgaattggaggttatgcacatatgttataaaactttgaaagcttcaacttcaacagaaattggacgcttcattaagaagctcctggaaacctctccggacattctcagagaatatctgattcatctacaagagcatatgataactgttattacccctaacacttcaggggctcgaaacattcatgtcatgatggaattcctattgattattctttctgatatgccgcccaaggactttattcatcatgacaaactttttgatctcttggctcgtgttgtagcacttaccagggaggtatcaactcttgtacgcgacttggaagagaaattaaggattaaagagagtactgacgaaacaaattgtgcaaccctaaagtttctggaaaatattgaactccttaaggaagatctcaaacatgtttatctgaaagtcccggattcatctcaatattgcttccccatgagtgatggacctctcttcatgcatctgctacagagacacttagatgatttgctggattccaatgcttattcaattgctttgataaaggaacaaattgggctggtgaaagaagacttggaattcataagatcttttttcgcgaatattgagcaaggattgtataaagatctctgggaacgtgttctagatgtggcatatgaggcaaaagatgtcatagattcaattattgttcgagataatggtctcttacatcttattttctcacttcccattaccagaaagaagatgatgcttatcaaagaagaggtctctgatttacatgagaacatttccaagaacagaggtctcatcgttgtgaactctcccaagaaaccagttgagagcaagtcattgacaactgataaaataattgtaggttttggtgaggagacaaacttgatacttagaaagctcaccagtggaccggcagatctagatgtcatttcgatcattggtatgccgggtttaggtaaaactactttggcgtacaaagtatacaatgataaatcagtttctagccatttcgaccttcgtgcatggtgcacggtcgaccaagtatatgacgagaagaagttgttggataaaattttcaatcaagttagtgactcaaattcaaaattgagtgagaatattgatgttgctgataaactacggaaacaattgtttggaaagaggtatcttattgtcttagatgacgtgtgggatactaatacatgggatgagctaacaagaccttttcctgatggtatgaaaggaagtagaattattttgacaactcgagaaaagaaagttgctttgcatggaaagctctacactgatcctcttaaccttcgattgctaagatcagaagaaagttgggagttattagagaaaagggcatttggaaacgagagttgccctgatgaactattggatgttggtaaagaaatagccgaaaattgtaaagggcttcctttggtggtggatctgattgctggaatcattgctgggagggaaaagaaaaagagtgtgtggcttgaagttgtaaataatttgcattcctttattttgaagaatgaagtggaagtgatgaaagttatagaaataagttatgaccacttacctgatcacctgaagccatgcttgctgtactttgcaagtgcgccgaaggactgggtaacgacaatccatgagttgaaacttatttggggttttgaaggatttgtggaaaagacagatatgaagagtctggaagaagtggtgaaaatttatttggatgatttaatttccagtagcttggtaatttgtttcaatgagataggtgattaccctacttgccaacttcatgatcttgtgcatgacttttgtttgataaaagcaagaaaggaaaagttgtgtgatcggataagttcaagtgctccatcagatttgttgccacgtcaaattagcattgattatgatgatgatgaagagcactttgggcttaattttgtcctgttcggttcaaataagaaaaggcattccggtaaacacctctattctttgaccataaatggagatgagctggacgaccatctttctgatacatttcatctaagacacttgaggcttcttagaaccttgcacctggaatcctcttttatcatggttaaagattctttgctgaatgaaatatgcatgttgaatcatttgaggtacttaagcattgggacagaagttaaatctctgcctttgtctttctcaaacctctggaatctagaaatcttgtttgtggataacaaagaatcaaccttgatactattaccgagaatttgggatcttgtaaagttgcaagtgctgttcacgactgcttgttctttctttgatatggatgcagatgaatcaatactgatagcagaggacacaaagttagagaacttgacagcattaggggaactcgtgctttcctattggaaagatacagaggatattttcaaaaggcttcccaatcttcaagtgcttcatttcaaactcaaggagtcatgggattattcaacagagcaatattggttcccgaaattggatttcctaactgaactagaaaaactcactgtagattttgaaagatcaaacacaaatgacagtgggtcctctgcagccataaatcggccatgggattttcactttccttcgagtttgaaaagattgcaattgcatgaatttcctctgacatccgattcactatcaacaatagcgagactgctgaaccttgaagagttgtacctttatcgtacaatcatccatggggaagaatggaacatgggagaagaagacacctttgagaatctcaaatgtttgatgttgagtcaagtgattctttccaagtgggaggttggagaggaatcttttcccacgcttgagaaattagaactgtcggactgtcataatcttgaggagattccgtctagttttggggatatttattccttgaaaattatcgaacttgtaaggagccctcaacttgaaaattccgctctcaagattaaggaatatgctgaagatatgaggggaggggacgagcttcagatccttggccagaaggatatcccgttatttaagtagtttttgagcattatggttgaaaagtagattgcactttgctgggtagattgtatatggttaagaaaattctgttacagttgttatgaaacatttttatttgacttttctgagtttcttttagaaaactcagaagtttttaacaaaaattatagtttttataaatacaatgtggatttgcctttggctgtccaacttggtctgaagtctcatatgctcagagcactatcgttcaacctcaatcaaggtactgatttaaaatgacatctatactactttatcacaaacccaacgaactttcatctcaaaagctaggccaggaagtgaagaggttgtagagagcttataagcactcatgacttccttttctcgaacattcaaccaacgtaggctgaaatcccactctgaacgaaaataagtgtttgtttatcaaattaactctcgtagtagaacactgaaataccttcttctaaacgttcaacaaatgggatttccagcactcaaagtgaatgaaaggttcacattaatcttcaaaaagaattacgacaattcatgaccacaagtacattgacagcaccatttcaacagaagaacaagtcaatgctgcatcttcatcaataatccgagtgtcgaacctccttcctgacactgtcctgtatatgtaaagtttctcaacagggcaactttctggtctcgtatctggatgacccctctcgtctataacttcaacattaagccctggcaacttctggaccaacagcttacatgcttcaaaacttactgaacaattagacatccaaagggatcgcattgtctccagctttgcagcattagccaacagagcctcatcgccaaaggggcagtctctaatctcgaatttgaaaaaattgttgttgtatgactttcctctgacatccgatgcactatcaacaatagcaagactggaggttggagaggaatcctttattatacaatcattcagggagaagaatggaacatgggggaggaagacacttttgagaatctgaaatgtgttagagccacaagctacagaagtattgaatttgtcatgaatatcaacattcttcatcctagttaattctttttcaatttttaatagactctcattttaatcactaatattcttctatttgtgacttcttttctgcaggtggcaactttaaattcataaagtataggattgatgacaaactcgaaaaatatcttaatgaggtgaagtttgagcagtcagcagatggtggttccaactctaagttgacaagcacatactatcccggagggcgatttcaagcctgatgcatatggttagtgtggctagagcagacaggatgtattacctggatatctaccaagacgaatccacaatcagttttatgtcaagcaatacatgaagtaactcccgatagaacagtaaaagcaagatgtgtaggtgtatctcgactctaagagattgtacattcctctttgagatttttactgctaatacaaatttacacctcagaagcgaatctagaatttctagagcatgaatgcaccactaatgaaaggagaaaaaaggaagtatgaagtgggaatttgatccttgtttctaggtatataaaatttatcattcaactatacttcatttagcaaacaactctctttgccattatttctcaaacaagggcttctaatattgctaaactaaagactgtcaaaaggtaagttcatcttcaaactctcttgtttactttatctaaaggggaactatgaaaaacaagaaacatcaggaatgtcccgtaaacaaagcagcctcatgcacaaaacatccaacgttggtaggattaatggagggatcgcatcccaggaggatactgtagaaaaattagtggcttctttcaccgctcaaacccatgatctataggttacatggagacaactttatggttgctcgtaggctcccgtcaattctcataaaccacaacaccaaagttgcatcagacatcatcttcattcacaagctgacaatctccacaagtcttagtcaacttgtaatatgaatattagccaggtagacgtacatatttacaaaattgagtttcctatataatatggtttgaaggaatgaaacatgatggggagggtagataaaataatatatgaggcataaaaataggaaagatatttgtagtgagaggttttgactttttatgctgcttttgatcttcagtttcttgtattctttttctactgctttcctcttctttctcctgagtaaagttttatgtaggtactttttatacgtccgatcgtgagaacttgaaagaaagctctctatagctatgttaggtgcccacataaaaaaatgaaatattacaaaaaccctgataataaaatacactaatctaagatattcactgcaacatacatgcaaaatatatatatataaattttcatgaaaattataacaaataatagatgtgaacatataactttaaaaataatattacatccataaagcttaaattctagatccccggtcgactctagaggatccccactccatccgttcactttgatttgtcatgttgcacttttcgaaagtcaatttgactaatttttaaagctaaattagattacactaattcaatattttaaacagaaaaattagatattcaaaaactatacaaaaaatattatacattgcaattttttgcatatcaatatgataaaaaaatatatcgtaaaatattagtcaaaatttttataatttgactcaaatcatgaaaagtataataattaatagtggacggaggaagtattgtctttccagatttgtggccatttttggtccaagggccattagcagttctcttcattttctacttctgtctcatattagatgggcatcttactaaaaatatttgtctcatattacttgattatttattaaatcaaaaagaattaattaattttttctcattttacccctacaattaatatagttttaaaagttttaaacaaattttgaagaatcaaaatttctttttgcaagagacttattaatataaacaaaggataaaataataaaatttgtcaatttattgacgatcacttaataatcatataaaatagaaaatgtttatctaatatgagacggagaaaatatatcctaaaatatttttggacagatatgtgatattctaaccattcactagactatattatgcattttagccgccaatgacttatttcagctttaattaattaggaaagaggaaactgccaatgaggaagagtaggggcgtagttgctgtcgacgaaaaaaagataatactcactcttttcgatttttatttttatttatcacttttaacctatcatgtaaaaagataattatttttttcatgctttatccttagtattaaacaatttaatagggattattttgtaaaatatttatatgaataattgttttcgtaatgaatttgtccggtcaaacaatgataaataaaaatgaatgaagagagtagaaaacaaaacaaaagaacaagttgacaacttgagagattaaaagggtccaaaacgccttggattttgagattccatatgtgaaatttccatgaaataattgaatttgtattattacaagtcaaactttccatttcattccaactagccatcttggtttcaaaattacacattcattcattcacagatctaatattcttaatagtgatttccacatatggctgaagctttcattcaagttctgctagacaatctcacttctttcctcaaaggggaacttgtattgcttttcggttttcaagatgagttccaaaggctttcaagcatgttttctacaattcaagccgtccttgaagatgctcaggagaagcaactcaacaacaagcctctagaaaattggttgcaaaaactcaatgctgctacatatgaagtcgatgacatcttggatgaatataaaaccaaggccacaagattctcccagtctgaatatggccgttatcatccaaaggttatccctttccgtcacaaggtcgggaaaaggatggaccaagtgatgaaaaaactaaaggcaattgctgaggaaagaaagaattttcatttgcacgaaaaaattgtagagagacaagctgttagacgggaaacaggtactcatcttaaattagtattacaacaactaagtttatattcatttttttggcaattatcaaattcagaaaagggttaaatatactcatgtcctatcgtaaatagtgtatatatacctctcgttgtactttcgatctgaatatacttgtcaaatctggcaagctcagaatcaaattatccaccccaacttttaaatactcgatatctttagaaatccacctgtctaactcatccactacccattccctttgctttgaattcttttctttacctataaacttggaacactcgatccgttttgcttttcttaacaaagcagctcagagaaaagaggttttcttctattctgtttctctgtgtgctgcacttgggtccttaatcccattaaaaacagggcatgttaatcccaacgacggtagcctttcctgacagctgactgtaaattttgtctaacaaagaaaaaaaaagattagacatgtttttccttgtcattgattaggctggatttctttcagagtggaacataggggatatattggaccaaaagtagaatgggtatatatttaaagtatttctgatagaacaggagtatattgtgcgaaaatatcctctattttctgttgtctcctaatgagtttgaatgtaataatattctcatgtggacattgcttgcaccaggttctgtattaaccgaaccgcaggtttatggaagagacaaagagaaagatgagatagtgaaaatcctaataaacaatgttagtgatgcccaacacctttcagtcctcccaatacttggtatggggggattaggaaaaacgactcttgcccaaatggtcttcaatgaccagagagttactgagcatttccattccaaaatatggatttgtgtctcggaagattttgatgagaagaggttaataaaggcaattgtagaatctattgaaggaaggccactacttggtgagatggacttggctccacttcaaaagaagcttcaggagttgctgaatggaaaaagatacttgcttgtcttagatgatgtttggaatgaagatcaacagaagtgggctaatttaagagcagtcttgaaggttggagcaagtggtgcttctgttctaaccactactcgtcttgaaaaggttggatcaattatgggaacattgcaaccatatgaactgtcaaatctgtctcaagaagattgttggttgttgttcatgcaacgtgcatttggacaccaagaagaaataaatccaaaccttgtggcaatcggaaaggagattgtgaaaaaaagtggtggtgtgcctctagcagccaaaactcttggaggtattttgtgcttcaagagagaagaaagagcatgggaacatgtgagagacagtccgatttggaatttgcctcaagatgaaagttctattctgcctgccctgaggcttagttaccatcaacttccacttgatttgaaacaatgctttgcgtattgtgcggtgttcccaaaggatgccaaaatggaaaaagaaaagctaatctctctctggatggcgcatggttttcttttatcaaaaggaaacatggagctagaggatgtgggcgatgaagtatggaaagaattatacttgaggtcttttttccaagagattgaagttaaagatggtaaaacttatttcaagatgcatgatctcatccatgatttggcaacatctctgttttcagcaaacacatcaagcagcaatatccgtgaaataaataaacacagttacacacatatgatgtccattggtttcgccgaagtggtgtttttttacactcttccccccttggaaaagtttatctcgttaagagtgcttaatctaggtgattcgacatttaataagttaccatcttccattggagatctagtacatttaagatacttgaacctgtatggcagtggcatgcgtagtcttccaaagcagttatgcaagcttcaaaatctgcaaactcttgatctacaatattgcaccaagctttgttgtttgccaaaagaaacaagtaaacttggtagtctccgaaatcttttacttgatggtagccagtcattgacttgtatgccaccaaggataggatcattgacatgccttaagactctaggtcaatttgttgttggaaggaagaaaggttatcaacttggtgaactaggaaacctaaatctctatggctcaattaaaatctcgcatcttgagagagtgaagaatgataaggacgcaaaagaagccaatttatctgcaaaagggaatctgcattctttaagcatgagttggaataactttggaccacatatatatgaatcagaagaagttaaagtgcttgaagccctcaaaccacactccaatctgacttctttaaaaatctatggcttcagaggaatccatctcccagagtggatgaatcactcagtattgaaaaatattgtctctattctaattagcaacttcagaaactgctcatgcttaccaccctttggtgatctgccttgtctagaaagtctagagttacactgggggtctgcggatgtggagtatgttgaagaagtggatattgatgttcattctggattccccacaagaataaggtttccatccttgaggaaacttgatatatgggactttggtagtctgaaaggattgctgaaaaaggaaggagaagagcaattccctgtgcttgaagagatgataattcacgagtgcccttttctgaccctttcttctaatcttagggctcttacttccctcagaatttgctataataaagtagctacttcattcccagaagagatgttcaaaaaccttgcaaatctcaaatacttgacaatctctcggtgcaataatctcaaagagctgcctaccagcttggctagtctgaatgctttgaaaagtctaaaaattcaattgtgttgcgcactagagagtctccctgaggaagggctggaaggtttatcttcactcacagagttatttgttgaacactgtaacatgctaaaatgtttaccagagggattgcagcacctaacaaccctcacaagtttaaaaattcggggatgtccacaactgatcaagcggtgtgagaagggaataggagaagactggcacaaaatttctcacattcctaatgtgaatatatatatttaagttatttgctattgtttctttgtttgtgagtctttttggttcctgccattgtgattgcatgtaatttttttctagggttgtttctttatgagtctctctctcattggatgtaattttcttttggaaacaaatctgtcaattgatttgtattatacgctttcagaatctattacttatttgtaattgtttctttgtttgtaaattgtgagtatcttattttatggaattttctgattttattttgaaaacaaatcaatgatttgtaagatccatctgtattatactcccttcgtctcattttatgtgtcacctgtcggatttcgagattcaaacaaatctatctttgatcgtaaatttttaatagatcttttaaacattttgaattatcaattattgtgactttagt

SEQ ID NO: 46 - DNA, 5171 bp, Solanum bulbocastanum
ttgatttgtcatgttgcacttttcgaaagtcaatttgactaatttttaaagctaaattagattacactaattcaatattttaaacagaaaaattagatattcaaaaactatacaaaaaatattatacattgcaattttttgcatatcaatatgataaaaaaatatatcgtaaaatattagtcaaaatttttataatttgactcaaatcatgaaaagtataataattaatagtggacggaggaagtattgtctttccagatttgtggccatttttggtccaagggccattagcagttctcttcattttctacttctgtctcatattagatgggcatcttactaaaaatatttgtctcatattacttgattatttattaaatcaaaaagaattaattaattttttctcattttacccctacaattaatatagttttaaaagttttaaacaaattttgaagaatcaaaatttctttttgcaagagacttattaatataaacaaaggataaaataataaaatttgtcaatttattgacgatcacttaataatcatataaaatagaaaatgtttatctaatatgagacggagaaaatatatcctaaaatatttttggacagatatgtgatattctaaccattcactagactatattatgcattttagccgccaatgacttatttcagctttaattaattaggaaagaggaaactgccaatgaggaagagtaggggcgtagttgctgtcgacgaaaaaaagataatactcactcttttcgatttttatttttatttatcacttttaacctatcatgtaaaaagataattatttttttcatgctttatccttagtattaaacaatttaatagggattattttgtaaaatatttatatgaataattgttttcgtaatgaatttgtccggtcaaacaatgataaataaaaatgaatgaagagagtagaaaacaaaacaaaagaacaagttgacaacttgagagattaaaagggtccaaaacgccttggattttgagattccatatgtgaaatttccatgaaataattgaatttgtattattacaagtcaaactttccatttcattccaactagccatcttggtttcaaaattacacattcattcattcacagatctaatattcttaatagtgatttccacatatggctgaagctttcattcaagttctgctagacaatctcacttctttcctcaaaggggaacttgtattgcttttcggttttcaagatgagttccaaaggctttcaagcatgttttctacaattcaagccgtccttgaagatgctcaggagaagcaactcaacaacaagcctctagaaaattggttgcaaaaactcaatgctgctacatatgaagtcgatgacatcttggatgaatataaaaccaaggccacaagattctcccagtctgaatatggccgttatcatccaaaggttatccctttccgtcacaaggtcgggaaaaggatggaccaagtgatgaaaaaactaaaggcaattgctgaggaaagaaagaattttcatttgcacgaaaaaattgtagagagacaagctgttagacgggaaacaggtactcatcttaaattagtattacaacaactaagtttatattcatttttttggcaattatcaaattcagaaaagggttaaatatactcatgtcctatcgtaaatagtgtatatatacctctcgttgtactttcgatctgaatatacttgtcaaatctggcaagctcagaatcaaattatccaccccaacttttaaatactcgatatctttagaaatccacctgtctaactcatccactacccattccctttgctttgaattcttttctttacctataaacttggaacactcgatccgttttgcttttcttaacaaagcagctcagagaaaagaggttttcttctattctgtttctctgtgtgctgcacttgggtccttaatcccattaaaaacagggcatgttaatcccaacgacggtagcctttcctgacagctgactgtaaattttgtctaacaaagaaaaaaaaagattagacatgtttttccttgtcattgattaggctggatttctttcagagtggaacataggggatatattggaccaaaagtagaatgggtatatatttaaagtatttctgatagaacaggagtatattgtgcgaaaatatcctctattttctgttgtctcctaatgagtttgaatgtaataatattctcatgtggacattgcttgcaccaggttctgtattaaccgaaccgcaggtttatggaagagacaaagagaaagatgagatagtgaaaatcctaataaacaatgttagtgatgcccaacacctttcagtcctcccaatacttggtatggggggattaggaaaaacgactcttgcccaaatggtcttcaatgaccagagagttactgagcatttccattccaaaatatggatttgtgtctcggaagattttgatgagaagaggttaataaaggcaattgtagaatctattgaaggaaggccactacttggtgagatggacttggctccacttcaaaagaagcttcaggagttgctgaatggaaaaagatacttgcttgtcttagatgatgtttggaatgaagatcaacagaagtgggctaatttaagagcagtcttgaaggttggagcaagtggtgcttctgttctaaccactactcgtcttgaaaaggttggatcaattatgggaacattgcaaccatatgaactgtcaaatctgtctcaagaagattgttggttgttgttcatgcaacgtgcatttggacaccaagaagaaataaatccaaaccttgtggcaatcggaaaggagattgtgaaaaaaagtggtggtgtgcctctagcagccaaaactcttggaggtattttgtgcttcaagagagaagaaagagcatgggaacatgtgagagacagtccgatttggaatttgcctcaagatgaaagttctattctgcctgccctgaggcttagttaccatcaacttccacttgatttgaaacaatgctttgcgtattgtgcggtgttcccaaaggatgccaaaatggaaaaagaaaagctaatctctctctggatggcgcatggttttcttttatcaaaaggaaacatggagctagaggatgtgggcgatgaagtatggaaagaattatacttgaggtcttttttccaagagattgaagttaaagatggtaaaacttatttcaagatgcatgatctcatccatgatttggcaacatctctgttttcagcaaacacatcaagcagcaatatccgtgaaataaataaacacagttacacacatatgatgtccattggtttcgccgaagtggtgtttttttacactcttccccccttggaaaagtttatctcgttaagagtgcttaatctaggtgattcgacatttaataagttaccatcttccattggagatctagtacatttaagatacttgaacctgtatggcagtggcatgcgtagtcttccaaagcagttatgcaagcttcaaaatctgcaaactcttgatctacaatattgcaccaagctttgttgtttgccaaaagaaacaagtaaacttggtagtctccgaaatcttttacttgatggtagccagtcattgacttgtatgccaccaaggataggatcattgacatgccttaagactctaggtcaatttgttgttggaaggaagaaaggttatcaacttggtgaactaggaaacctaaatctctatggctcaattaaaatctcgcatcttgagagagtgaagaatgataaggacgcaaaagaagccaatttatctgcaaaagggaatctgcattctttaagcatgagttggaataactttggaccacatatatatgaatcagaagaagttaaagtgcttgaagccctcaaaccacactccaatctgacttctttaaaaatctatggcttcagaggaatccatctcccagagtggatgaatcactcagtattgaaaaatattgtctctattctaattagcaacttcagaaactgctcatgcttaccaccctttggtgatctgccttgtctagaaagtctagagttacactgggggtctgcggatgtggagtatgttgaagaagtggatattgatgttcattctggattccccacaagaataaggtttccatccttgaggaaacttgatatatgggactttggtagtctgaaaggattgctgaaaaaggaaggagaagagcaattccctgtgcttgaagagatgataattcacgagtgcccttttctgaccctttcttctaatcttagggctcttacttccctcagaatttgctataataaagtagctacttcattcccagaagagatgttcaaaaaccttgcaaatctcaaatacttgacaatctctcggtgcaataatctcaaagagctgcctaccagcttggctagtctgaatgctttgaaaagtctaaaaattcaattgtgttgcgcactagagagtctccctgaggaagggctggaaggtttatcttcactcacagagttatttgttgaacactgtaacatgctaaaatgtttaccagagggattgcagcacctaacaaccctcacaagtttaaaaattcggggatgtccacaactgatcaagcggtgtgagaagggaataggagaagactggcacaaaatttctcacattcctaatgtgaatatatatatttaagttatttgctattgtttctttgtttgtgagtctttttggttcctgccattgtgattgcatgtaatttttttctagggttgtttctttatgagtctctctctcattggatgtaattttcttttggaaacaaatctgtcaattgatttgtattatacgctttcagaatctattacttatttgtaattgtttctttgtttgtaaattgtgagtatcttattttatggaattttctgattttattttgaaaacaaatcaatgatttgtaagatccatctgtattatactcccttcgtctcattttatgtgtcacctgtcggatttcgagattcaaacaaatctatctttgatcgtaaatttttaatagatcttttaaacattttgaattatcaattattgtgactttagt

SEQ ID NO: 47 - DNA, 7950 bp, Solanum bulbocastanum
aacctcccctcggtacagctcctccagttctaccatgaatttcatccactgattcctcttcaatcgccattgcagattctctcgatctatgctcaaaaaatcccgagataaaaccctagatctgcttcaaatgctctgataccatgtaatttcagtgaattctaactaaacaatggagagaattaactattttagaaagactgattgaaggagaagaagagagaaaaattctatattgaactcatgaaccaaaatgaatgaaaaaaataatgagaagaactatactattacaatctatatatctctatttatattctaatctgaagcagttaatttaactgactctaacaactagactgataggtgtacattttctgttagtgcactgcagtgcatttaactaactgcttaacataaagaatgttgttcgaacttcattcgaatagcttcaatgagaagcaaacatgtgtacctgtaaagacacacagtaaaagtgttaataatgaataaatatgaataaatcaaataataaattaaaaataaaaacacatccaattaacattggaggtcttgaaaatcgatggtaattaacaaagacccttgtgaaatttaagtctgtaattgaaaatttgagtataggttaggggacatttgactattttctcattttctttatctttttcctaatttgtggcagacaagtgaggaggccccactgtaattgattcatgcttttgctttcttgactttttggaacaatactatgcatcatatttggtcttaattattcctctgtttatttccagaattttgagctctatacatctaataacaaagcaagcagaggatatatagtttcatcaactaaaaaggttagtcaactcatctaatatttgctactctcatctctattgaagtacagttatggaaaagtagaagtgatgtaagaaaaatgaaagaactttagtaggttagttggatctaacaaagagaaagggaaataaattgcaggagaaagagagaggttaaatacttactcacaccaccgatttacaacaaatcacttaattgtggttagttaatgtatactttcacctcattaaattattacttacccatgataagttgtattaatttggtattaatatccggtgcgggtgaattcttaccgggtgagagggatggggttggagagtgtggagtgaacagaagcagatgttttagattttttctaagatgacgaaagattcccctcactaatgaaaatatattactatacgctattagagatagaaaggttcggtaccagttggtctcgtttctggatgaaccccatttttacaagtcattttcttcaattcaaatcgcaagtgtacctttatcatcttccactaattaagtcctcttaagttcgcgtgaaaatagtgaaattattgattattcttatcatttcatcttctttctcctgataaagttttatgtactttttatgcatcaggtcttgagaacttggaaaggaaaagtagaatcatggaaaaacgaaaagataatgaagaagcaaacaactcattggtatgttatttgatagagtgaactgtaaagtattgaattgtagatatcatgtggctttaaaaatttgatatgtgttattttggcaggagtcattttctgctcttcgcaaggatgctgccaatgttctggatttcctagagagattaaagaatgaagaagatcaaaaggctgttgatgtggatctgattgaaagcctgaaattgaagctgacatttatttgtacatatgtccagctttcttattccgatttggagaagtttgaagatataatgactagaaaaagacaagaggttgagaatctgcttcaaccaattttggatgatgatggcaaagacgtcgggtgtaaatatgtccttactagcctcgccggtaatatggatgactgtataagcttgtatcatcgttctaaatcagatgccaccatgatggatgagcaattgggcttcctcctcttgaatctctctcatctatccaagcatcgtgctgaaaagatgtttcctggagtgactcaatatgaggttcttcagaatgtatgtggcaacataagagatttccatggattgatagtgaattgttgcattaagcatgagatggttgagaatgtcttatctctgtttcaactgatggctgagagagtaggacgcttcctttgggaggatcaggctgatgaagactctcaactctccgagctagatgaggatgatcagaatgataaagaccctcaactcttcaagctagcacatctactcttgaagattgttccaactgaattggaggttatgcacatatgttataaaactttgaaagcttcaacttcaacagaaattggacgcttcattaagaagctcctggaaacctctccggacattctcagagaatatctgattcatctacaagagcatatgataactgttattacccctaacacttcaggggctcgaaacattcatgtcatgatggaattcctattgattattctttctgatatgccgcccaaggactttattcatcatgacaaactttttgatctcttggctcgtgttgtagcacttaccagggaggtatcaactcttgtacgcgacttggaagagaaattaaggattaaagagagtactgacgaaacaaattgtgcaaccctaaagtttctggaaaatattgaactccttaaggaagatctcaaacatgtttatctgaaagtcccggattcatctcaatattgcttccccatgagtgatggacctctcttcatgcatctgctacagagacacttagatgatttgctggattccaatgcttattcaattgctttgataaaggaacaaattgggctggtgaaagaagacttggaattcataagatcttttttcgcgaatattgagcaaggattgtataaagatctctgggaacgtgttctagatgtggcatatgaggcaaaagatgtcatagattcaattattgttcgagataatggtctcttacatcttattttctcacttcccattaccagaaagaagatgatgcttatcaaagaagaggtctctgatttacatgagaacatttccaagaacagaggtctcatcgttgtgaactctcccaagaaaccagttgagagcaagtcattgacaactgataaaataattgtaggttttggtgaggagacaaacttgatacttagaaagctcaccagtggaccggcagatctagatgtcatttcgatcattggtatgccgggtttaggtaaaactactttggcgtacaaagtatacaatgataaatcagtttctagccatttcgaccttcgtgcatggtgcacggtcgaccaagtatatgacgagaagaagttgttggataaaattttcaatcaagttagtgactcaaattcaaaattgagtgagaatattgatgttgctgataaactacggaaacaattgtttggaaagaggtatcttattgtcttagatgacgtgtgggatactaatacatgggatgagctaacaagaccttttcctgatggtatgaaaggaagtagaattattttgacaactcgagaaaagaaagttgctttgcatggaaagctctacactgatcctcttaaccttcgattgctaagatcagaagaaagttgggagttattagagaaaagggcatttggaaacgagagttgccctgatgaactattggatgttggtaaagaaatagccgaaaattgtaaagggcttcctttggtggtggatctgattgctggaatcattgctgggagggaaaagaaaaagagtgtgtggcttgaagttgtaaataatttgcattcctttattttgaagaatgaagtggaagtgatgaaagttatagaaataagttatgaccacttacctgatcacctgaagccatgcttgctgtactttgcaagtgcgccgaaggactgggtaacgacaatccatgagttgaaacttatttggggttttgaaggatttgtggaaaagacagatatgaagagtctggaagaagtggtgaaaatttatttggatgatttaatttccagtagcttggtaatttgtttcaatgagataggtgattaccctacttgccaacttcatgatcttgtgcatgacttttgtttgataaaagcaagaaaggaaaagttgtgtgatcggataagttcaagtgctccatcagatttgttgccacgtcaaattagcattgattatgatgatgatgaagagcactttgggcttaattttgtcctgttcggttcaaataagaaaaggcattccggtaaacacctctattctttgaccataaatggagatgagctggacgaccatctttctgatacatttcatctaagacacttgaggcttcttagaaccttgcacctggaatcctcttttatcatggttaaagattctttgctgaatgaaatatgcatgttgaatcatttgaggtacttaagcattgggacagaagttaaatctctgcctttgtctttctcaaacctctggaatctagaaatcttgtttgtggataacaaagaatcaaccttgatactattaccgagaatttgggatcttgtaaagttgcaagtgctgttcacgactgcttgttctttctttgatatggatgcagatgaatcaatactgatagcagaggacacaaagttagagaacttgacagcattaggggaactcgtgctttcctattggaaagatacagaggatattttcaaaaggcttcccaatcttcaagtgcttcatttcaaactcaaggagtcatgggattattcaacagagcaatattggttcccgaaattggatttcctaactgaactagaaaaactcactgtagattttgaaagatcaaacacaaatgacagtgggtcctctgcagccataaatcggccatgggattttcactttccttcgagtttgaaaagattgcaattgcatgaatttcctctgacatccgattcactatcaacaatagcgagactgctgaaccttgaagagttgtacctttatcgtacaatcatccatggggaagaatggaacatgggagaagaagacacctttgagaatctcaaatgtttgatgttgagtcaagtgattctttccaagtgggaggttggagaggaatcttttcccacgcttgagaaattagaactgtcggactgtcataatcttgaggagattccgtctagttttggggatatttattccttgaaaattatcgaacttgtaaggagccctcaacttgaaaattccgctctcaagattaaggaatatgctgaagatatgaggggaggggacgagcttcagatccttggccagaaggatatcccgttatttaagtagtttttgagcattatggttgaaaagtagattgcactttgctgggtagattgtatatggttaagaaaattctgttacagttgttatgaaacatttttatttgacttttctgagtttcttttagaaaactcagaagtttttaacaaaaattatagtttttataaatacaatgtggatttgcctttggctgtccaacttggtctgaagtctcatatgctcagagcactatcgttcaacctcaatcaaggtactgatttaaaatgacatctatactactttatcacaaacccaacgaactttcatctcaaaagctaggccaggaagtgaagaggttgtagagagcttataagcactcatgacttccttttctcgaacattcaaccaacgtaggctgaaatcccactctgaacgaaaataagtgtttgtttatcaaattaactctcgtagtagaacactgaaataccttcttctaaacgttcaacaaatgggatttccagcactcaaagtgaatgaaaggttcacattaatcttcaaaaagaattacgacaattcatgaccacaagtacattgacagcaccatttcaacagaagaacaagtcaatgctgcatcttcatcaataatccgagtgtcgaacctccttcctgacactgtcctgtatatgtaaagtttctcaacagggcaactttctggtctcgtatctggatgacccctctcgtctataacttcaacattaagccctggcaacttctggaccaacagcttacatgcttcaaaacttactgaacaattagacatccaaagggatcgcattgtctccagctttgcagcattagccaacagagcctcatcgccaaaggggcagtctctaatctcgaatttgaaaaaattgttgttgtatgactttcctctgacatccgatgcactatcaacaatagcaagactggaggttggagaggaatcctttattatacaatcattcagggagaagaatggaacatgggggaggaagacacttttgagaatctgaaatgtgttagagccacaagctacagaagtattgaatttgtcatgaatatcaacattcttcatcctagttaattctttttcaatttttaatagactctcattttaatcactaatattcttctatttgtgacttcttttctgcaggtggcaactttaaattcataaagtataggattgatgacaaactcgaaaaatatcttaatgaggtgaagtttgagcagtcagcagatggtggttccaactctaagttgacaagcacatactatcccggagggcgatttcaagcctgatgcatatggttagtgtggctagagcagacaggatgtattacctggatatctaccaagacgaatccacaatcagttttatgtcaagcaatacatgaagtaactcccgatagaacagtaaaagcaagatgtgtaggtgtatctcgactctaagagattgtacattcctctttgagatttttactgctaatacaaatttacacctcagaagcgaatctagaatttctagagcatgaatgcaccactaatgaaaggagaaaaaaggaagtatgaagtgggaatttgatccttgtttctaggtatataaaatttatcattcaactatacttcatttagcaaacaactctctttgccattatttctcaaacaagggcttctaatattgctaaactaaagactgtcaaaaggtaagttcatcttcaaactctcttgtttactttatctaaaggggaactatgaaaaacaagaaacatcaggaatgtcccgtaaacaaagcagcctcatgcacaaaacatccaacgttggtaggattaatggagggatcgcatcccaggaggatactgtagaaaaattagtggcttctttcaccgctcaaacccatgatctataggttacatggagacaactttatggttgctcgtaggctcccgtcaattctcataaaccacaacaccaaagttgcatcagacatcatcttcattcacaagctgacaatctccacaagtcttagtcaacttgtaatatgaatattagccaggtagacgtacatatttacaaaattgagtttcctatataatatggtttgaaggaatgaaacatgatggggagggtagataaaataatatatgaggcataaaaataggaaagatatttgtagtgagaggttttgactttttatgctgcttttgatcttcagtttcttgtattctttttctactgctttcctcttctttctcctgagtaaagttttatgtaggtactttttatacgtccgatcgtgagaacttgaaagaaagctctctatagctatgttaggtgcccacataaaaaaatgaaatattacaaaaaccctgataataaaatacactaatctaagatattcactgcaacatacatgcaaaatatatatatataaattttcatgaaaattataacaaataatagatgtgaacatataactttaaaaataatattacatccataaagcttaaattctaga

SEQ ID NO: 48 - DNA, 2584 bp, Artificial sequence
  synthetic construct sequence
gatcatgagcggagaattaagggagtcacgttatgacccccgccgatgacgcgggacaagccgttttacgtttggaactgacagaaccgcaacgttgaaggagccactcagccgcgggtttctggagtttaatgagctaagcacatacgtcagaaaccattattgcgcgttcaaaagtcgcctaaggtcactatcagctagcaaatatttcttgtcaaaaatgctccactgacgttccataaattcccctcggtatccaattagagtctcatattcactctcaatccagatccccgggtaccatggcggcggcaacaacaacaacaacaacatcttcttcgatctccttctccaccaaaccatctccttcctcctccaaatcaccattaccaatctccagattctccctcccattctccctaaaccccaacaaatcatcctcctcctcccgccgccgcggtatcaaatccagctctccctcctccatctccgccgtgctcaacacaaccaccaatgtcacaaccactccctctccaaccaaacctaccaaacccgaaacattcatctcccgattcgctccagatcaaccccgcaaaggcgctgatattctcgtcgaggctttagaacgtcaaggcgtagaaaccgtattcgcttaccctggaggtgcatcaatggagattcaccaagccttaacccgctcttcctcaatccgtaacgtccttcctcgtcacgaacaaggaggtgtattcgcagcagaaggatacgctcgatcctcaggtaaaccaggtatctgtatagccacttcaggtcccggagctacaaatctcgttagcggattagccgatgcgttgttagatagtgttcctcttgtagcaatcacaggacaagtccctcgtcgtatgattggtacagatgcgtttcaagagactccgattgttgaggtaacgcgttcgattacgaagcataactatcttgtgatggatgttgaagatattcctaggattattgaggaggctttctttttagctacttctggtagacctggacctgttttggttgatgttcctaaagatattcaacaacagcttgcgattcctaattgggaacaggctatgagattacctggttatatgtctaggatgcctaaacctccggaagattctcatttggagcagattgttaggttgatttctgagtctaagaagcctgtgttgtatgttggtggtggttgtttgaactctagcgatgaattgggtaggtttgttgagcttacgggaatccctgttgcgagtacgttgatggggctgggatcttatccttgtgatgatgagttgtcgttacatatgcttggaatgcatgggactgtgtatgcaaattacgctgtggagcatagtgatttgttgttggcgtttggggtaaggtttgatgatcgtgtcacgggtaaacttgaggcttttgctagtagggctaagattgttcatattgatattgactcggctgagattgggaagaataagactcctcatgtgtctgtgtgtggtgatgttaagctggctttgcaagggatgaataaggttcttgagaaccgagcggaggagcttaaacttgattttggagtttggaggaatgagttgaacgtacagaaacagaagtttccgttgagctttaagacgtttggggaagctattcctccacagtatgcgattaaggtccttgatgagttgactgatggaaaagccataataagtactggtgtcgggcaacatcaaatgtgggcggcgcagttctacaattacaagaaaccaaggcagtggctatcatcaggaggccttggagctatgggatttggacttcctgctgcgattggagcgtctgttgctaaccctgatgcgatagttgtggatattgacggagatggaagttttataatgaatgtgcaagagctagccactattcgtgtagagaatcttccagtgaaggtacttttattaaacaaccagcatcttggcatggttatgcaatgggaagatcggttctacaaagctaaccgagcacacacatttctcggagatccggctcaggaggacgagatattcccgaacatgttgctgtttgcagcagcttgcgggattccagcggcgagggtgacaaagaaagcagatctccgagaagctattcagacaatgctggatacaccaggaccttacctgttggatgtgatttgtccgcaccaagaacatgtgttgccgatgatcccgaatggtggcactttcaacgatgtcataacggaaggagatggccggattaaatactgagagctcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatc

SEQ ID NO: 49 - DNA, 27 bp, Artificial sequence
  primer sequence
tgttgaacactgtaacatgctaaaatg

SEQ ID NO: 50 - DNA, 21 bp, Artificial sequence
  primer sequence
agttgtggacatccccgaatt

SEQ ID NO: 51 - DNA, 28 bp, Artificial sequence
  primer sequence
agagggattgcagcacctaacaaccctc

SEQ ID NO: 52 - DNA, 25 bp, Artificial sequence
  primer sequence
ttcaaaaccccaaataagtttcaac

SEQ ID NO: 53 - DNA, 21 bp, Artificial sequence
  primer sequence
ccatgcttgctgtactttgca

SEQ ID NO: 54 - DNA, 20 bp, Artificial sequence
  primer sequence
cgttacccagtccttcggcg

SEQ ID NO: 55 - DNA, 20 bp, Artificial sequence
  primer sequence
aacgatgtcataacggaagg

SEQ ID NO: 56 - DNA, 25 bp, Artificial sequence
  primer sequence
agagcatttgaagcagatctagggt

SEQ ID NO: 57 - DNA, 25 bp, Artificial sequence
  primer sequence
cggattaaatactgagagctcgaat

SEQ ID NO: 58 - DNA, 22 bp, Artificial sequence
  primer sequence
cagatctagggttttatctcgg

SEQ ID NO: 59 - DNA, 20 bp, Artificial sequence
  primer sequence
tgccggtcttgcgatgatta

SEQ ID NO: 60 - DNA, 25 bp, Artificial sequence
  primer sequence
agatctagggttttatctcgggatt

SEQ ID NO: 61 - DNA, 16 bp, Artificial sequence
  primer sequence
tgwgnagwancasaga

SEQ ID NO: 62 - DNA, 16 bp, Artificial sequence
  primer sequence
agwgnagwancawagg

SEQ ID NO: 63 - DNA, 16 bp, Artificial sequence
  primer sequence
cawcgncnganasgaa

SEQ ID NO: 64 - DNA, 16 bp, Artificial sequence
  primer sequence
tcstncgnacntwgga

SEQ ID NO: 65 - DNA, 25 bp, Artificial sequence
  primer sequence
taattcagtacattaaagacgtccg

SEQ ID NO: 66 - DNA, 23 bp, Artificial sequence
  primer sequence
gtcccatagtcatttcttgatca

SEQ ID NO: 67 - DNA, 26 bp, Artificial sequence
  primer sequence
tgtctctgataggctaataaactatg

SEQ ID NO: 68 - DNA, 21 bp, Artificial sequence
  primer sequence
tagatctgattgtcgtttccc

SEQ ID NO: 69 - DNA, 23 bp, Artificial sequence
  primer sequence
atgacgttatttatgagatgggt

SEQ ID NO: 70 - DNA, 20 bp, Artificial sequence
  primer sequence
atttaaaaggcaaaacgtgc

SEQ ID NO: 71 - DNA, 23 bp, Artificial sequence
  primer sequence
ttcatgtcaagttcaatttcagg

SEQ ID NO: 72 - DNA, 21 bp, Artificial sequence
  primer sequence
actcacattaattgcgttgcg

SEQ ID NO: 73 - DNA, 26 bp, Artificial sequence
  primer sequence
gcttggtaataattgtcattagattg

SEQ ID NO: 74 - DNA, 23 bp, artificial
  primer sequence
gccttgacctttgaattatttac

SEQ ID NO: 75 - DNA, 25 bp, Artificial sequence
  primer sequence
tctgatgcagaattttctaactcaa

SEQ ID NO: 76 - DNA, 26 bp, Artificial sequence
  primer sequence
ttcctactagatctgattgtcgtttc